(19) United States
(12) Patent Application Publication
(10) Pub. No.: US 2026/0207505 A1
(43) Pub. Date: Jul. 23, 2026

(54) USE OF GLYCERIDES FOR LNP FORMULATIONS

(71) Applicant: SANOFI PASTEUR INC., Swiftwater, PA (US)

(72) Inventors: Frank DEROSA, Cambridge, MA (US); Shrirang KARVE, Cambridge, MA (US); Nikita KHADSE, Cambridge, MA (US); Ashish SARODE, Cambridge, MA (US)

(21) Appl. No.: 19/414,473

(22) Filed: Dec. 10, 2025

Related U.S. Application Data
(63) Continuation of application No. PCT/IB2024/000334, filed on Jun. 28, 2024.

(30) Foreign Application Priority Data
Jun. 28, 2023 (EP) 23306047.4

(51) Int. Cl. A61K 9/1272; A61K 39/00; A61K 39/02; A61K 39/12; A61P 37/04

(52) U.S. Cl. CPC A61K 9/1272; A61K 39/02; A61K 39/12; A61P 37/04; A61K 2039/55555

(57) ABSTRACT

Provided are lipid nanoparticles for delivering nucleic acids molecules such as mRNA. Also provided are methods of making and using thereof.

### CROSS-REFERENCE TO RELATED APPLICATIONS

This application is a continuation of International Patent Application No. PCT/IB2024/000334, filed Jun. 28, 2024, which claims priority to European Patent Application No. 23306047.4, filed Jun. 28, 2023, the disclosures of which are incorporated herein by reference in their entirety.

### SEQUENCE LISTING

The instant application contains a Sequence Listing which has been submitted electronically in XML format and is hereby incorporated by reference in its entirety. Said XML file, created on Dec. 8, 2025, is named 772050_SA9-383PCCON_ST26.xml and is 4,823 bytes in size.

### BACKGROUND

Effective targeted delivery of biologically active substances such as nucleic acid molecules (e.g., mRNA) represents a continuing medical challenge. In particular, the delivery of nucleic acids to cells is made difficult by their low in vivo stability, propensity toward rapid degradation, and low cell permeability. Thus, there exists a need to develop methods and compositions to facilitate the delivery of therapeutic and/or prophylactics such as nucleic acids to cells.

Lipid-containing nanoparticle compositions have proven effective as transport vehicles into cells and/or intracellular compartments for biologically active substances such as small molecule drugs, proteins, and nucleic acids. Such compositions generally include one or more ionizable (e.g., cationic) lipids, phospholipids including polyunsaturated lipids, cholesterol-based lipids, and/or lipids containing polyethylene glycol (PEGylated lipids). Though a variety of such lipid-containing nanoparticle compositions have been demonstrated, there remains a need for lipid nanoparticle formulations having improved efficacy.

### SUMMARY

The present disclosure provides, inter alia, a composition comprising a lipid nanoparticle (LNP), wherein the LNP comprises: (I) an ionizable lipid; (II) a glyceride or an acylglycol; and (III) one or more lipids selected from the group consisting of: (a) a structural lipid; (b) a helper lipid; and (c) a stealth lipid.

In some embodiments, the LNP comprises: (I) an ionizable lipid; (II) a glyceride or an acylglycol; (III) a structural lipid; (IV) a helper lipid; and (V) a stealth lipid.

In some embodiments, the LNP comprises: (I) an ionizable lipid; (II) a glyceride or an acylglycol having a structure according to Formula I or Formula II, as defined herein; (III) a structural lipid; (IV) a helper lipid; and (V) a stealth lipid.

The present disclosure further provides a composition comprising a lipid nanoparticle (LNP), wherein the LNP comprises: (I) an ionizable lipid having a structure according to Formula CAT-I or CAT-II, as defined herein; (II) a glyceride or an acylglycol; (III) a structural lipid, (IV) a stealth lipid; and (V) a helper lipid.

In some embodiments, the LNP comprises: (I) an ionizable lipid having a structure according to Formula CAT-I or CAT-II, as defined herein; (II) a glyceride or an acylglycol having a structure according to Formula I or II, as defined herein; (III) a structural lipid, (IV) a stealth lipid; and (V) a helper lipid.

The present disclosure further provides an LNP as described herein, further comprising a nucleic acid molecule, wherein the nucleic acid molecule is encapsulated in the LNP. In some embodiments, the nucleic acid molecule is an mRNA molecule.

The present disclosure further provides a method of preventing an infection or reducing one or more symptoms of an infection, comprising administering to the subject, optionally intramuscularly, intranasally, intravenously, subcutaneously, or intradermally, a prophylactically effective amount of a composition described herein.

The present disclosure further provides the use of a composition described herein for the manufacture of a medicament for use in treating a subject in need thereof.

The present disclosure further provides a kit comprising a container comprising a single-use or multi-use dosage of a composition described herein, optionally wherein the container is a vial or a pre-filled syringe or injector.

### DETAILED DESCRIPTION

The present disclosure provides lipid nanoparticle (LNP) formulations for delivering cargo, such as a nucleic acid molecule (e.g., mRNA), to a target cell. In particular, the LNPs of the present disclosure comprise an ionizable lipid, a glyceride or an acylglycol, and at least one of a structural lipid, a helper lipid, and a stealth lipid (e.g., PEGylated). In some embodiments, the LNPs comprise an ionizable lipid, a glyceride or an acylglycol, a structural lipid, a helper lipid, and a stealth lipid.

It has been discovered that addition of a glyceride or an acylglycol to LNP formulations can improve the delivery efficiency of the mRNA, thereby increasing the expression of the protein encoded by the nucleic acid molecule when compared to LNP formulations without a glyceride or an acylglycol. For example, LNP formulations of the present disclosure comprising hEPO mRNA were found to significantly improve protein expression over control formulations.

### Definitions

Unless otherwise defined herein, scientific and technical terms used in this application shall have the meanings that are commonly understood by those of ordinary skill in the art.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps and excludes other ingredients/steps.

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6% 5%, 4%, 3%, 2%, 1%, or less in either direction (greater than or less than) of the stated reference value unless otherwise stated or otherwise evident from the context (except where such number would exceed 100% of a possible value).

As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery).

As used herein, “expression” of a nucleic acid sequence refers to translation of an mRNA into a polypeptide, assemble multiple polypeptides (e.g., heavy chain or light chain of antibody) into an intact protein (e.g., antibody) and/or post-translational modification of a polypeptide or fully assembled protein (e.g., antibody). In this application, the terms “expression” and “production,” and grammatical equivalent, are used inter-changeably.

As used herein, a “functional” biological molecule is a biological molecule in a form in which it exhibits a property and/or activity by which it is characterized.

As used herein, the term “half-life” is the time required for a quantity such as nucleic acid or protein concentration or activity to fall to half of its value as measured at the beginning of a time period.

As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

As used herein, the terms “local distribution,” “local delivery,” or grammatical equivalent, refer to tissue specific delivery or distribution. Typically, local distribution or delivery requires a protein (e.g., enzyme) encoded by mRNAs be translated and expressed intracellularly or with limited secretion that avoids entering the patient's circulation system.

As used herein, the term “messenger RNA (mRNA)” refers to a polynucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, the mRNA comprises one or more nonstandard nucleotide residues. The nonstandard nucleotide residues may include, e.g., 5-methyl-cytidine (“5mC”), pseudouridine (“ψU”), and/or 2-thio-uridine (“2sU”). See, e.g., U.S. Pat. No. 8,278,036 or WO2011012316 for a discussion of such residues and their incorporation into mRNA. The mRNA may be RNA, which is defined as RNA in which 25% of U residues are 2-thio-uridine and 25% of C residues are 5-methylcytidine. Teachings for the use of RNA are disclosed US Patent Publication US20120195936 and international publication WO2011012316, both of which are hereby incorporated by reference in their entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Certain embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In embodiments where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In certain embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA.

The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

As used herein, the terms “systemic distribution,” “systemic delivery,” or grammatical equivalent, refer to a delivery or distribution mechanism or approach that affect the entire body or an entire organism. Typically, systemic distribution or delivery is accomplished via body's circulation system, e.g., blood stream. Compared to the definition of “local distribution or delivery.”

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “target tissues” refers to any tissue that is affected by a disease to be treated. In some embodiments, target tissues include those tissues that display disease-associated pathology, symptom, or feature.

As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

As used herein, the term “treatment” or “treating,” is defined as the application or administration of a therapeutic agent to a patient, or application or administration of a therapeutic agent to an isolated tissue or cell line from a patient (e.g., for diagnosis or ex vivo applications), who has a disorder or disease as described herein, a symptom thereof; or the potential to develop such disorder or disease, where the purpose of the application or administration is to cure, heal, alleviate, relieve, alter, remedy, ameliorate, improve or affect the disorder or disease, or its symptoms. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

Definitions of specific functional groups and chemical terms are described in more detail below.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC) and the formation and crystallization of chiral salts; or preferred isomers can be prepared by asymmetric syntheses. See, for example, Jacques et al., Enantiomers. Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, Ind. 1972). The invention additionally contemplates compounds as individual isomers substantially free of other isomers, and alternatively, as mixtures of various isomers.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄, C₄₋₅, and C₅₋₆ alkyl.

As used herein, “alkyl” refers to a radical of a straight-chain or branched saturated hydrocarbon group having from 1 to 50 carbon atoms (“C1-50 alkyl”). In some embodiments, an alkyl group has 1 to 40 carbon atoms (“C1-40 alkyl”). In some embodiments, an alkyl group has 1 to 30 carbon atoms (“C1-30 alkyl”). In some embodiments, an alkyl group has 1 to 20 carbon atoms (“C1-20 alkyl”). In some embodiments, an alkyl group has 1 to 10 carbon atoms (“C1-10 alkyl”). In some embodiments, an alkyl group has 1 to 9 carbon atoms (“C1-9 alkyl”). In some embodiments, an alkyl group has 1 to 8 carbon atoms (“C1-8 alkyl”). In some embodiments, an alkyl group has 1 to 7 carbon atoms (“C1-7 alkyl”). In some embodiments, an alkyl group has 1 to 6 carbon atoms (“C₁₋₆ alkyl”). In some embodiments, an alkyl group has 1 to 5 carbon atoms (“C1-5 alkyl”). In some embodiments, an alkyl group has 1 to 4 carbon atoms (“C1-4 alkyl”). In some embodiments, an alkyl group has 1 to 3 carbon atoms (“C1-3 alkyl”). In some embodiments, an alkyl group has 1 to 2 carbon atoms (“C1-2 alkyl”). In some embodiments, an alkyl group has 1 carbon atom (“C1 alkyl”). In some embodiments, an alkyl group has 2 to 6 carbon atoms (“C2-6 alkyl”). Examples of C1-6 alkyl groups include, without limitation, methyl (C1), ethyl (C2), n-propyl (C3), isopropyl (C3), n-butyl (C4), tert-butyl (C4), sec-butyl (C4), iso-butyl (C4), n-pentyl (C5), 3-pentanyl (C5), amyl (C5), neopentyl (C5), 3-methyl-2-butanyl (C5), tertiary amyl (C5), and n-hexyl (C6). Additional examples of alkyl groups include n-heptyl (C7), n-octyl (C8) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C1-50 alkyl. In certain embodiments, the alkyl group is a substituted C1-50 alkyl.

As used herein, “heteroalkyl” refers to an alkyl group as defined herein which further includes at least one heteroatom (e.g., 1 to 25, e.g., 1, 2, 3, or 4 heteroatoms) selected from oxygen, sulfur, nitrogen, boron, silicon, and phosphorus within (i.e., inserted between adjacent carbon atoms of) and/or placed at one or more terminal position(s) of the parent chain. In certain embodiments, a heteroalkyl group refers to a saturated group having from 1 to 50 carbon atoms and 1 or more heteroatoms within the parent chain (“heteroC1-50 alkyl”). In certain embodiments, a heteroalkyl group refers to a saturated group having from 1 to 40 carbon atoms and 1 or more heteroatoms within the parent chain (“heteroC1-40 alkyl”). In certain embodiments, a heteroalkyl group refers to a saturated group having from 1 to 30 carbon atoms and 1 or more heteroatoms within the parent chain (“heteroC1-30 alkyl”). In certain embodiments, a heteroalkyl group refers to a saturated group having from 1 to 20 carbon atoms and 1 or more heteroatoms within the parent chain (“heteroC1-20 alkyl”). In certain embodiments, a heteroalkyl group refers to a saturated group having from 1 to 10 carbon atoms and 1 or more heteroatoms within the parent chain (“heteroC1-10 alkyl”). In some embodiments, a heteroalkyl group is a saturated group having 1 to 9 carbon atoms and 1 or more heteroatoms within the parent chain (“heteroC1-9 alkyl”). In some embodiments, a heteroalkyl group is a saturated group having 1 to 8 carbon atoms and 1 or more heteroatoms within the parent chain (“heteroC1-8 alkyl”). In some embodiments, a heteroalkyl group is a saturated group having 1 to 7 carbon atoms and 1 or more heteroatoms within the parent chain (“heteroC1-7 alkyl”). In some embodiments, a heteroalkyl group is a saturated group having 1 to 6 carbon atoms and 1 or more heteroatoms within the parent chain (“heteroC1-6 alkyl”). In some embodiments, a heteroalkyl group is a saturated group having 1 to 5 carbon atoms and 1 or 2 heteroatoms within the parent chain (“heteroC1-5 alkyl”). In some embodiments, a heteroalkyl group is a saturated group having 1 to 4 carbon atoms and 1 or 2 heteroatoms within the parent chain (“heteroC1-4 alkyl”). In some embodiments, a heteroalkyl group is a saturated group having 1 to 3 carbon atoms and 1 heteroatom within the parent chain (“heteroC1-3 alkyl”). In some embodiments, a heteroalkyl group is a saturated group having 1 to 2 carbon atoms and 1 heteroatom within the parent chain (“heteroC1-2 alkyl”). In some embodiments, a heteroalkyl group is a saturated group having 1 carbon atom and 1 heteroatom (“heteroC1 alkyl”). In some embodiments, a heteroalkyl group is a saturated group having 2 to 6 carbon atoms and 1 or 2 heteroatoms within the parent chain (“heteroC2-6 alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC1-50 alkyl. In certain embodiments, the heteroalkyl group is a substituted heteroC1-50 alkyl.

As used herein, “alkenyl” refers to a radical of a straight-chain or branched hydrocarbon group having from 2 to 50 carbon atoms and one or more carbon-carbon double bonds (e.g., 1, 2, 3, or 4 double bonds) (“C2-50 alkenyl”). In some embodiments, an alkenyl group has 2 to 40 carbon atoms (“C2-40 alkenyl”). In some embodiments, an alkenyl group has 2 to 30 carbon atoms (“C2-30 alkenyl”). In some embodiments, an alkenyl group has 2 to 20 carbon atoms (“C2-20 alkenyl”). In some embodiments, an alkenyl group has 2 to 10 carbon atoms (“C2-10 alkenyl”). In some embodiments, an alkenyl group has 2 to 9 carbon atoms (“C2-9 alkenyl”). In some embodiments, an alkenyl group has 2 to 8 carbon atoms (“C2-8 alkenyl”). In some embodiments, an alkenyl group has 2 to 7 carbon atoms (“C2-7 alkenyl”). In some embodiments, an alkenyl group has 2 to 6 carbon atoms (“C2-6 alkenyl”). In some embodiments, an alkenyl group has 2 to 5 carbon atoms (“C2-5 alkenyl”). In some embodiments, an alkenyl group has 2 to 4 carbon atoms (“C2-4 alkenyl”). In some embodiments, an alkenyl group has 2 to 3 carbon atoms (“C2-3 alkenyl”). In some embodiments, an alkenyl group has 2 carbon atoms (“C2 alkenyl”). The one or more carbon-carbon double bonds can be internal (such as in 2-butenyl) or terminal (such as in 1-butenyl). Examples of C2-4 alkenyl groups include, without limitation, ethenyl (C2), 1-propenyl (C3), 2-propenyl (C3), 1-butenyl (C4), 2-butenyl (C4), butadienyl (C4), and the like. Examples of C2-6 alkenyl groups include the aforementioned C2-4 alkenyl groups as well as pentenyl (C5), pentadienyl (C5), hexenyl (C6), and the like. Additional examples of alkenyl include heptenyl (C7), octenyl (C8), octatrienyl (C8), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C2-50 alkenyl. In certain embodiments, the alkenyl group is a substituted C2-50 alkenyl.

As used herein, “heteroalkenyl” refers to an alkenyl group as defined herein which further includes at least one heteroatom (e.g., 1 to 25, e.g., 1, 2, 3, or 4 heteroatoms) selected from oxygen, sulfur, nitrogen, boron, silicon, and phosphorus within (i.e., inserted between adjacent carbon atoms of) and/or placed at one or more terminal position(s) of the parent chain. In certain embodiments, a heteroalkenyl group refers to a group having from 2 to 50 carbon atoms, at least one double bond, and 1 or more heteroatoms within the parent chain (“heteroC2-50 alkenyl”). In certain embodiments, a heteroalkenyl group refers to a group having from 2 to 40 carbon atoms, at least one double bond, and 1 or more heteroatoms within the parent chain (“heteroC2-40 alkenyl”). In certain embodiments, a heteroalkenyl group refers to a group having from 2 to 30 carbon atoms, at least one double bond, and 1 or more heteroatoms within the parent chain (“heteroC2-30 alkenyl”). In certain embodiments, a heteroalkenyl group refers to a group having from 2 to 20 carbon atoms, at least one double bond, and 1 or more heteroatoms within the parent chain (“heteroC2-20 alkenyl”). In certain embodiments, a heteroalkenyl group refers to a group having from 2 to 10 carbon atoms, at least one double bond, and 1 or more heteroatoms within the parent chain (“heteroC2-10 alkenyl”). In some embodiments, a heteroalkenyl group has 2 to 9 carbon atoms at least one double bond, and 1 or more heteroatoms within the parent chain (“heteroC2-9 alkenyl”). In some embodiments, a heteroalkenyl group has 2 to 8 carbon atoms, at least one double bond, and 1 or more heteroatoms within the parent chain (“heteroC2-8 alkenyl”). In some embodiments, a heteroalkenyl group has 2 to 7 carbon atoms, at least one double bond, and 1 or more heteroatoms within the parent chain (“heteroC2-7 alkenyl”). In some embodiments, a heteroalkenyl group has 2 to 6 carbon atoms, at least one double bond, and 1 or more heteroatoms within the parent chain (“heteroC2-6 alkenyl”). In some embodiments, a heteroalkenyl group has 2 to 5 carbon atoms, at least one double bond, and 1 or 2 heteroatoms within the parent chain (“heteroC2-5 alkenyl”). In some embodiments, a heteroalkenyl group has 2 to 4 carbon atoms, at least one double bond, and for 2 heteroatoms within the parent chain (“heteroC2-4 alkenyl”). In some embodiments, a heteroalkenyl group has 2 to 3 carbon atoms, at least one double bond, and 1 heteroatom within the parent chain (“heteroC2-3 alkenyl”). In some embodiments, a heteroalkenyl group has 2 to 6 carbon atoms, at least one double, bond, and 1 or 2 heteroatoms within the parent chain (“heteroC2-6 alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC2-50 alkenyl. In certain embodiments, the heteroalkenyl group is a substituted heteroC2-50 alkenyl.

As used herein, “alkynyl” refers to a radical of a straight-chain or branched hydrocarbon group having from 2 to 50 carbon atoms and one or more carbon-carbon triple bonds (e.g., 1, 2, 3, or 4 triple bonds) and optionally one or more double bonds (e.g., 1, 2, 3, or 4 double bonds) (“C2-50 alkynyl”). An alkynyl group that has one or more triple bonds and one or more double bonds is also referred to as an “ene-yne”. In some embodiments, an alkynyl group has 2 to 40 carbon atoms (“C2-40 alkynyl”). In some embodiments, an alkynyl group has 2 to 30 carbon atoms (“C2-30 alkynyl”). In some embodiments, an alkynyl group has 2 to 20 carbon atoms (“C2-20 alkynyl”). In some embodiments, an alkynyl group has 2 to 10 carbon atoms (“C2-10 alkynyl”). In some embodiments, an alkynyl group has 2 to 9 carbon atoms (“C2-9 alkynyl”). In some embodiments, an alkynyl group has 2 to 8 carbon atoms (“C2-8 alkynyl”). In some embodiments, an alkynyl group has 2 to 7 carbon atoms (“C2-7 alkynyl”). In some embodiments, an alkynyl group has 2 to 6 carbon atoms (“C2-6 alkynyl”). In some embodiments, an alkynyl group has 2 to 5 carbon atoms (“C2-5 alkynyl”). In some embodiments, an alkynyl group has 2 to 4 carbon atoms (“C2-4 alkynyl”). In some embodiments, an alkynyl group has 2 to 3 carbon atoms (“C2-3 alkynyl”). In some embodiments, an alkynyl group has 2 carbon atoms (“C2 alkynyl”). The one or more carbon-carbon triple bonds can be internal (such as in 2-butynyl) or terminal (such as in 1-butynyl). Examples of C2-4 alkynyl groups include, without limitation, ethynyl (C2), 1-propynyl (C3), 2-propynyl (C3), 1-butynyl (C4), 2-butynyl (C4), and the like. Examples of C2-6 alkenyl groups include the aforementioned C2-4 alkynyl groups as well as pentynyl (C5), hexynyl (C6), and the like. Additional examples of alkynyl include heptynyl (C7), octynyl (C8), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C2-50 alkynyl. In certain embodiments, the alkynyl group is a substituted C2-50 alkynyl.

As used herein, “heteroalkynyl” refers to an alkynyl group as defined herein which further includes at least one heteroatom (e.g., 1 to 25, e.g., 1, 2, 3, or 4 heteroatoms) selected from oxygen, sulfur, nitrogen, boron, silicon, and phosphorus within (i.e., inserted between adjacent carbon atoms of) and/or placed at one or more terminal position(s) of the parent chain. In certain embodiments, a heteroalkynyl group refers to a group having from 2 to 50 carbon atoms, at least one triple bond, and 1 or more heteroatoms within the parent chain (“heteroC2-50 alkynyl”). In certain embodiments, a heteroalkynyl group refers to a group having from 2 to 40 carbon atoms, at least one triple bond, and 1 or more heteroatoms within the parent chain (“heteroC2-40 alkynyl”). In certain embodiments, a heteroalkynyl group refers to a group having from 2 to 30 carbon atoms, at least one triple bond, and 1 or more heteroatoms within the parent chain (“heteroC2-30 alkynyl”). In certain embodiments, a heteroalkynyl group refers to a group having from 2 to 20 carbon atoms, at least one triple bond, and 1 or more heteroatoms within the parent chain (“heteroC2-20 alkynyl”). In certain embodiments, a heteroalkynyl group refers to a group having from 2 to 10 carbon atoms, at least one triple bond, and 1 or more heteroatoms within the parent chain (“heteroC2-10 alkynyl”). In some embodiments, a heteroalkynyl group has 2 to 9 carbon atoms, at least one triple bond, and 1 or more heteroatoms within the parent chain (“heteroC2-9 alkynyl”). In some embodiments, a heteroalkynyl group has 2 to 8 carbon atoms, at least one triple bond, and 1 or more heteroatoms within the parent chain (“heteroC2-8 alkynyl”). In some embodiments, a heteroalkynyl group has 2 to 7 carbon atoms, at least one triple bond, and 1 or more heteroatoms within the parent chain (“heteroC2-7 alkynyl”). In some embodiments, a heteroalkynyl group has 2 to 6 carbon atoms, at least one triple bond, and 1 or more heteroatoms within the parent chain (“heteroC2-6 alkynyl”). In some embodiments, a heteroalkynyl group has 2 to 5 carbon atoms, at least one triple bond, and 1 or 2 heteroatoms within the parent chain (“heteroC2-5 alkynyl”). In some embodiments, a heteroalkynyl group has 2 to 4 carbon atoms, at least one triple bond, and for 2 heteroatoms within the parent chain (“heteroC2-4 alkynyl”). In some embodiments, a heteroalkynyl group has 2 to 3 carbon atoms, at least one triple bond, and 1 heteroatom within the parent chain (“heteroC2-3 alkynyl”). In some embodiments, a heteroalkynyl group has 2 to 6 carbon atoms, at least one triple bond, and 1 or 2 heteroatoms within the parent chain (“heteroC2-6 alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC2-50 alkynyl. In certain embodiments, the heteroalkynyl group is a substituted heteroC2-50 alkynyl.

As used herein, “carbocyclyl” or “carbocyclic” refers to a radical of a non-aromatic cyclic hydrocarbon group having from 3 to 10 ring carbon atoms (“C3-10 carbocyclyl”) and zero heteroatoms in the non-aromatic ring system. In some embodiments, a carbocyclyl group has 3 to 8 ring carbon atoms (“C3-8 carbocyclyl”). In some embodiments, a carbocyclyl group has 3 to 7 ring carbon atoms (“C3-7 carbocyclyl”). In some embodiments, a carbocyclyl group has 3 to 6 ring carbon atoms (“C3-6 carbocyclyl”). In some embodiments, a carbocyclyl group has 4 to 6 ring carbon atoms (“C4-6 carbocyclyl”). In some embodiments, a carbocyclyl group has 5 to 6 ring carbon atoms (“C5-6 carbocyclyl”). In some embodiments, a carbocyclyl group has 5 to 10 ring carbon atoms (“C5-10 carbocyclyl”). Exemplary C3-6 carbocyclyl groups include, without limitation, cyclopropyl (C3), cyclopropenyl (C3), cyclobutyl (C4), cyclobutenyl (C4), cyclopentyl (C5), cyclopentenyl (C5), cyclohexyl (C6), cyclohexenyl (C6), cyclohexadienyl (C6), and the like. Exemplary C3-8 carbocyclyl groups include, without limitation, the aforementioned C3-6 carbocyclyl groups as well as cycloheptyl (C7), cycloheptenyl (C7), cycloheptadienyl (C7), cycloheptatrienyl (C7), cyclooctyl (C8), cyclooctenyl (C8), bicyclo[2.2.1]heptanyl (C7), bicyclo[2.2.2]octanyl (C8), and the like. Exemplary C3-10 carbocyclyl groups include, without limitation, the aforementioned C3-8 carbocyclyl groups as well as cyclononyl (C9), cyclononenyl (C9), cyclodecyl (C10), cyclodecenyl (C10), octahydro-1H-indenyl (C9), decahydronaphthalenyl (C10), spiro[4.5]decanyl (C10), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C3-10 carbocyclyl. In certain embodiments, the carbocyclyl group is a substituted C3-10 carbocyclyl.

In some embodiments, “carbocyclyl” or “carbocyclic” is referred to as a “cycloalkyl”, i.e., a monocyclic, saturated carbocyclyl group having from 3 to 10 ring carbon atoms (“C3-10 cycloalkyl”). In some embodiments, a cycloalkyl group has 3 to 8 ring carbon atoms (“C3-8 cycloalkyl”). In some embodiments, a cycloalkyl group has 3 to 6 ring carbon atoms (“C3-6, cycloalkyl”). In some embodiments, a cycloalkyl group has 4 to 6 ring carbon atoms (“C4-6 cycloalkyl”). In some embodiments, a cycloalkyl group has 5 to 6 ring carbon atoms (“C5-6 cycloalkyl”). In some embodiments, a cycloalkyl group has 5 to 10 ring carbon atoms (“C5-10 cycloalkyl”). Examples of C5-6 cycloalkyl groups include cyclopentyl (C5) and cyclohexyl (C5). Examples of C3-6 cycloalkyl groups include the aforementioned C5-6 cycloalkyl groups as well as cyclopropyl (C3) and cyclobutyl (C4). Examples of C3-8 cycloalkyl groups include the aforementioned C3-6 cycloalkyl groups as well as cycloheptyl (C7) and cyclooctyl (C8). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C3-10 cycloalkyl. In certain embodiments, the cycloalkyl group is a substituted C3-10 cycloalkyl.

As used herein, “heterocyclyl” or “heterocyclic” refers to a radical of a 3- to 14-membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-10 membered heterocyclyl”). In some embodiments, a heterocyclyl group is a 5-8 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-8 membered heterocyclyl”). In some embodiments, a heterocyclyl group is a 5-6 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-6 membered heterocyclyl”). In some embodiments, the 5-6 membered heterocyclyl has 1 or more (e.g., 1, 2, or 3) ring heteroatoms selected from oxygen, sulfur, nitrogen, boron, silicon, and phosphorus. In some embodiments, the 5-6 membered heterocyclyl has 1 or 2 ring heteroatoms selected from oxygen, sulfur, nitrogen, boron, silicon, and phosphorus. In some embodiments, the 5-6 membered heterocyclyl has 1 ring heteroatom selected from oxygen, sulfur, nitrogen, boron, silicon, and phosphorus.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pynrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofiiuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C6-14 aryl”). In some embodiments, an aryl group has 6 ring carbon atoms (“C6 aryl”; e.g., phenyl). In some embodiments, an aryl group has 10 ring carbon atoms (“C10 aryl”; e.g., naphthyl such as 1-naphthyl and 2-naphthyl). In some embodiments, an aryl group has 14 ring carbon atoms (“C14 aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C6-14 aryl. In certain embodiments, the aryl group is a substituted C6-14 aryl.

As used herein, “heteroaryl” refers to a radical of a 5-14 membered monocyclic or polycyclic (e.g., bicyclic or tricyclic) 4n+2 aromatic ring system (e.g., having 6, 10, or 14 π electrons shared in a cyclic array) having ring carbon atoms and 1 or more (e.g., 1, 2, 3, or 4 ring heteroatoms) ring heteroatoms provided in the aromatic ring system, wherein each heteroatom is independently selected from oxygen, sulfur, nitrogen, boron, silicon, and phosphorus (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-10 membered heteroaryl”). In some embodiments, a heteroaryl group is a 5-8 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-8 membered heteroaryl”). In some embodiments, a heteroaryl group is a 5-6 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-6 membered heteroaryl”). In some embodiments, the 5-6 membered heteroaryl has 1 or more (e.g., 1, 2, or 3) ring heteroatoms selected from oxygen, sulfur, nitrogen, boron, silicon, and phosphorus. In some embodiments, the 5-6 membered heteroaryl has 1 or 2 ring heteroatoms selected from oxygen, sulfur, nitrogen, boron, silicon, and phosphorus. In some embodiments, the 5-6 membered heteroaryl has 1 ring heteroatom selected from oxygen, sulfur, nitrogen, boron, silicon, and phosphorus. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

As used herein, the term “partially unsaturated” refers to a ring moiety that includes at least one double or triple bond. The term “partially unsaturated” is intended to encompass rings having multiple sites of unsaturation, but is not intended to include aromatic groups (e.g., aryl or heteroaryl moieties) as herein defined.

As used herein, the term “saturated” refers to a ring moiety that does not contain a double or triple bond, i.e., the ring contains all single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

As understood from the above, alkyl, alkenyl, alkynyl, heteroalkyl, heteroalkenyl, heteroalkynyl, carbocyclyl, heterocyclyl, aryl, and heteroaryl groups, as defined herein, are, in certain embodiments, optionally substituted, as defined in the variable definitions for the compounds provided herein. In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

As used herein, the term “halo” or “halogen” refers to fluorine (fluoro, —F), chlorine (chloro, —Cl), bromine (bromo, —Br), or iodine (iodo, —I).

As used herein, a “counterion” is a negatively charged group associated with a positively charged quarternary amine in order to maintain electronic neutrality. Exemplary counterions include halide ions (e.g., F—, Cl—, Br—, I—), NO3-, ClO4-, OH—, H2PO4-, HSO4-, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quaternary nitrogen atoms.

In certain embodiments, the substituent present on a nitrogen atom is a nitrogen protecting group (also referred to as an amino protecting group). Nitrogen protecting groups are well known in the art and include those described in detail in Protecting Groups in Organic Synthesis, T. W. Greene and P. G. M. Wuts, 3rd edition, John Wiley & Sons, 1999, incorporated herein by reference.

In certain embodiments, the substituent present on an oxygen atom is an oxygen protecting group (also referred to as a hydroxyl protecting group). Oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

In certain embodiments, the substituent present on a sulfur atom is a sulfur protecting group (also referred to as a thiol protecting group). Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

As used herein, use of the phrase “at least one instance” refers to one instance, but also encompasses more than one instance, e.g., for example, 1, 2, 3, 4, 5, 6, 7, 8, 9, or 10 instances, and up to 100 instances.

As used herein, a “polymer” refers to a compound comprised of at least 3 (e.g., at least 10, 20, 30, 40, 50, 60, 70, 80, 90, 100, etc.) repeating covalently bound structural units.

“Attached” refers to the covalent attachment of a group.

As used herein, “lipophilic” refers to the ability of a group to dissolve in fats, oils, lipids, and lipophilic non-polar solvents such as hexane or toluene. In general, a lipophilic group refers to an unsubstituted n-alkyl or unsubstituted n-alkenyl group having 6 to 50 carbon atoms, e.g., 6 to 40, 6 to 30, 6 to 20, 8 to 20, 8 to 19, 8 to 18, 8 to 17, 8 to 16, or 8 to 15 carbon atoms.

As used herein, the term “salt” or “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describes pharmaceutically acceptable salts in detail in J. Pharmaceutical Sciences (1977) 66:1-19.

### Compositions of the Present Lipid Nanoparticles

The present disclosure provides a composition comprising a lipid nanoparticle (LNP). The LNP comprises at least an ionizable lipid, a glyceride or an acylglycol, and at least one of a structural lipid, a helper lipid, and a stealth lipid. In some embodiments, the LNP comprises an ionizable lipid, a glyceride or an acylglycol, and a structural lipid. In some embodiments, the LNP comprises an ionizable lipid, a glyceride or an acylglycol, and a helper lipid. In some embodiments, the LNP comprises an ionizable lipid, a glyceride or an acylglycol, and a stealth lipid. In some embodiments, the LNP comprises an ionizable lipid, a glyceride or an acylglycol, a structural lipid, and a helper lipid. In some embodiments, the LNP comprises an ionizable lipid, a glyceride or an acylglycol, a structural lipid, and a stealth lipid. In some embodiments, the LNP comprises an ionizable lipid, a glyceride or an acylglycol, a helper lipid, and a stealth lipid. In some embodiments, the LNP comprises an ionizable lipid, a glyceride or an acylglycol, a structural lipid, a helper lipid, and a stealth lipid.

### Ionizable/Cationic Lipids

An ionizable lipid facilitates mRNA encapsulation and may be a cationic lipid. A cationic lipid affords a positively charged environment at low pH to facilitate efficient encapsulation of the negatively charged mRNA drug substance.

In some embodiments, the ionizable lipid is a cationic lipid.

In some embodiments, the cationic lipid has a structure according to Formula CAT-I:

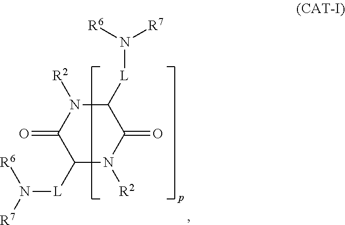

or a pharmaceutically acceptable salt thereof,
wherein:
p is an integer of between 1 and 9, inclusive;
each instance of R² is independently hydrogen or optionally substituted C₁₋₆ alkyl;
each instance of L is independently an optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, or optionally substituted heteroarylene, or combination thereof;
each instance of R⁶ and R⁷ is independently a group of formula (i), (ii), or (iii);

Formulae (i), (ii), and (iii) are:

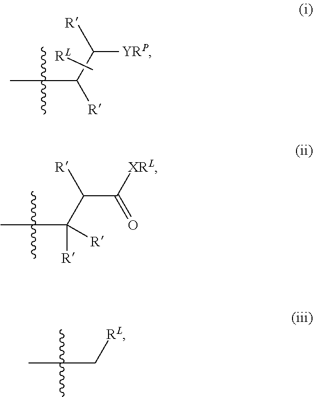

wherein:
each instance of R′ is independently hydrogen or optionally substituted alkyl;
X is O, S, or NR^{X}, wherein R^{X} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
Y is O, S, or NR^{Y}, wherein R^{Y} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
R^{P} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, or a nitrogen protecting group when attached to a nitrogen atom; and
R^{L} is optionally substituted C₁₋₅₀ alkyl, optionally substituted C₂₋₅₀ alkenyl, optionally substituted C₂₋₅₀ alkynyl, optionally substituted heteroC₁₋₅₀ alkyl, optionally substituted heteroC₂₋₅₀ alkenyl, optionally substituted heteroC₂₋₅₀ alkynyl, or a polymer.

In certain embodiments of the lipid of Formula CAT-I, a group of formula (i) represents a group of formula (i-a) or a group of formula (i-b):

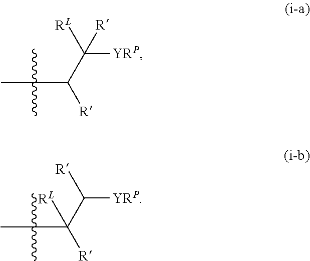

wherein each variable is independently as defined above and described herein. In some embodiments of the lipid of Formula CAT-I, a group of formula (i) is a group of formula (i-a). In some embodiments of the lipid of Formula CAT-I, a group of formula (i) is a group of formula (i-b).

In some embodiments of the lipid of Formula CAT-I, each of R⁶ and R⁷ is independently a group of formula (i). In some embodiments of the lipid of Formula CAT-I, each of R⁶ and R⁷ is independently a group of formula (ii). In some embodiments of the lipid of Formula CAT-I, each of R⁶ and R⁷ is independently a group of formula (iii). In some embodiments of the lipid of Formula CAT-I, each of R⁶ and R⁷ is independently a group of formula (i-a). In some embodiments of the lipid of Formula CAT-I, each of R⁶ and R⁷ is independently a group of formula (i-b).

In some embodiments of the lipid of Formula CAT-I, each instance of R′ is hydrogen.

In some embodiments of the lipid of Formula CAT-I, L is an optionally substituted alkylene.

As generally defined above with respect to the lipid of Formula CAT-I, p is an integer of between 1 and 9, inclusive. In certain embodiments of the lipid of Formula CAT-I, p is 1. In certain embodiments of the lipid of Formula CAT-I, p is 2. In certain embodiments of the lipid of Formula CAT-I, p is 3. In certain embodiments of the lipid of Formula CAT-I, p is 4. In certain embodiments of the lipid of Formula CAT-I, p is 5. In certain embodiments of the lipid of Formula CAT-I, p is 6. In certain embodiments of the lipid of Formula CAT-I, p is 7. In certain embodiments of the lipid of Formula CAT-I, p is 8. In certain embodiments of the lipid of Formula CAT-I, p is 9.

In some embodiments of the lipid of Formula CAT-I, the lipid has a structure according to Formula CAT-Ia:

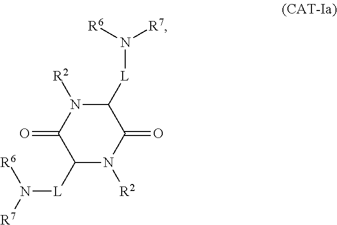

or a pharmaceutically acceptable salt thereof, wherein each variable is independently as defined above and described herein.

In certain embodiments of the lipid of Formula CAT-I, L is an optionally substituted alkylene; e.g., optionally substituted C₁₋₅₀alkylene, optionally substituted C₁₋₄₀alkylene, optionally substituted C₁₋₃₀alkylene, optionally substituted C₁₋₂₀alkylene, optionally substituted C₄₋₂₀alkylene, optionally substituted C₆₋₂₀alkylene, optionally substituted C₈₋₂₀alkylene, optionally substituted C₁₀₋₂₀alkylene, optionally substituted C₁₋₆alkylene, optionally substituted C₂₋₆alkylene, optionally substituted C₃₋₆alkylene, optionally substituted C₄₋₆alkylene, optionally substituted C₄₋₅alkylene, or optionally substituted C₃₋₄alkylene. In some embodiments of the lipid of Formula CAT-I, L is optionally substituted C₁ alkylene. In some embodiments of the lipid of Formula CAT-I, L is optionally substituted C₂ alkylene. In some embodiments of the lipid of Formula CAT-I, L is optionally substituted C₃ alkylene. In some embodiments of the lipid of Formula CAT-I, L is optionally substituted C₄ alkylene. In some embodiments of the lipid of Formula CAT-I, L is optionally substituted C₅ alkylene. In some embodiments of the lipid of Formula CAT-I, L is optionally substituted C₆ alkylene. In some embodiments of the lipid of Formula CAT-I, L is optionally substituted C₇ alkylene. In some embodiments of the lipid of Formula CAT-I, L is optionally substituted C₈ alkylene. In some embodiments of the lipid of Formula CAT-I, L is —CH₂—. In some embodiments of the lipid of Formula CAT-I, L is —(CH₂)₂—. In some embodiments of the lipid of Formula CAT-I, L is —(CH₂)₃—. In some embodiments of the lipid of Formula CAT-I, L is —(CH₂)₄—. In some embodiments of the lipid of Formula CAT-I, L is —(CH₂)₅—. In some embodiments of the lipid of Formula CAT-I, L is —(CH₂)₆—. In some embodiments of the lipid of Formula CAT-I, L is —(CH₂)₇—. In some embodiments of the lipid of Formula CAT-I, L is —(CH₂)₈—.

In certain embodiments of the lipid of Formula CAT-I, L is an optionally substituted alkenylene, e.g., optionally substituted C₂₋₅₀alkenylene, optionally substituted C₂₋₄₀alkenylene, optionally substituted C₂₋₃₀alkenylene, optionally substituted C₂₋₂₀alkenylene, optionally substituted C₄₋₂₀alkenylene, optionally substituted C₆₋₂₀alkenylene, optionally substituted C₈₋₂₀alkenylene, optionally substituted C₁₀₋₂₀alkenylene, optionally substituted C₂₋₆alkenylene, optionally substituted C₃₋₆alkenylene, optionally substituted C₄₋₆alkenylene, optionally substituted C₄₋₅alkenylene, or optionally substituted C₃₋₄alkenylene.

In certain embodiments of the lipid of Formula CAT-I, L is an optionally substituted alkynylene, e.g., optionally substituted C₂₋₅₀alkynylene, optionally substituted C₂₋₄₀alkynylene, optionally substituted C₂₋₃₀alkynylene, optionally substituted C₂₋₂₀alkynylene, optionally substituted C₄₋₂₀alkynylene, optionally substituted C₆₋₂₀alkynylene, optionally substituted C₈₋₂₀alkynylene, optionally substituted C₁₀₋₂₀alkynylene, optionally substituted C₂₋₆alkynylene, optionally substituted C₃₋₆alkynylene, optionally substituted C₄₋₆alkynylene, optionally substituted C₄₋₅alkynylene, or optionally substituted C₃₋₄alkynylene.

In certain embodiments of the lipid of Formula CAT-I, L is an optionally substituted heteroalkylene; e.g., optionally substituted heteroC₁₋₅₀alkylene, optionally substituted heteroC₁₋₄₀alkylene, optionally substituted heteroC₁₋₃₀alkylene, optionally substituted heteroC₁₋₂₀alkylene, optionally substituted heteroC₄₋₂₀alkylene, optionally substituted heteroC₆₋₂₀alkylene, optionally substituted heteroC₈₋₂₀alkylene, optionally substituted heteroC₁₋₂₀alkylene, optionally substituted heteroC₁₋₆alkylene, optionally substituted heteroC₂₋₆alkylene, optionally substituted heteroC₃₋₆-alkylene, optionally substituted heteroC₄₋₆alkylene, optionally substituted heteroC₄₋₅alkylene, or optionally substituted heteroC₃₋₄alkylene.

In certain embodiments of the lipid of Formula CAT-I, L is an optionally substituted heteroalkenylene, e.g., optionally substituted heteroC₂₋₅₀alkenylene, optionally substituted heteroC₂₋₄₀alkenylene, optionally substituted heteroC₂₋₃₀alkenylene, optionally substituted heteroC₂₋₂₀alkenylene, optionally substituted heteroC₄₋₂₀alkenylene, optionally substituted heteroC₆₋₂₀alkenylene, optionally substituted heteroC₈₋₂₀alkenylene, optionally substituted heteroC₁₀₋₂₀alkenylene, optionally substituted heteroC₂₋₆alkenylene, optionally substituted heteroC₃₋₆alkenylene, optionally substituted heteroC₄₋₆alkenylene, optionally substituted heteroC₄₋₅alkenylene, or optionally substituted heteroC₃₋₄alkenylene.

In certain embodiments of the lipid of Formula CAT-I, L is an optionally substituted heteroalkynylene, e.g., optionally substituted heteroC₂₋₅₀alkynylene, optionally substituted heteroC₂₋₄₀alkynylene, optionally substituted heteroC₂₋₃₀alkynylene, optionally substituted heteroC₂₋₂₀alkynylene, optionally substituted heteroC₄₋₂₀alkynylene, optionally substituted heteroC₆₋₂₀alkynylene, optionally substituted heteroC₈₋₂₀alkynylene, optionally substituted heteroC₁₀₋₂₀alkynylene, optionally substituted heteroC₂₋₆alkynylene, optionally substituted heteroC₃₋₆alkynylene, optionally substituted heteroC₄₋₆alkynylene, optionally substituted heteroC₄₋₆alkynylene, or optionally substituted heteroC₃₋₄alkynylene.

In certain embodiments of the lipid of Formula CAT-I, L is an optionally substituted carbocyclylene, e.g., optionally substituted C₃₋₁₀carbocyclylene, optionally substituted C₅₋₈carbocyclylene, optionally substituted C₅₋₆carbocyclylene, optionally substituted C₅carbocyclylene, or optionally substituted C₆carbocyclylene.

In certain embodiments of the lipid of Formula CAT-I, L is an optionally substituted heterocyclylene, e.g., optionally substituted 3-14 membered heterocyclylene, optionally substituted 3-10 membered heterocyclylene, optionally substituted 5-8 membered heterocyclylene, optionally substituted 5-6 membered heterocyclylene, optionally substituted 5-membered heterocyclylene, or optionally substituted 6-membered heterocyclylene.

In certain embodiments of the lipid of Formula CAT-I, L is an optionally substituted arylene, e.g., optionally substituted phenylene. In some embodiments, L is optionally substituted phenylene. In some embodiments, L is substituted phenylene. In some embodiments, L is unsubstituted phenylene.

In certain embodiments of the lipid of Formula CAT-I, L is an optionally substituted heteroarylene, e.g., optionally substituted 5-14 membered heteroarylene, optionally substituted 5-10 membered heteroarylene, optionally substituted 5-6 membered heteroarylene, optionally substituted 5-membered heteroarylene, or optionally substituted 6-membered heteroarylene.

In some embodiments of the lipid of Formula CAT-I, the lipid has a structure according to Formula CAT-Ib:

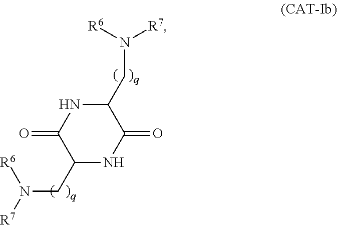

or a pharmaceutically acceptable salt thereof, wherein each variable is independently as defined above and described herein, and wherein 1 is an integer between 1 and 10.

In certain embodiments of the lipid of Formula CAT-Ib, q is an integer between 2 and 10, inclusive. In certain embodiments of the lipid of Formula CAT-Ib, q is an integer between 2 and 8, inclusive. In certain embodiments of the lipid of Formula CAT-Ib, q is an integer between 2 and 6, inclusive. In certain embodiments of the lipid of Formula CAT-Ib, q is 3 or 4. In certain embodiments of the lipid of Formula CAT-Ib, q is 1. In certain embodiments of the lipid of Formula CAT-Ib, q is 2. In certain embodiments of the lipid of Formula CAT-Ib, q is 3. In certain embodiments of the lipid of Formula CAT-Ib, q is 4. In certain embodiments of the lipid of Formula CAT-Ib, q is 5. In certain embodiments of the lipid of Formula CAT-Ib, q is 6. In certain embodiments of the lipid of Formula CAT-Ib, q is 7. In certain embodiments of the lipid of Formula CAT-Ib, q is 8.

In some embodiments of the lipid of Formula CAT-I, R⁶ is a group of formula (i). In some embodiments of the lipid of Formula CAT-I, R⁶ is a group of formula (i-a). In some embodiments of the lipid of Formula CAT-I, R⁶ is a group of formula (i-a1):

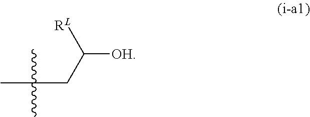

In some embodiments of the lipid of Formula CAT-I, R⁶ is a group of formula (i-b). In some embodiments of the lipid of Formula CAT-I, R⁶ is a group of formula (ii). In some embodiments of the lipid of Formula CAT-I, R⁶ is a group of formula (iii).

In some embodiments of the lipid of Formula CAT-I, R⁷ is a group of formula (i). In some embodiments of the lipid of Formula CAT-I, R⁷ is a group of formula (i-a). In some embodiments of the lipid of Formula CAT-I, R⁷ is a group of formula (i-a1). In some embodiments of the lipid of Formula CAT-I, R⁷ is a group of formula (i-b). In some embodiments of the lipid of Formula CAT-I, R⁷ is a group of formula (ii). In some embodiments of the lipid of Formula CAT-I, R⁷ is a group of formula (iii).

In some embodiments of the lipid of Formula CAT-I, each instance of R⁶ and R⁷ is independently a group of the formula (i). In some embodiments of the lipid of Formula CAT-I, each instance of R⁶ and R⁷ is independently a group of the formula (i-a). In some embodiments of the lipid of Formula CAT-I, each instance of R⁶ and R⁷ is independently a group of the formula (i-b). In some embodiments of the lipid of Formula CAT-I, each instance of R⁶ and R⁷ is independently a group of the formula (ii). In some embodiments of the lipid of Formula CAT-I, each instance of R⁶ and R⁷ is independently a group of the formula (iii).

In some embodiments of the lipid of Formula CAT-I, R⁶ and R⁷ are the same. In some embodiments of the lipid of Formula CAT-I, R⁶ and R⁷ are different.

In some embodiments of the lipid of Formula CAT-I, R⁶ and R⁷ are the same group of formula (i-a1):

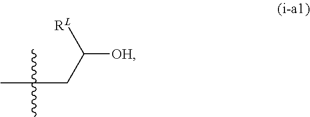

wherein R^{L} is as defined above and described herein.

In some embodiments of the lipid of Formula CAT-I, R⁶ and R⁷ are the same group of formula

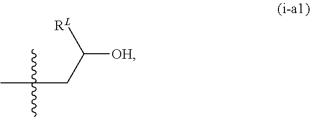

wherein R^{L} is optionally substituted C₁₋₅₀alkyl, optionally substituted C₂₋₅₀alkenyl, optionally substituted C₂₋₅₀alkynyl, optionally substituted heteroC₁₋₅₀alkyl, optionally substituted heteroC₂₋₅₀alkenyl, or optionally substituted heteroC₂₋₅₀alkynyl.

In some embodiments of the lipid of Formula CAT-I, R⁶ and R⁷ are the same group of formula

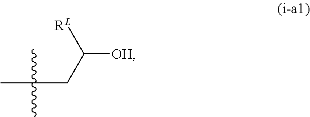

wherein R is optionally substituted C₅₋₂₅alkyl, optionally substituted C₅₋₂₅alkenyl, optionally substituted C₅₋₂₅alkynyl, optionally substituted heteroC₂₋₂₅alkyl, optionally substituted heteroC₅₋₂₅ alkenyl, or optionally substituted heteroC₅₋₂₅alkynyl.

In some embodiments of the lipid of Formula CAT-I, R⁶ and R⁷ are the same group of formula

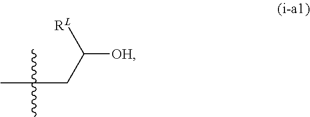

wherein R^{L} is optionally substituted C₅₋₁₅alkyl, optionally substituted C₅₋₁₅alkenyl, optionally substituted C₅₋₁₅alkynyl, optionally substituted heteroC₅₋₁₅alkyl, optionally substituted heteroC₅₋₁₅alkenyl, or optionally substituted heteroC₅₋₁₅alkynyl.

In some embodiments of the lipid of Formula CAT-I, R⁶ and R⁷ are the same group of formula

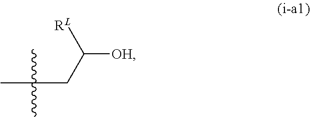

wherein R^{L} is optionally substituted C₁₋₅₀alkyl.

In some embodiments of the lipid of Formula CAT-I, R⁶ and R⁷ are the same group of formula

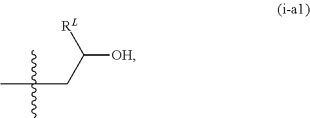

wherein R^{L} is optionally substituted C₅₋₂₅alkyl.

In some embodiments of the lipid of Formula CAT-I, R⁶ and R⁷ are the same group of formula

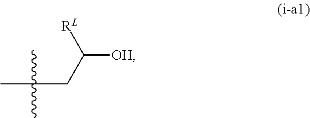

wherein R^{L} is optionally substituted C₅₋₂₀alkyl.

In some embodiments of the lipid of Formula CAT-I, R⁶ and R⁷ are the same group of formula

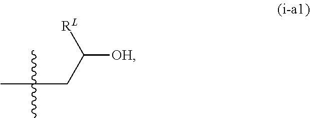

wherein R^{L} is optionally substituted C₅₋₁₅alkyl.

In some embodiments of the lipid of Formula CAT-I, R² is hydrogen. In some embodiments of the lipid of Formula CAT-I, at least one instance of R² is hydrogen. In some embodiments of the lipid of Formula CAT-I, each instance of R² is hydrogen.

In certain embodiments of the lipid of Formula CAT-I, R² is optionally substituted C₁₋₆ alkyl, optionally substituted C₂₋₆alkyl, optionally substituted C₃₋₆alkyl, optionally substituted C₄₋₆alkyl, optionally substituted C₄₋₅alkyl, or optionally substituted C₃₋₄alkyl. In certain embodiments of the lipid of Formula CAT-I, at least one instance of R² is optionally substituted C₁₋₆alkyl.

As generally defined above with respect to the lipid of Formula CAT-I, each instance of R′ is independently hydrogen or optionally substituted alkyl. In some embodiments of the lipid of Formula CAT-I, R′ is hydrogen. In some embodiments of the lipid of Formula CAT-I, R′ is substituted alkyl. In certain embodiments of the lipid of Formula CAT-I, at least one instance of R′ is hydrogen. In certain embodiments of the lipid of Formula CAT-I, at least two instances of R′ are hydrogen. In certain embodiments of the lipid of Formula CAT-I, each instance of R′ is hydrogen. In certain embodiments of the lipid of Formula CAT-I, at least one instance of R′ is optionally substituted alkyl, e.g., methyl. In certain embodiments of the lipid of Formula CAT-I, at least two instances of R′ are optionally substituted alkyl, e.g., methyl. In some embodiments of the lipid of Formula CAT-I, at least one instance of R′ is hydrogen, and at least one instance of R′ is optionally substituted alkyl. In certain embodiments of the lipid of Formula CAT-I, one instance of R′ is optionally substituted alkyl, and the rest are hydrogen.

As generally defined above with respect to the lipid of Formula CAT-I, X is O, S, or NR^{X}. In some embodiments of the lipid of Formula CAT-I, X is O. In some embodiments of the lipid of Formula CAT-I, X is S. In some embodiments of the lipid of Formula CAT-I, X is NRN, wherein R^{X} is as defined above and described herein.

As generally defined above with respect to the lipid of Formula CAT-I, R^{X} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In some embodiments of the lipid of Formula CAT-I, R^{X} is hydrogen. In some embodiments of the lipid of Formula CAT-I, R^{X} is optionally substituted alkyl. In some embodiments of the lipid of Formula CAT-I, R^{Y} is optionally substituted alkenyl. In some embodiments of the lipid of Formula CAT-I, R^{X} is optionally substituted alkynyl. In some embodiments of the lipid of Formula CAT-I, R^{X} is optionally substituted carbocyclyl. In some embodiments of the lipid of Formula CAT-I, R^{X} is optionally substituted heterocyclyl. In some embodiments of the lipid of Formula CAT-I, R^{X} is optionally substituted aryl. In some embodiments of the lipid of Formula CAT-I, R^{X} is optionally substituted heteroaryl. In some embodiments of the lipid of Formula CAT-I, RN is a nitrogen protecting group.

As generally defined above with respect to the lipid of Formula CAT-I, Y is O, S, or NR^{Y}. In some embodiments of the lipid of Formula CAT-I, Y is O. In some embodiments of the lipid of Formula CAT-I, Y is S. In some embodiments of the lipid of Formula CAT-I, Y is NR^{Y}, wherein R is as defined above and described herein.

As generally defined above with respect to the lipid of Formula CAT-I, R^{Y} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In some embodiments of the lipid of Formula CAT-I, R^{Y} is hydrogen. In some embodiments of the lipid of Formula CAT-I, R^{Y} is optionally substituted alkyl. In some embodiments of the lipid of Formula CAT-I, R^{Y} is optionally substituted alkenyl. In some embodiments of the lipid of Formula CAT-I, R^{Y} is optionally substituted alkynyl. In some embodiments of the lipid of Formula CAT-I, R^{Y} is optionally substituted carbocyclyl. In some embodiments of the lipid of Formula CAT-I, R^{Y} is optionally substituted heterocyclyl. In some embodiments of the lipid of Formula CAT-I, R^{Y} is optionally substituted aryl. In some embodiments of the lipid of Formula CAT-I, R is optionally substituted heteroaryl. In some embodiments of the lipid of Formula CAT-I, R^{Y} is a nitrogen protecting group.

As generally defined above with respect to the lipid of Formula CAT-I, R^{P} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, or a nitrogen protecting group when attached to a nitrogen atom. In some embodiments of the lipid of Formula CAT-I, R^{P} is hydrogen. In some embodiments of the lipid of Formula CAT-I, R^{P} is optionally substituted alkyl. In some embodiments of the lipid of Formula CAT-I, R^{P} is optionally substituted alkenyl. In some embodiments of the lipid of Formula CAT-I, R^{P} is optionally substituted alkynyl. In some embodiments of the lipid of Formula CAT-I, R^{P} is optionally substituted carbocyclyl. In some embodiments of the lipid of Formula CAT-I, R^{P} is optionally substituted heterocyclyl. In some embodiments of the lipid of Formula CAT-I, R^{P} is optionally substituted aryl. In some embodiments of the lipid of Formula CAT-I, R^{P} is optionally substituted heteroaryl. In some embodiments of the lipid of Formula CAT-I, R^{P} is an oxygen protecting group when attached to an oxygen atom. In some embodiments of the lipid of Formula CAT-I, R^{P} is a sulfur protecting group when attached to a sulfur atom. In some embodiments of the lipid of Formula CAT-I, R^{P} is a nitrogen protecting group when attached to a nitrogen atom.

As generally defined above with respect to the lipid of Formula CAT-I, R^{L} is optionally substituted C₁₋₅₀ alkyl, optionally substituted C₂₋₅₀ alkenyl, optionally substituted C₂₋₅₀ alkynyl, optionally substituted heteroC₁₋₅₀alkyl, optionally substituted heteroC₂₋₅₀alkenyl, optionally substituted heteroC₂₋₅₀ alkynyl, or a polymer.

In some embodiments of the lipid of Formula CAT-I, R^{L} is optionally substituted C₁₋₅₀ alkyl. In some embodiments of the lipid of Formula CAT-I, R^{L} is optionally substituted C₂₋₃₀ alkyl. In some embodiments of the lipid of Formula CAT-I, R^{L} is optionally substituted C₂₋₂₀ alkyl. In some embodiments of the lipid of Formula CAT-I, R^{L} is optionally substituted C₂₋₁₅ alkyl. In some embodiments of the lipid of Formula CAT-I, R^{L} is optionally substituted C₂₋₁₀ alkyl.

In some embodiments of the lipid of Formula CAT-I, R^{L} is optionally substituted C₆₋₅₀ alkyl. In some embodiments of the lipid of Formula CAT-I, R^{L} is optionally substituted C₆₋₃₀ alkyl. In some embodiments of the lipid of Formula CAT-I, R^{L} is optionally substituted C₆₋₂₀ alkyl. In some embodiments of the lipid of Formula CAT-I, R^{L} is optionally substituted C₆₋₁₅ alkyl. In some embodiments of the lipid of Formula CAT-I, R^{L} is optionally substituted C₆₋₁₀ alkyl.

In some embodiments of the lipid of Formula CAT-I, for example, in any of the above embodiments, R^{L} is a substituted alkyl group. In some embodiments of the lipid of Formula CAT-I, R^{L} Is an unsubstituted alkyl group. In some embodiments of the lipid of Formula CAT-I, R^{L} is an optionally substituted straight-chain alkyl group. In some embodiments of the lipid of Formula CAT-I, R^{L} is a substituted straight-chain alkyl group. In some embodiments of the lipid of Formula CAT-I, R^{L} is an unsubstituted straight-chain alkyl group. In some embodiments of the lipid of Formula CAT-I, R^{L} is an optionally substituted branched alkyl group. In some embodiments of the lipid of Formula CAT-I, R^{L} is a substituted branched alkyl group. In some embodiments of the lipid of Formula CAT-I, R^{L} is an unsubstituted branched alkyl group.

In certain embodiments of the lipid of Formula CAT-I, at least one instance of R^{L} is an unsubstituted alkyl. Exemplary unsubstituted alkyl groups include, but are not limited to, —CH₃, —C₂H₅, —C₃H₇, —C₄H₉, —C₅H₁₁, —C₆H₁₁, —C₇H₁₅, —C₈H₁₇, —C₉H₁₉, —C₁₀H₂₁, —C₁₁H₂₃, —C₁₂H₂₅, —C₁₃H₂₇, —C₁₄H₂₉, —C₁₅H₃₁, —C₁₆H₃₃, —C₁₇H₃₅, —C₁₈H₃₇, —C₁₉H₃₉, —C₂₀H₄₁—, C₂₁H₄₃, —C₂₂H₄₅, —C₂₃H₄₇, —C₂₄H₄₉, and —C₂₅H₅₁.

In certain embodiments of the lipid of Formula CAT-I, at least one instance of R^{L} is a substituted alkyl. For example, in certain embodiments of the lipid of Formula CAT-I, at least one instance of R^{L} is an alkyl substituted with one or more fluorine substituents. Exemplary fluorinated alkyl groups include, but are not limited to:

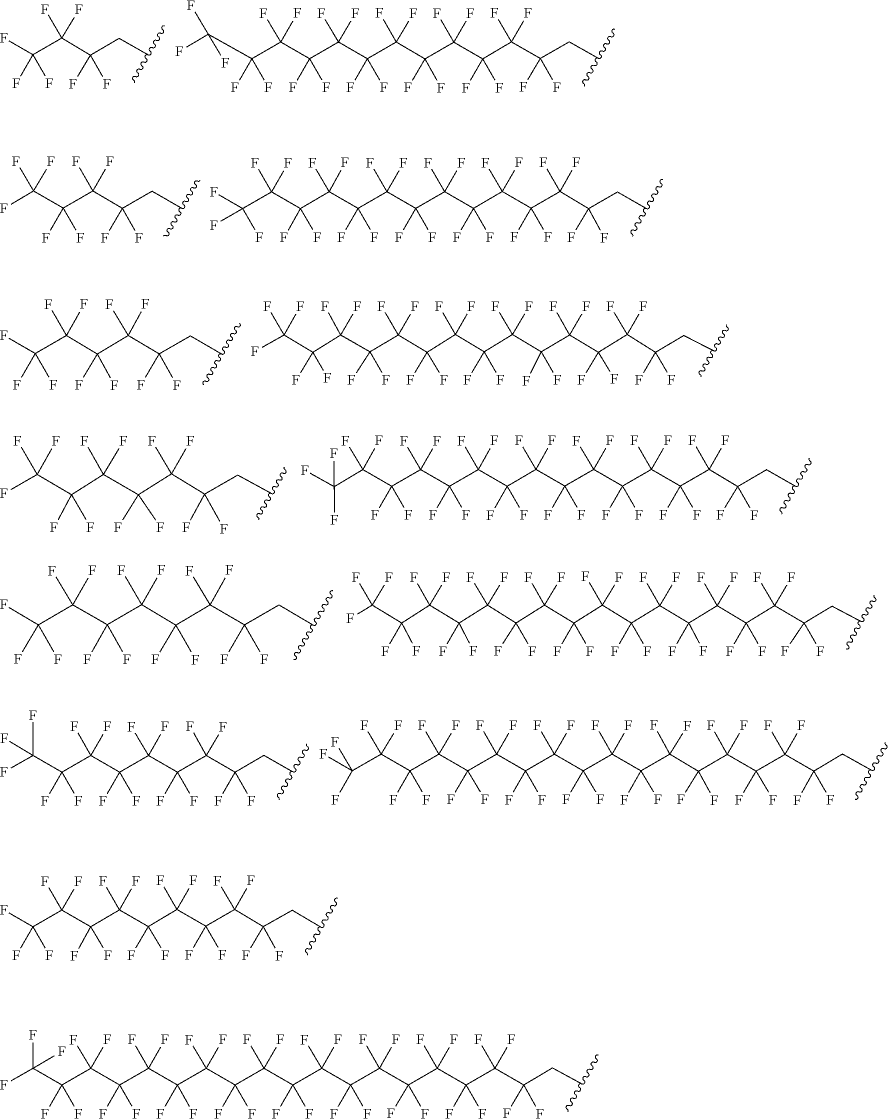

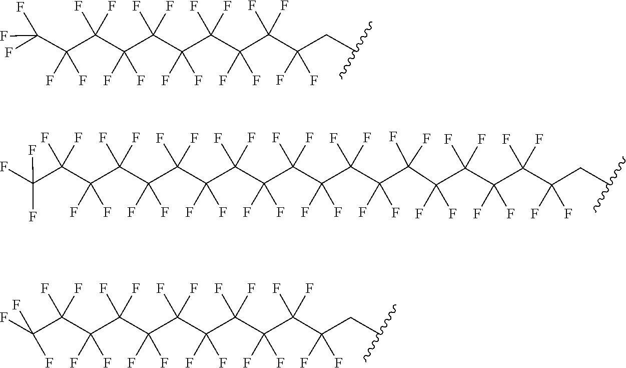

In some embodiments of the lipid of Formula CAT-I, R^{L} is optionally substituted C₂₋₅₀ alkenyl. In some embodiments of the lipid of Formula CAT-I, R^{L} is optionally substituted C₂₋₃₀ alkenyl. In some embodiments of the lipid of Formula CAT-I, R^{L} is optionally substituted C₂₋₂₀ alkenyl. In some embodiments of the lipid of Formula CAT-I, R^{L} is optionally substituted C₂₋₁₈ alkenyl. In some embodiments of the lipid of Formula CAT-I, R^{L} is optionally substituted C₂₋₁₅ alkenyl. In some embodiments of the lipid of Formula CAT-I, R^{L} is optionally substituted C₂₋₁₀ alkenyl.

In some embodiments of the lipid of Formula CAT-I, R^{L} is optionally substituted C₆₋₅₀ alkenyl. In some embodiments of the lipid of Formula CAT-I, R^{L} is optionally substituted C₆₋₃₀ alkenyl. In some embodiments of the lipid of Formula CAT-I, R^{L} is optionally substituted C₆₋₂₀ alkenyl. In some embodiments of the lipid of Formula CAT-I, R^{L} is optionally substituted C₆₋₁₈ alkenyl. In some embodiments of the lipid of Formula CAT-I, R^{L} is optionally substituted C₆₋₁₅ alkenyl. In some embodiments of the lipid of Formula CAT-I, R^{L} is optionally substituted C₆₋₁₀ alkenyl.

In some embodiments of the lipid of Formula CAT-I, for example, in any of the above embodiments, R^{L} is a substituted alkyl group. In some embodiments of the lipid of Formula CAT-I, R^{L} is an unsubstituted alkyl group. In some embodiments of the lipid of Formula CAT-I, R^{L} is an optionally substituted straight-chain alkenyl group. In some embodiments of the lipid of Formula CAT-I, R^{L} is a substituted straight-chain alkenyl group. In some embodiments of the lipid of Formula CAT-I, R^{L} is an unsubstituted straight-chain alkenyl group. In some embodiments of the lipid of Formula CAT-I, R^{L} is an optionally substituted branched alkenyl group. In some embodiments of the lipid of Formula CAT-I, R^{L} is a substituted branched alkenyl group. In some embodiments of the lipid of Formula CAT-I, R^{L} is an unsubstituted branched alkenyl group.

Exemplary unsubstituted alkenyl group include, but are not limited to:

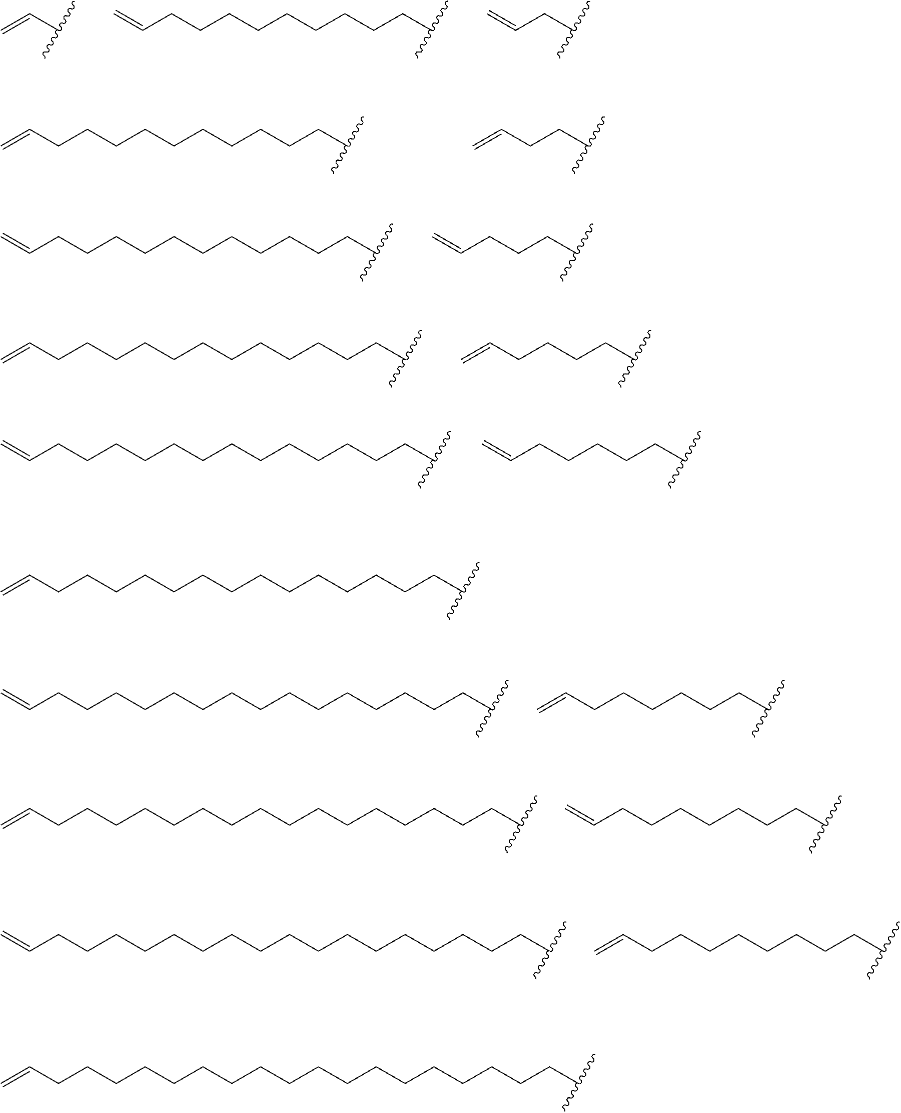

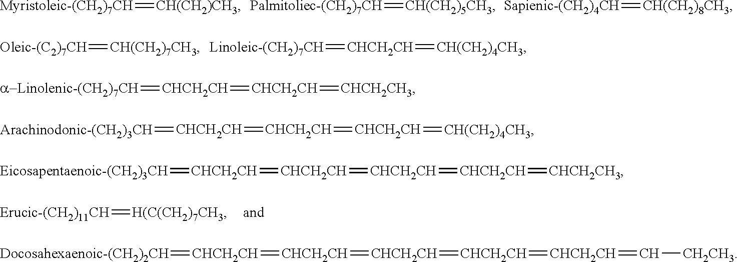

In some embodiments of the lipid of Formula CAT-I, wherein R^{L} is defined as a C₆₋₅₀alkyl or C₆₋₅₀alkenyl groups, such groups are meant to encompass lipophilic groups (also referred to as a “lipid tail”). Lipophilic groups comprise a group of molecules that include fats, waxes, oils, fatty acids, and the like. Lipid tails present in these lipid groups can be saturated and unsaturated, depending on whether or not the lipid tail comprises double bonds. The lipid tail can also comprise different lengths, often categorized as medium (i.e., with tails between 7-12 carbons, e.g., C₇₋₁₂ alkyl or C₇₋₁₂ alkenyl), long (i.e., with tails greater than 12 carbons and up to 22 carbons, e.g., C₁₃₋₂₂alkyl or C₁₃₋₂₂ alkenyl), or very long (i.e., with tails greater than 22 carbons, e.g., C₂₃₋₃₀ alkyl or C₂₃₃₀ alkenyl).

In some embodiments of the lipid of Formula CAT-I, R^{L} is optionally substituted C₂₋₅₀ alkynyl. In some embodiments of the lipid of Formula CAT-I, R^{L} is optionally substituted C₂₋₃₀ alkynyl. In some embodiments of the lipid of Formula CAT-I, R^{L} is optionally substituted C₂₋₂₀ alkynyl. In some embodiments of the lipid of Formula CAT-I, R^{L} is optionally substituted C₂₋₁₅ alkynyl. In some embodiments of the lipid of Formula CAT-I, R^{L} is optionally substituted C₂₋₁₀ alkynyl.

In some embodiments of the lipid of Formula CAT-I, R^{L} is optionally substituted C₆₋₅₀ alkynyl. In some embodiments of the lipid of Formula CAT-I, R^{L} is optionally substituted C₆₋₃₀ alkynyl. In some embodiments of the lipid of Formula CAT-I, R^{L} is optionally substituted C₆₋₂₀ alkynyl. In some embodiments of the lipid of Formula CAT-I, R^{L} is optionally substituted C₆₋₁₅ alkynyl. In some embodiments of the lipid of Formula CAT-I, R^{L} is optionally substituted C₆₋₁₀ alkynyl.

In some embodiments of the lipid of Formula CAT-I, for example, in any of the above embodiments, R^{L} is a substituted alkynyl group. In some embodiments of the lipid of Formula CAT-I, R^{L} is an unsubstituted alkynyl group. In some embodiments of the lipid of Formula CAT-I, R^{L} is an optionally substituted straight-chain alkynyl group. In some embodiments of the lipid of Formula CAT-I, R^{L} is an optionally substituted straight-chain alkynyl group. In some embodiments of the lipid of Formula CAT-I, R^{L} is a substituted straight-chain alkynyl group. In some embodiments of the lipid of Formula CAT-I, R^{L} is an unsubstituted straight-chain alkynyl group. In some embodiments of the lipid of Formula CAT-I, R^{L} is an optionally substituted branched alkynyl group. In some embodiments of the lipid of Formula CAT-I, R^{L} is a substituted branched alkynyl group. In some embodiments of the lipid of Formula CAT-I, R^{L} is an unsubstituted branched alkynyl group.

In some embodiments of the lipid of Formula CAT-I, R^{L} is optionally substituted heteroC₁₋₅₀alkyl. In some embodiments of the lipid of Formula CAT-I, R^{L} is optionally substituted heteroC₂₋₃₀alkyl. In some embodiments of the lipid of Formula CAT-I, R^{L} is optionally substituted heteroC₂₋₂₀alkyl. In some embodiments of the lipid of Formula CAT-I, R^{L} is optionally substituted heteroC₂₋₁₅alkyl. In some embodiments of the lipid of Formula CAT-I, R^{L} is optionally substituted heteroC₂₋₁₀alkyl.

In some embodiments of the lipid of Formula CAT-I, R^{L} is optionally substituted heteroC₆₋₅₀alkyl. In some embodiments of the lipid of Formula CAT-I, R^{L} is optionally substituted heteroC₆₋₃₀alkyl. In some embodiments of the lipid of Formula CAT-I, R^{L} is optionally substituted heteroC₆₋₂₀alkyl. In some embodiments of the lipid of Formula CAT-I, R^{L} is optionally substituted heteroC₆₋₁₅alkyl. In some embodiments of the lipid of Formula CAT-I, R^{L} is optionally substituted heteroC₆₋₁₀alkyl.

In some embodiments of the lipid of Formula CAT-I, for example, in any of the above embodiments, R^{L} is a substituted heteroalkyl group. In some embodiments of the lipid of Formula CAT-I, R^{L} is an unsubstituted heteroalkyl group. In some embodiments of the lipid of Formula CAT-I, R′ is an optionally substituted straight-chain heteroalkyl group. In some embodiments of the lipid of Formula CAT-I, R′ is a substituted straight-chain heteroalkyl group. In some embodiments of the lipid of Formula CAT-I, R^{L} is an unsubstituted straight-chain heteroalkyl group. In some embodiments of the lipid of Formula CAT-I, R^{L} is an optionally substituted branched heteroalkyl group. In some embodiments of the lipid of Formula CAT-I, R^{L} is a substituted branched heteroalkyl group. In some embodiments of the lipid of Formula CAT-I, R^{L} is an unsubstituted branched heteroalkyl group.

Exemplary unsubstituted heteroalkyl groups include, but are not limited to:

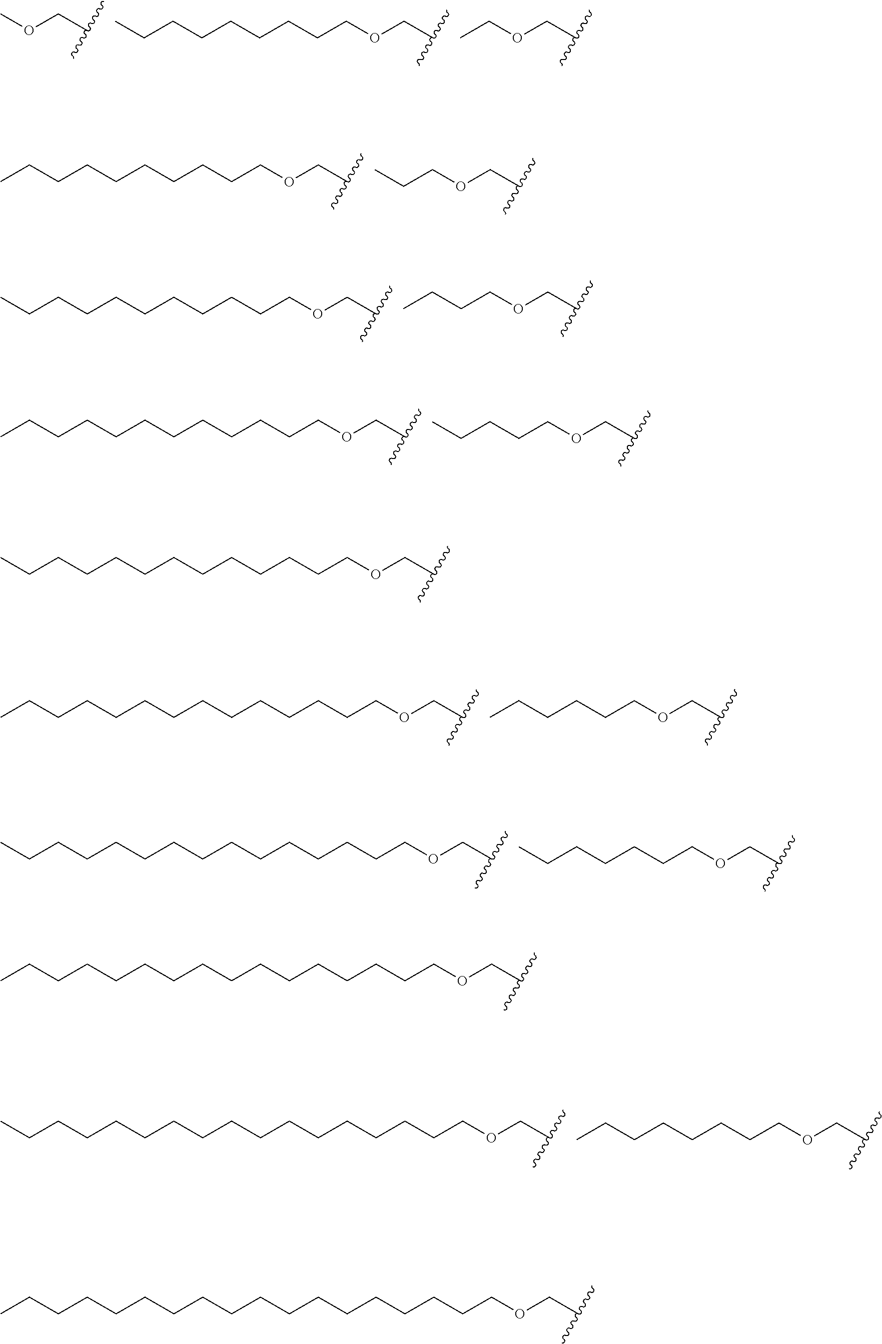

In some embodiments of the lipid of Formula CAT-I, R^{L} is optionally substituted heteroC₂₋₅₀alkenyl. In some embodiments of the lipid of Formula CAT-I, R^{L} is optionally substituted heteroC₂₋₃₀alkenyl. In some embodiments of the lipid of Formula CAT-I, R^{L} is optionally substituted heteroC₂₋₂₀alkenyl. In some embodiments of the lipid of Formula CAT-I, R^{L} is optionally substituted heteroC₂₋₁₅alkenyl. In some embodiments of the lipid of Formula CAT-I, R^{L} is optionally substituted heteroC₂₋₁₀alkenyl.

In some embodiments of the lipid of Formula CAT-I, R^{L} is optionally substituted heteroC₆₋₅₀alkenyl. In some embodiments of the lipid of Formula CAT-I, R^{L} is optionally substituted heteroC₆₋₃₀alkenyl. In some embodiments of the lipid of Formula CAT-I, R^{L} is optionally substituted heteroC₆₋₂₀alkenyl. In some embodiments of the lipid of Formula CAT-I, R^{L} is optionally substituted heteroC₆₋₁₅alkenyl. In some embodiments of the lipid of Formula CAT-I, R^{L} is optionally substituted heteroC₆₋₁₀alkenyl.

In some embodiments of the lipid of Formula CAT-I, for example, in any of the above embodiments, R^{L} is a substituted heteroalkenyl group. In some embodiments of the lipid of Formula CAT-I, R^{L} is an unsubstituted heteroalkenyl group. In some embodiments of the lipid of Formula CAT-I, R^{L} is an optionally substituted straight-chain heteroalkenyl group. In some embodiments of the lipid of Formula CAT-I, R^{L} is a substituted straight-chain heteroalkenyl group. In some embodiments of the lipid of Formula CAT-I, R^{L} is an unsubstituted straight-chain heteroalkenyl group. In some embodiments of the lipid of Formula CAT-I, R^{L} is an optionally substituted branched heteroalkenyl group. In some embodiments of the lipid of Formula CAT-I, R^{L} is a substituted branched heteroalkenyl group. In some embodiments of the lipid of Formula CAT-I, R^{L} is an unsubstituted branched heteroalkenyl group.

In some embodiments of the lipid of Formula CAT-I, R^{L} is optionally substituted heteroC₂₋₅₀alkynyl. In some embodiments of the lipid of Formula CAT-I, R^{L} is optionally substituted heteroC₂₋₃₀alkynyl. In some embodiments of the lipid of Formula CAT-I, R^{L} is optionally substituted heteroC₂₋₂₀alkynyl. In some embodiments of the lipid of Formula CAT-I, R^{L} is optionally substituted heteroC₂₋₁₅alkynyl. In some embodiments of the lipid of Formula CAT-I, R^{L} is optionally substituted heteroC₂₋₁₀alkynyl.

In some embodiments of the lipid of Formula CAT-I, R^{L} is optionally substituted heteroC₆₋₅₀alkynyl. In some embodiments of the lipid of Formula CAT-I, R^{L} is optionally substituted heteroC₆₋₃₀alkynyl. In some embodiments of the lipid of Formula CAT-I, R^{L} is optionally substituted heteroC₆₋₂₀alkynyl. In some embodiments of the lipid of Formula CAT-I, R^{L} is optionally substituted heteroC₆₋₁₅alkynyl. In some embodiments of the lipid of Formula CAT-I, R^{L} is optionally substituted heteroC₆₋₁₀alkynyl.

In some embodiments of the lipid of Formula CAT-I, for example, in any of the above embodiments, R^{L} is a substituted heteroalkynyl group. In some embodiments of the lipid of Formula CAT-I, R^{L} is an unsubstituted heteroalkynyl group. In some embodiments of the lipid of Formula CAT-I, R^{L} is an optionally substituted straight-chain heteroalkynyl group. In some embodiments of the lipid of Formula CAT-I, R^{L} is a substituted straight-chain heteroalkynyl group. In some embodiments of the lipid of Formula CAT-I, R^{L} is an unsubstituted straight-chain heteroalkynyl group. In some embodiments of the lipid of Formula CAT-I, R^{L} is an optionally substituted branched heteroalkynyl group. In some embodiments of the lipid of Formula CAT-I, R^{L} is a substituted branched heteroalkynyl group. In some embodiments of the lipid of Formula CAT-I, R^{L} is an unsubstituted branched heteroalkynyl group.

In some embodiments of the lipid of Formula CAT-I, R^{L} is a polymer. As used herein, a “polymer”, in some embodiments, refers to a compound comprised of at least 3 (e.g., at least 10, 20, 30, 40, 50, 60, 70, 80, 90, 100, etc.) repeating covalently bound structural units. The polymer is in certain embodiments biocompatible (i.e., non-toxic). Exemplary polymers include, but are not limited to, cellulose polymers (e.g., hydroxyethylcellulose, ethylcellulose, carboxymethylcellulose, methyl cellulose, hydroxypropylmethylcellulose (HPMC)), dextran polymers, polymaleic acid polymers, poly(acrylic acid) polymers, poly(vinylalcohol) polymers, polyvinylpyrrolidone (PVP) polymers, and polyethyleneglycol (PEG) polymers, and combinations thereof.

In some embodiments of the lipid of Formula CAT-I, R^{L} is a lipophilic, hydrophobic and/or non-polar group. In some embodiments of the lipid of Formula CAT-I, R^{L} is a lipophilic group. In some embodiments of the lipid of Formula CAT-I, R^{L} is a hydrophobic group. In some embodiments of the lipid of Formula CAT-I, R^{L} is a non-polar group.

In some embodiments of the lipid of Formula CAT-I, when an R^{L} group is depicted as bisecting a carbon-carbon bond, e.g., of the formula (i), it is understood that R^{L} may be bonded to either carbon.

Various combinations of the above embodiments of Formula CAT-I are contemplated herein.

In some embodiments, the lipid of Formula CAT-I has a structure according to Formula CAT-1c:

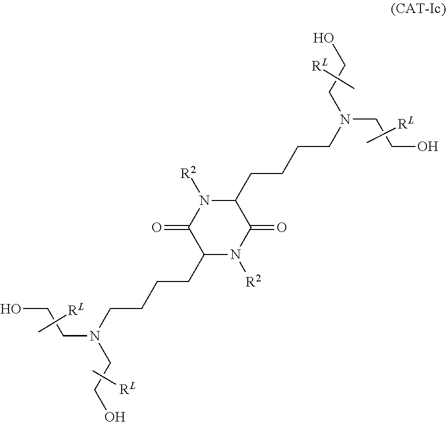

wherein each of R² and R^{L} is independently as defined above and described herein.

In some embodiments, the lipid of Formula CAT-I has a structure according to Formula CAT-Id:

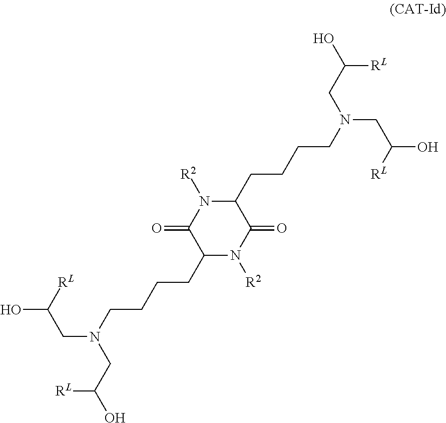

wherein each of R² and R^{L} is independently as defined above and described herein.

In some embodiments of the lipid of Formula CAT-I, CAT-Ia, CAT-Ib, CAT-Ic, or CAT-Id, R^{L} is C₁₋₂₀ alkyl or C₂₋₂₀ alkenyl. In some embodiments of the lipid of Formula CAT-I, CAT-Ia, CAT-Ib, CAT-Ic, or CAT-Id, R^{L} is C₆₋₂₀ alkyl or C₆₋₂₀ alkenyl.

In some embodiments, the lipid of Formula CAT-I is cKK-E10, having the following structure:

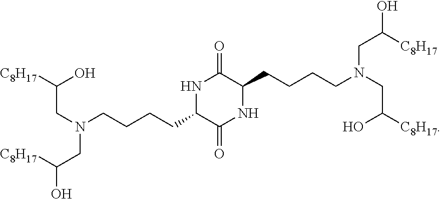

In some embodiments, the lipid of Formula CAT-I is OF-02, having the following structure:

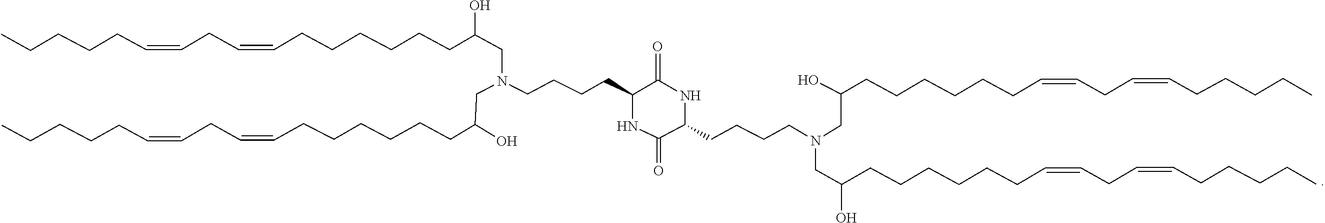 [SMILES: CCCCC/C=C\C/C=C\CCCCCCC(O)CN(CCCC[C@@H]1NC(=O)[C@@H](CCCCN(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC]

Additional examples of cationic lipids suitable for LNPs of the present disclosure are described in WO 2013063468, WO 2016205691, and WO 2013063468, each of which is incorporated by reference herein in its entirety.

In some embodiments, the cationic lipid has a structure according to Formula CAT-II:

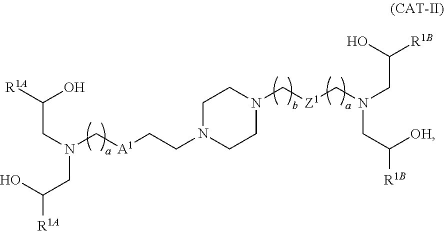

or a pharmaceutically acceptable salt thereof,
wherein:
A¹ is selected from

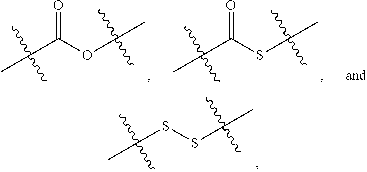

wherein the left hand side of each depicted structure is bound to the —(CH₂)a-;
Z¹ is selected from

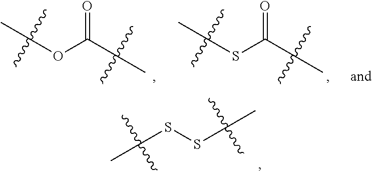

wherein the right hand side of each depicted structure is bound to the —(CH₂)a-;
R^{1A} and R^{1B} are each independently selected from optionally substituted alkyl, optionally substituted alkenyl, optionally substituted alkynyl, optionally substituted acyl, and —W¹—X¹—Y¹;
each W¹ is independently selected from optionally substituted alkyl and optionally substituted alkenyl;
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹,
each Y¹ is independently selected from hydrogen, —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to X¹;
b is 1, 2, 3, 4, or 5; and
each a is independently selected from 1, 2, 3, 4, 5, 6, 7, 8, 9, and 10.

In some embodiments, the lipid of Formula CAT-II has a structure according to Formula CAT-IIa:

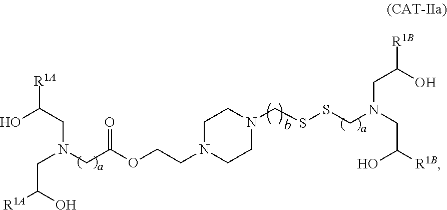

or a pharmaceutically acceptable salt thereof.

In some embodiments, the lipid of Formula CAT-II has a structure according to Formula CAT-IIb:

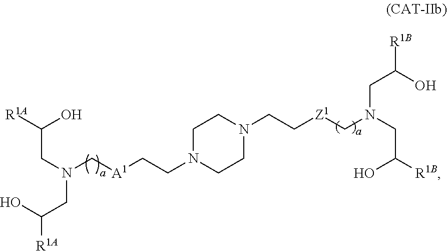

or a pharmaceutically acceptable salt thereof.

In some embodiments, the lipid of Formula CAT-II has a structure according to Formula CAT-IIc:

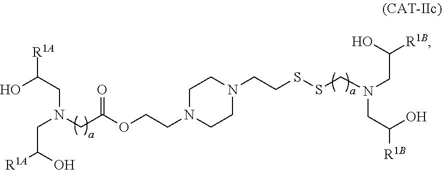

or a pharmaceutically acceptable salt thereof.

In some embodiments of the lipid of Formula CAT-II, A¹ and Z¹ are the same. In some embodiments of the lipid of Formula CAT-II, A¹ and Z¹ are different.

In some embodiments of the lipid of Formula CAT-II, A¹ is

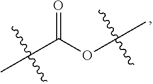 [SMILES: *OC(*)=O]

wherein the left hand side of the depicted structure is bound to the —(CH₂)a-. In some embodiments of the lipid of Formula CAT-II, A¹ is

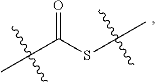 [SMILES: *SC(*)=O]

wherein the left hand side of the depicted structure is bound to the —(CH₂)a-. In some embodiments of the lipid of Formula CAT-II, A¹ is

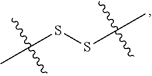 [SMILES: *SS*]

wherein the left hand side of the depicted structure is bound to the —(CH₂)a-.

In some embodiments of the lipid of Formula CAT-II, Z¹ is

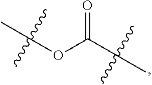 [SMILES: *OC(*)=O]

wherein the right hand side of the depicted structure is bound to the —(CH₂)a-. In some embodiments of the lipid of Formula CAT-II, Z¹ is

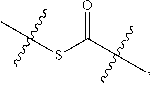 [SMILES: *SC(*)=O]

wherein the right hand side of the depicted structure is bound to the —(CH₂)a-. In some embodiments of the lipid of Formula CAT-II, Z¹ is

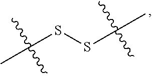 [SMILES: *SS*]

wherein the right hand side of the depicted structure is bound to the —(CH₂)a-.

In some embodiments of the lipid of Formula CAT-II, A¹ is

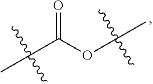 [SMILES: *OC(*)=O]

wherein the left hand side of the depicted structure is bound to the —(CH₂)a-, and Z¹ is

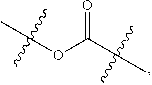 [SMILES: *OC(*)=O]

wherein the right hand side of the depicted structure is bound to the —(CH₂)a-.

In some embodiments of the lipid of Formula CAT-II, A¹ is

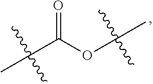 [SMILES: *OC(*)=O]

wherein the left hand side of the depicted structure is bound to the —(CH₂)a-, and Z¹ is

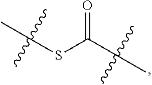 [SMILES: *SC(*)=O]

wherein the right hand side of the depicted structure is bound to the —(CH₂)a-.

In some embodiments of the lipid of Formula CAT-II, A¹ is

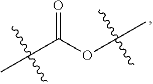 [SMILES: *OC(*)=O]

wherein the left hand side of the depicted structure is bound to the —(CH₂)a-, and Z¹ is

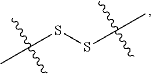 [SMILES: *SS*]

wherein the right hand side of the depicted structure is bound to the —(CH₂)a-.

In some embodiments of the lipid of Formula CAT-II, A¹ is

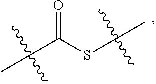 [SMILES: *SC(*)=O]

wherein the left hand side of the depicted structure is bound to the —(CH₂)a-, and Z¹ is

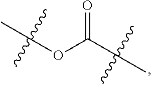 [SMILES: *OC(*)=O]

wherein the right hand side of the depicted structure is bound to the —(CH₂)a-.

In some embodiments of the lipid of Formula CAT-II, A¹ is

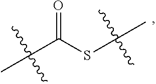 [SMILES: *SC(*)=O]

wherein the left hand side of the depicted structure is bound to the —(CH₂)a-, and Z¹ is

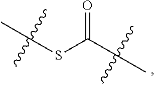 [SMILES: *SC(*)=O]

wherein the right hand side of the depicted structure is bound to the —(CH₂)a-.

In some embodiments of the lipid of Formula CAT-II, A¹ is

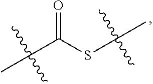 [SMILES: *SC(*)=O]

wherein the left hand side of the depicted structure is bound to the —(CH₂)a-, and Z¹ is

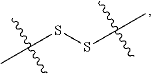 [SMILES: *SS*]

wherein the right hand side of the depicted structure is bound to the —(CH₂)a-.

In some embodiments of the lipid of Formula CAT-II, A¹ is

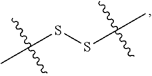 [SMILES: *SS*]

wherein the left hand side of the depicted structure is bound to the —(CH₂)a-, and Z¹ is

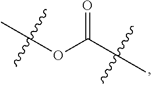 [SMILES: *OC(*)=O]

wherein the right hand side of the depicted structure is bound to the —(CH₂)a-.

In some embodiments of the lipid of Formula CAT-II, A¹ is

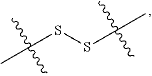 [SMILES: *SS*]

wherein the left hand side of the depicted structure is bound to the —(CH₂)a-, and Z¹ is

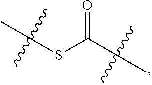 [SMILES: *SC(*)=O]

wherein the right hand side of the depicted structure is bound to the —(CH₂)a-.

In some embodiments of the lipid of Formula CAT-II, A¹ is

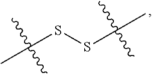 [SMILES: *SS*]

wherein the left hand side of the depicted structure is bound to the —(CH₂)a-, and Z¹ is

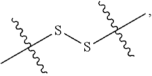 [SMILES: *SS*]

wherein the right hand side of the depicted structure is bound to the —(CH₂)a-.

In some embodiments of the lipid of Formula CAT-II, R^{1A} and R^{1B} are each independently selected from:

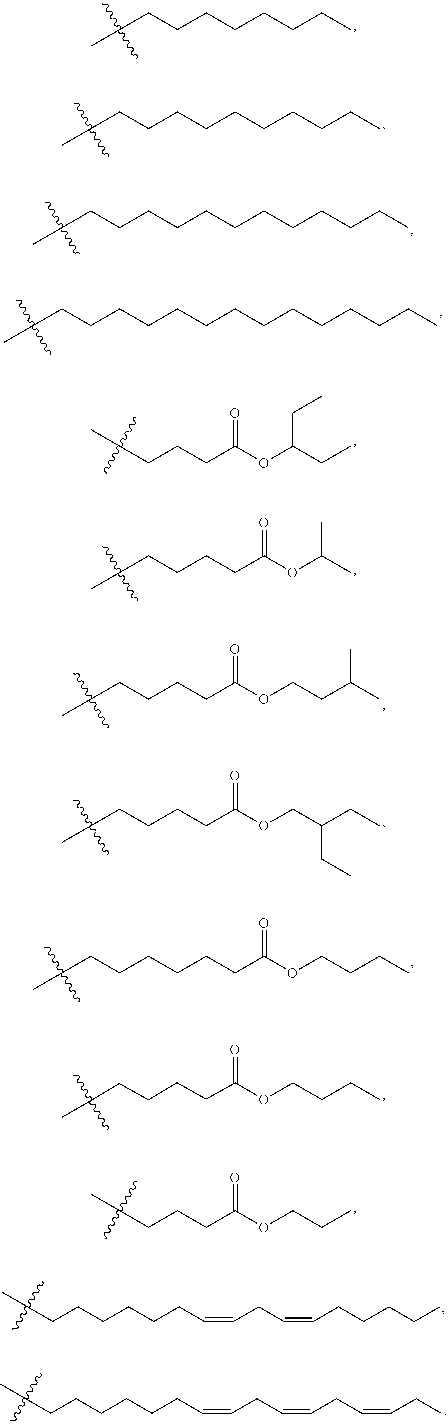

In some embodiments of the lipid of Formula CAT-II, each a is independently selected from 2, 3 and 4. In some embodiments of the lipid of Formula CAT-II, each a is the same. In some embodiments of the lipid of Formula CAT-II, each a is different.

In some embodiments of the lipid of Formula CAT-II, R^{1A} and RIB are —W¹—X¹—Y¹.

In some embodiments of the lipid of Formula CAT-II, W¹—X¹—Y¹ is defined as follows: each W¹ is independently selected from optionally substituted C₁₋₂₀ alkyl and optionally substituted C₂₋₂₀ alkenyl, each X¹ is independently selected from —*O—(C═O)-optionally substituted C₁₋₂₀ alkyl, —(*C═O)—O-optionally substituted C₁₋₂₀ alkyl, —*O—(C═O)-optionally substituted C₂₋₂₀ alkenyl, and —(*C═O)—O-optionally substituted C₂₋₂₀ alkenyl, wherein the atom marked with a * is connected to W¹, each Y¹ is independently selected from hydrogen, —*O—(C═O)-optionally substituted C₁₋₂₀ alkyl, —(*C═O)—O-optionally substituted C₁₋₂₀ alkyl, —*O—(C═O)-optionally substituted C₂₋₂₀ alkenyl, and —(*C═O)-optionally substituted C₂₋₂₀ alkenyl, wherein the atom marked with a * is connected to X¹.

In some embodiments of the lipid of Formula CAT-II, —W¹—X¹—Y¹; is defined as follows: each W¹ is independently selected from optionally substituted C_{A-B} alkyl and optionally substituted C_{C-D} alkenyl, each X¹ is independently selected from-*O—(C═O)-optionally substituted C_{A-B} alkyl —(*C═O)—O-optionally substituted C_{A-B} alkyl-*O—(C═O)-optionally substituted C_{C-D} alkenyl and —(*C═O)—O-optionally substituted C_{C-D} alkenyl wherein the atom marked with a * is connected to W¹, each Y¹ is independently selected from hydrogen, —*O—(C═O)-optionally substituted C_{A-B} alkyl, —(*C═O)—O-optionally substituted C_{A-B} alkyl, —*O—(C═O)-optionally substituted C_{C-D} alkenyl, and —(*C═O)—O-optionally substituted C_{C-D} alkenyl, wherein the atom marked with a * is connected to X¹.

In some embodiments of the lipid of Formula CAT-II, C_{A-B} is C₁₋₂₀ and C_{C-D} is C₂₋₂₀. In some embodiments C_{A-B} is C₁₋₁₅ and C_{C-D} is C₂₋₁₅. In some embodiments C_{A-B} is C₁₋₁₀ and C_{C-D} is C₂₋₁₀. In some embodiments C_{A-B} is C₃₋₁₅ and C_{C-D} is C₃₋₁₅. In some embodiments C_{A-B} is C₃₋₁₀ and C_{C-D} is C₃₋₁₀. In some embodiments C_{A-B} is C₃₋₈ and C_{C-D} is C₃₋₈.

In some embodiments of the lipid of Formula CAT-II, R^{1A} and R^{1B} are each independently selected from optionally substituted C₅₋₅₀ alkyl, optionally substituted C₅₋₅₀ alkenyl, optionally substituted C₅₋₅₀ alkynyl, optionally substituted C₅₋₅₀ acyl, and —W¹—X¹—Y¹, wherein —W¹—X¹—Y¹ is as defined herein.

In some embodiments of the lipid of Formula CAT-II, R^{1A} and R^{1B} are each independently selected from optionally substituted C₅₋₅₀ alkyl, optionally substituted C₅₋₅₀ alkenyl, optionally substituted C₅₋₅₀ alkynyl, and optionally substituted C₅₋₅₀ acyl.

In some embodiments of the lipid of Formula CAT-II, R^{1A} and R^{1B} are each independently selected from optionally substituted C₅₋₃₀ alkyl, optionally substituted C₅₋₃₀ alkenyl, optionally substituted C₅₋₃₀ alkynyl, optionally substituted C₅₋₃₀ acyl and —W¹—X¹—Y¹, wherein —W¹—X¹—Y¹ is as defined herein.

In some embodiments of the lipid of Formula CAT-II, R^{1A} and R^{1B} are each independently selected from optionally substituted C₅₋₃₀ alkyl, optionally substituted C₅₋₃₀ alkenyl, optionally substituted C₅₋₃₀ alkynyl, and optionally substituted C₅₋₃₀ acyl.

In some embodiments of the lipid of Formula CAT-II, R^{1A} and R^{1B} are each: independently selected from optionally substituted C₅₋₂₀ alkyl, optionally substituted C₅₋₂₀ alkenyl, optionally substituted C₅₋₂₀ alkynyl, optionally substituted C₅₋₂₀ acyl, and —W¹—X¹—Y¹, wherein —W¹—X¹—Y¹.

In some embodiments of the lipid of Formula CAT-II, R^{1A} and R^{1B} are each independently selected from optionally substituted C₅₋₂₀ alkyl, optionally substituted C₅₋₂₀ alkenyl, optionally substituted C₅₋₂₀ alkynyl, and optionally substituted C₅₋₂₀ acyl.

In some embodiments of the lipid of Formula CAT-II, R^{1A} and R^{1B} are each independently optionally substituted C₅₋₅₀ alkyl. In some embodiments of the lipid of Formula CAT-II, R^{1A} and R^{1B} are each independently optionally substituted C₅₋₃₀ alkyl. In some embodiments of the lipid of Formula CAT-II, R^{1A} and R^{1B} are each independently optionally substituted C₅₋₂₀ alkyl. In some embodiments of the lipid of Formula CAT-II, R^{1A} and R^{1B} are each independently optionally substituted C₅₋₁₅ alkyl.

In some embodiments of the lipid of Formula CAT-II, R^{1A} and R^{1B} are each independently C₅₋₅₀ alkyl. In some embodiments of the lipid of Formula CAT-II, R^{1A} and R^{1B} are each independently C₅₋₅₀ alkyl. In some embodiments of the lipid of Formula CAT-II, R^{1A} and R^{1B} are each independently C₅₋₂₀ alkyl. In some embodiments of the lipid of Formula CAT-II, R^{1A} and R^{1B} are each independently C₅₋₁₅ alkyl.

In some embodiments of the lipid of Formula CAT-II, R^{1A} and R^{1B} are each independently optionally substituted C₅₋₅₀ alkenyl. In some embodiments of the lipid of Formula CAT-II, R^{1A} and R^{1B} are each independently optionally substituted C₅₋₃₀ alkenyl. In some embodiments of the lipid of Formula CAT-II, R^{1A} and R^{1B} are each independently optionally substituted C₅₋₂₀ alkenyl. In some embodiments of the lipid of Formula CAT-II, R^{1A} and R^{1B} are each independently optionally substituted C₅₋₁₅ alkenyl.

In some embodiments of the lipid of Formula CAT-II, R^{1A} and R^{1B} are each independently C₅₋₅₀ alkenyl. In some embodiments of the lipid of Formula CAT-II, R^{1A} and R^{1B} are each independently C₅₋₃₀ alkenyl. In some embodiments of the lipid of Formula CAT-II, R^{1A} and R^{1B} are each independently C₅₋₂₀ alkenyl. In some embodiments of the lipid of Formula CAT-I, R^{1A} and R^{1B} are each independently C₅₋₁₅ alkenyl.

In some embodiments of the lipid of Formula CAT-II, RIA and R^{1B} are each independently optionally substituted C₅₋₅₀ alkynyl. In some embodiments of the lipid of Formula CAT-II, R^{1A} and R^{1B} are each independently optionally substituted C₅₋₃₀ alkynyl. In some embodiments of the lipid of Formula CAT-II, R^{1A} and R^{1B} are each independently optionally substituted C₅₋₂₀ alkynyl. In some embodiments of the lipid of Formula CAT-II, R^{1A} and R^{1B} are each independently optionally substituted C₅₋₁₅ alkynyl.

In some embodiments of the lipid of Formula CAT-II, R^{1A} and R^{1B} are each independently C₅₋₅₀ alkynyl. In some embodiments of the lipid of Formula CAT-II, R^{1A} and R^{1B} are each independently C₅₋₃₀ alkynyl. In some embodiments of the lipid of Formula CAT-II, R^{1A} and R^{1B} are each independently C₅₋₂₀ alkynyl. In some embodiments of the lipid of Formula CAT-II, R^{1A} and R^{1B} are each independently C₅₋₁₅ alkynyl.

In some embodiments of the lipid of Formula CAT-II, R^{1A} and R^{1B} are not optionally substituted. In some embodiments of the lipid of Formula CAT-II, each R^{1A} is the same. In some embodiments of the lipid of Formula CAT-II, each R^{1A} is different. In some embodiments of the lipid of Formula CAT-II, each R^{1B} is the same. In some embodiments of the lipid of Formula CAT-II, each R^{1B} is different. In some embodiments of the lipid of Formula CAT-II, R^{1A} and R^{1B} are the same. In some embodiments of the lipid of Formula CAT-II, R^{1A} and R^{1B} are different.

In some embodiments, the lipid of Formula CAT-II is GL-HEPES-E3-E10-DS-3-E18-1 (2-(4-(2-((3-(Bis((Z)-2-hydroxyoctadec-9-en-1-yl)amino)propyl)disulfaneyl)ethyl)piperazin-1-yl)ethyl 4-(bis(2-hydroxydecyl)amino)butanoate), having the following structure:

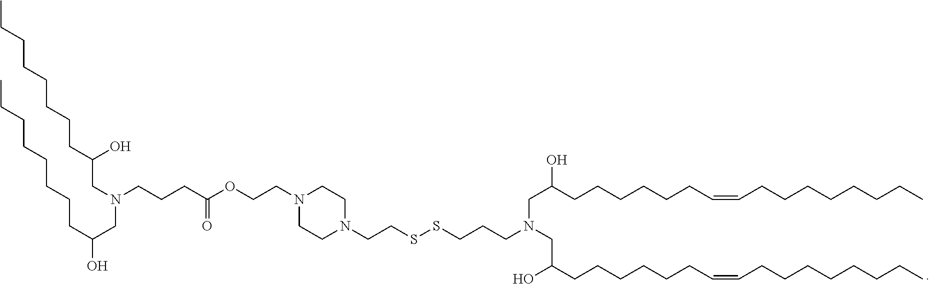 [SMILES: CCCCCCCC/C=C\CCCCCCC(O)CN(CCCSSCCN1CCN(CCOC(=O)CCCN(CC(O)CCCCCCCC)CC(O)CCCCCCCC)CC1)CC(O)CCCCCC/C=C\CCCCCCCC]

In some embodiments, the lipid of Formula CAT-II is 2-(4-(3-((4-(bis((Z)-2-hydroxyoctadec-9-en-1-yl)amino)butyl)disulfaneyl)propyl)piperazin-1-yl)ethyl 4-(bis(2-hydroxydecyl)amino)butanoate, having the following structure:

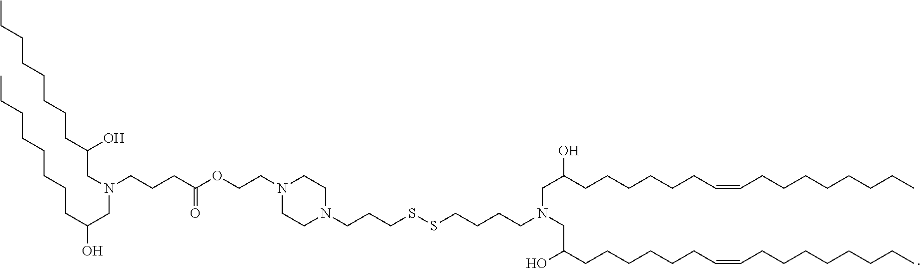 [SMILES: CCCCCCCC/C=C\CCCCCCC(O)CN(CCCCSSCCCN1CCN(CCOC(=O)CCCN(CC(O)CCCCCCCC)CC(O)CCCCCCCC)CC1)CC(O)CCCCCC/C=C\CCCCCCCC]

In some embodiments, the lipid of Formula CAT-II is GL-HEPES-E3-E12-DS-4-E10, (2-(4-(2-((3-(bis(2-hydroxydecyl)amino)butyl)disulfaneyl)ethyl)piperazin-1-yl)ethyl 4-(bis(2-hydroxydodecyl)amino)butanoate), having the following structure:

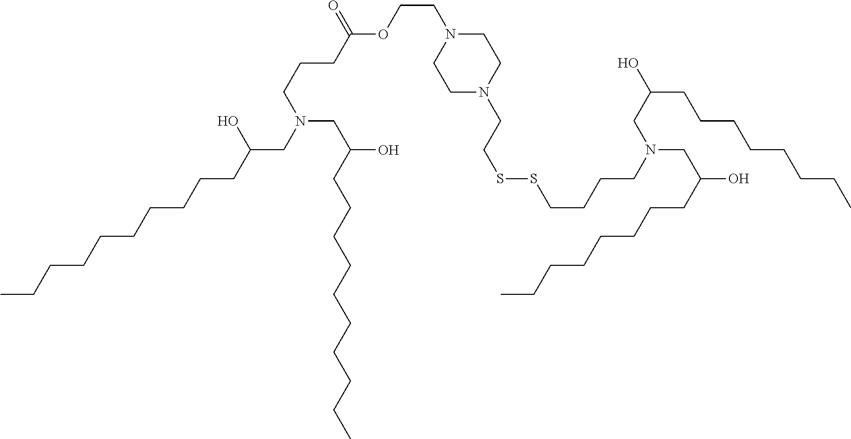 [SMILES: CCCCCCCCCCC(O)CN(CCCC(=O)OCCN1CCN(CCSSCCCCN(CC(O)CCCCCCCC)CC(O)CCCCCCCC)CC1)CC(O)CCCCCCCCCC]

In some embodiments, the lipid of Formula CAT-II is GL-HEPES-E3-E12-DS-3-E14 (2-(4-(2-((3-(Bis(2-hydroxytetradecyl)amino)propyl)disulfaneyl)ethyl)piperazin-1-yl)ethyl 4-(bis(2-hydroxydodecyl)amino)butanoate), having the following structure:

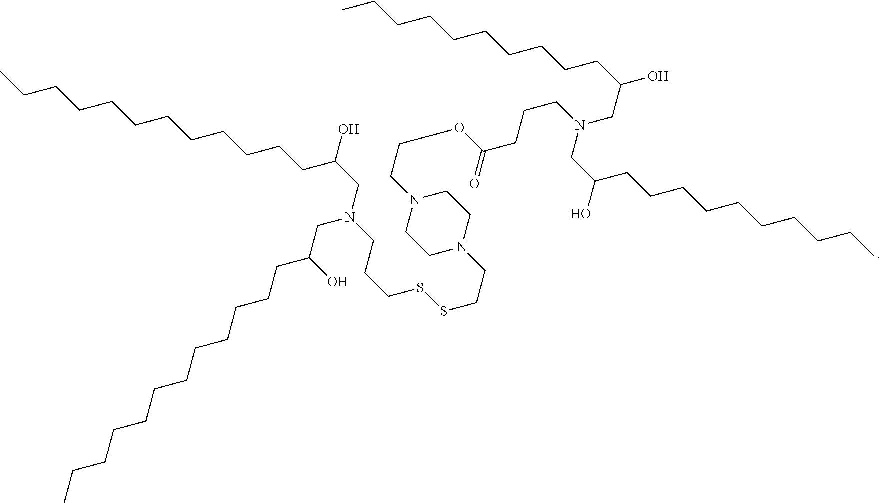 [SMILES: CCCCCCCCCCCCC(O)CN(CCCSSCCN1CCN(CCOC(=O)CCCN(CC(O)CCCCCCCCCC)CC(O)CCCCCCCCCC)CC1)CC(O)CCCCCCCCCCCC]

Additional examples of cationic lipids suitable for LNPs of the present disclosure are described in WO 2022221688, which is incorporated by reference herein in its entirety.

Other cationic lipids that can be used include those described, for example, in WO 2016176330, WO 2017049245, and WO 2017075531.

Accordingly, in some embodiments, the cationic lipid has a structure according to Formula CAT-III:

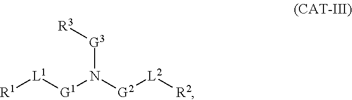

or a pharmaceutically acceptable salt thereof, wherein:
one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, —NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂alkyl;
R⁵ is H or C₁-C₆ alkyl; and
x is 0, 1 or 2.

Accordingly, in some embodiments, the cationic lipid has a structure according to Formula CAT-IV:

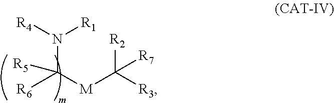

or a pharmaceutically acceptable salt thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H,
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, the cationic lipid has a structure according to Formula CAT-V:

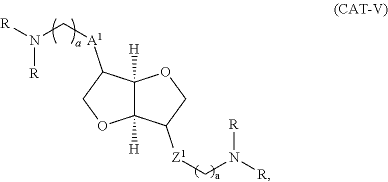

or a pharmaceutically acceptable salt thereof wherein:
A¹ is selected from —C(═O)O—, —C(═O)S—, —C(═O)NH—, —OC(═O)O—, —OC(═O)NH—, —NHC(═O)O—, —SC(═O)NH—, —OCH₂CH₂O—, —OCH₂O—, —OCH(CH₃)O—, —S— and —S—S—, wherein the left hand side of each recited structure is bound to the —(CH₂)ₐ—;
Z¹ is selected from —OC(═O)—, —SC(═O)—, —NHC(═O)—, —OC(═O)O—, —NHC(═O)O—, —OC(═O)NH—, —NHC(═O)S—, —OCH₂CH₂O—, —OCH₂O—, —OCH(CH₃)O—, —S— and —S—S—, wherein the right hand side of each recited structure is bound to the —(CH₂)ₐ—;
each R is independently selected from:

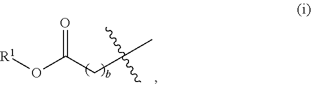

wherein each R¹ is independently selected from optionally substituted alkyl, optionally substituted alkenyl, and optionally substituted alkynyl;

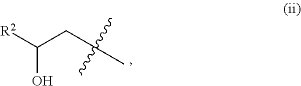

wherein each R² is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from-*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;

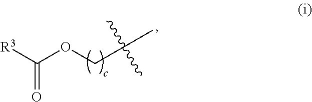

wherein each R³ is independently selected from optionally substituted alkyl, optionally substituted alkenyl, and optionally substituted alkynyl; and

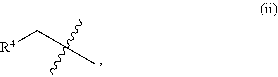

wherein each R⁴ is independently selected from optionally substituted cycloalkyl or optionally substituted heterocycloalkyl;
wherein at least three R are independently selected from

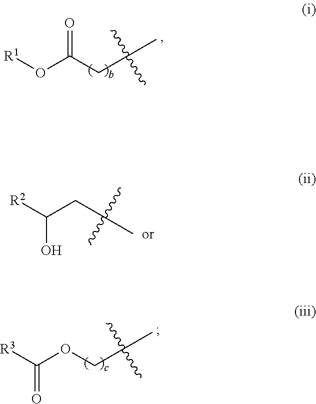

each a is independently selected from 2, 3, 4, and 5;
each b is independently selected from 2, 3, 4, 5, 6, 7, 8, 9 and 10; and
each c is independently selected from 2, 3, 4, 5, 6, 7, 8, 9 and 10.

In some embodiments, the lipid of Formula CAT-V has a structure according to Formula CAT-Va:

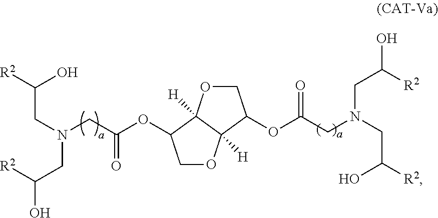

or a pharmaceutically acceptable salt thereof.

In some embodiments, the lipid of Formula CAT-V has a structure according to Formula CAT-Vb:

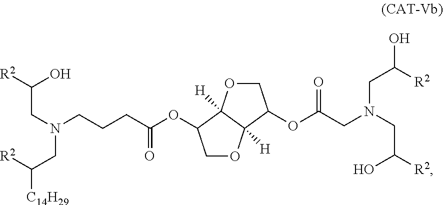

or a pharmaceutically acceptable salt thereof.

In some embodiments of the lipid of Formula V. Formula Va, or Formula Vb, R² is optionally substituted alkyl or —W¹—X¹. In some embodiments of the lipid of Formula V, Formula Va, or Formula Vb, R² is optionally substituted alkyl. In some embodiments of the lipid of Formula V, Formula Va, or Formula Vb, R² is alkyl. In some embodiments of the lipid of Formula V, Formula Va, or Formula Vb, R² is —W¹—X¹.

In some embodiments of the lipid of Formula V, Formula Va, or Formula Vb, each W¹ is independently optionally substituted alkylene. In some embodiments of the lipid of Formula V, Formula Va, or Formula Vb, each W¹ is independently alkylene.

In some embodiments of the lipid of Formula V, Formula Va, or Formula Vb, each X¹ is independently selected from —*O—(C═O)-optionally substituted alkyl and —(*C═O)—O-optionally substituted alkyl, wherein the atom marked with a * is connected to W¹. In some embodiments of the lipid of Formula V, Formula Va, or Formula Vb, each X¹ is independently selected from —*O—(C═O)-alkyl and —(*C═O)—O-alkyl, wherein the atom marked with a * is connected to W¹.

In some embodiments, the lipid of Formula CAT-V is IM-001 ((3R,3aR,6R,6aR)-hexahydrofuro[3,2-b]furan-3,6-diyl bis(4-(bis(2-hydroxydodecyl)amino)butanoate)), having the following structure:

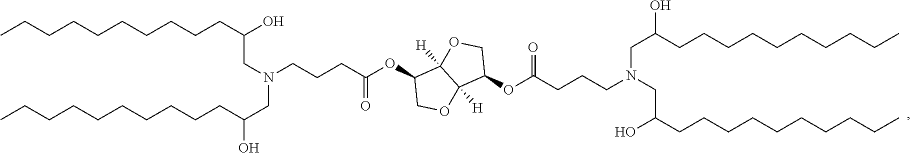 [SMILES: CCCCCCCCCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCCCCCC]

or a pharmaceutically acceptable salt thereof.

In some embodiments, the lipid of Formula CAT-V is IS-001 ((3R,3aR,6S,6aR)-hexahydrofuro[3,2-b]furan-3,6-diyl bis(4-(bis(2-hydroxydodecyl)amino)butanoate)), having the following structure:

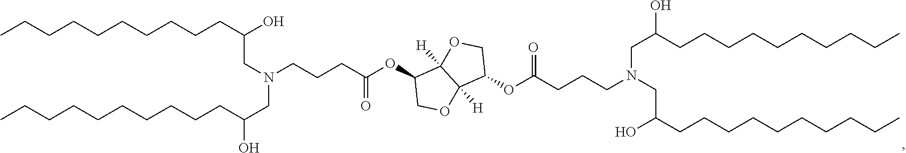 [SMILES: CCCCCCCCCCC(O)CN(CCCC(=O)O[C@H]1CO[C@H]2[C@@H]1OC[C@H]2OC(=O)CCCN(CC(O)CCCCCCCCCC)CC(O)CCCCCCCCCC)CC(O)CCCCCCCCCC]

or a pharmaceutically acceptable salt thereof.

In some embodiments, the cationic lipid has a structure according to Formula CAT-VI:

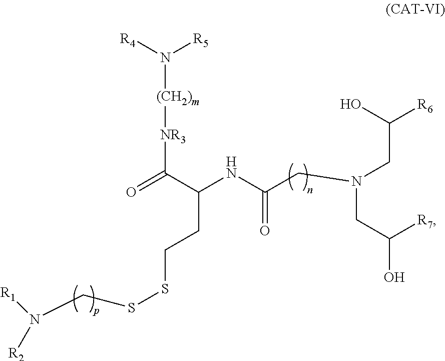

or a pharmaceutically acceptable salt thereof, wherein:
m is an integer chosen between 1 to 6; for example 2 to 4;
n is an integer chosen between 1 to 6; for example 2 to 4;
p is an integer chosen between 1 to 6; for example 2 to 4;
R¹ and R² are independently selected from the group consisting of linear or branched (C₁-C₃₀) alkyl and linear or branched (C₂-C₃₀) alkenyl,
wherein each alkyl and alkenyl are optionally interrupted by one or more groups selected from —C═O—, —C═OO— and —O—, and/or
wherein each alkyl and alkenyl are optionally substituted by one or more substituents selected from —OR, —CN—, —(C₁-C₆) alkyl-OH, —CF₃, —NO₂, —COOR, —SR, halogen atoms and —NRR′;
R³ is selected from the group consisting of H, (C₁-C₆) alkyl optionally substituted by one or more substituents selected from —OR, —CN—, —(C₁-C₆) alkyl-OH, —CF₃, —NO₂, —COOR, —SR, halogen atoms and —NRR′;
R⁴ and R⁵ are independently selected from the group consisting of linear or branched (C₁-C₆) alkyl and linear or branched (C₂-C₆) alkenyl, wherein each alkyl or alkenyl is optionally substituted with one or more of substituents selected from the group consisting of —OR, —CN—, —(C₁-C₆) alkyl-OH, —CF₃, —NO₂, —COOR, —SR, halogen atoms and —NRR′;
or R⁴ and R⁵ together with the N atom to which they are attached form;
a 5 to 6 membered cycloalkyl or heterocycle comprising 1 to 4 heteroatoms selected from O, N and S, or
a 5 to 6 membered aryl or heteroaryl comprising 1 to 4 heteroatoms selected from O, N and S,
wherein said cycloalkyl, heterocycle, aryl or heteroaryl is optionally substituted with one or more substituents selected from —OR, —CN—, —(C₁-C₆) alkyl-OH, —CF₃, —NO₂, —COOR, —SR, halogen atoms and —NRR′;
R⁶ and R⁷ are independently selected from the group consisting of linear or branched (C₁-C₃₀) alkyl and linear or branched (C₂-C₃₀) alkenyl,
wherein each alkyl and alkenyl are optionally interrupted by one or more groups selected from —C═O—, —C═OO— and —O—, and/or
each alkyl and alkenyl are optionally substituted by one or more substituents selected from —OR, —CN—, —C₁-C₆ alkyl-OH, —CF₃, —NO₂, —COOR, —SR, halogen atoms and —NRR′; and
R, R′ are independently selected from H and (C₁-C₆) alkyl.

In some embodiments of the lipid of Formula VI, R¹ and R² are independently selected from the group consisting of linear or branched (C₅-C₃₀) alkyl and linear or branched (C₂-C₃₀) alkenyl, wherein each alkyl and alkenyl are optionally substituted with one —OH group. In some embodiments of the lipid of Formula VI, R¹ and R² are independently linear or branched (C₅-C₃₀) alkyl substituted with one —OH group. In some embodiments of the lipid of Formula VI, R¹ and R² are independently linear (C₅-C₃₀) alkyl substituted with one —OH group.

In some embodiments of the lipid of Formula VI, R⁴ and R⁵ together with the N atom to which they are attached form: a 5 to 6 membered cycloalkyl or heterocycle comprising 1 to 4 heteroatoms selected from O, N and S, or a 5 to 6 membered heteroaryl comprising 1 to 4 heteroatoms selected from O, N and S. In some embodiments of the lipid of Formula VI, R⁴ and R⁵ together with the N atom to which they are attached form a 5 to 6 membered heteroaryl comprising 1 to 4 heteroatoms selected from O, N and S. In some embodiments of the lipid of Formula VI, R⁴ and R⁵ together with the N atom to which they are attached form an imidazolyl group.

In some embodiments of the lipid of Formula VI, R⁶ and R⁷ are independently selected from the group consisting of linear or branched (C₁-C₃₀) alkyl and linear or branched (C₂-C₃₀) alkenyl. In some embodiments of the lipid of Formula VI, R⁶ and R⁷ are independently linear or branched (C₁-C₃₀) alkyl. In some embodiments of the lipid of Formula VI, R⁶ and R⁷ are independently linear (C₁-C₃₀) alkyl.

In some embodiments, the lipid of Formula CAT-VI is A2H7iiT6 (N-(1-((3-(1H-imidazol-1-yl)propyl)amino)-4-((4-(bis(2-hydroxytetradecyl)amino)butyl)disulfaneyl)-1-oxobutan-2-yl)-5-(bis(2-hydroxydecyl)amino)pentanamide), having the following structure:

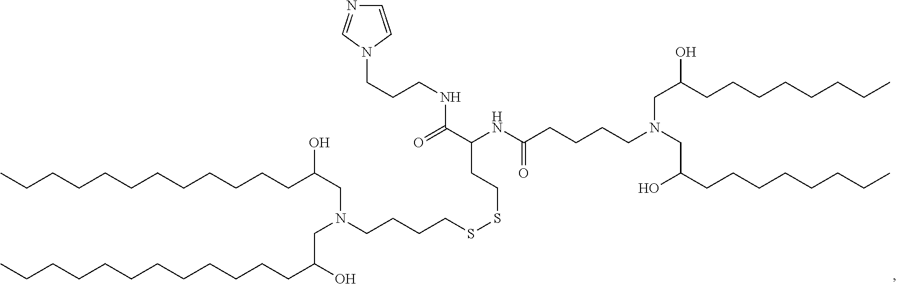 [SMILES: CCCCCCCCCCCCC(O)CN(CCCCSSCCC(NC(=O)CCCCN(CC(O)CCCCCCCC)CC(O)CCCCCCCC)C(=O)NCCCn1ccnc1)CC(O)CCCCCCCCCCCC]

or a pharmaceutically acceptable salt thereof.

In some embodiments, the cationic lipid is MC3, having the following structure:

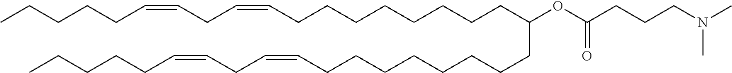 [SMILES: CCCCC/C=C\C/C=C\CCCCCCCCC(CCCCCCCC/C=C\C/C=C\CCCCC)OC(=O)CCCN(C)C]

In some embodiments, the cationic lipid is SM-102 (9-heptadecanyl 8-{(2-hydroxyethyl)[6-oxo-6-(undecyloxy)hexyl]amino}octanoate), having the following structure:

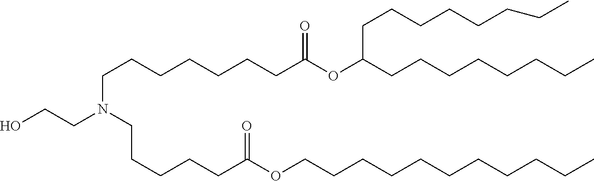 [SMILES: CCCCCCCCCCCOC(=O)CCCCCN(CCO)CCCCCCCC(=O)OC(CCCCCCCC)CCCCCCCC]

In some embodiments, the cationic lipid is ALC-0315 [(4-hydroxybutyl)azanediyl]di(hexane-6,1-diyl) bis(2-hexyldecanoate), having the following structure:

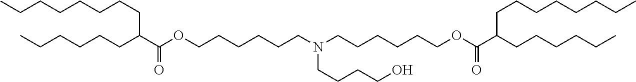 [SMILES: CCCCCCCCC(CCCCCC)C(=O)OCCCCCCN(CCCCO)CCCCCCOC(=O)C(CCCCCC)CCCCCCCC]

In some embodiments, the cationic lipid is cOrn-EE1, having the following structure:

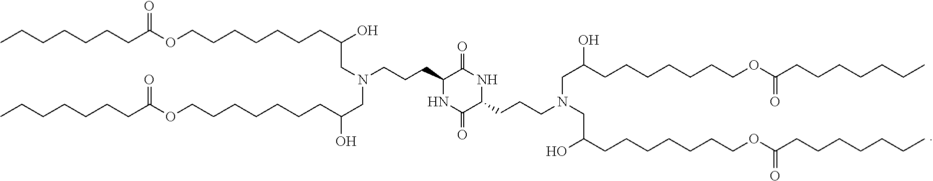 [SMILES: CCCCCCCC(=O)OCCCCCCCC(O)CN(CCC[C@@H]1NC(=O)[C@@H](CCCN(CC(O)CCCCCCCOC(=O)CCCCCCC)CC(O)CCCCCCCOC(=O)CCCCCCC)NC1=O)CC(O)CCCCCCCOC(=O)CCCCCCC]

In some embodiments, the cationic lipid is BAL-005 (bis(3-(bis(2-hydroxydodecyl)amino)propyl) 2,2′-(methylazanediyl)diacetate), having the following structure:

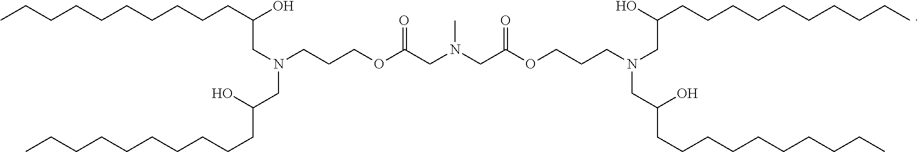 [SMILES: CCCCCCCCCCC(O)CN(CCCOC(=O)CN(C)CC(=O)OCCCN(CC(O)CCCCCCCCCC)CC(O)CCCCCCCCCC)CC(O)CCCCCCCCCC]

In some embodiments, the cationic lipid is BAL-020 (bis(3-(bis(2-hydroxydodecyl)amino)propyl) 3-hydroxy-3-methylpentanedioate), having the following structure:

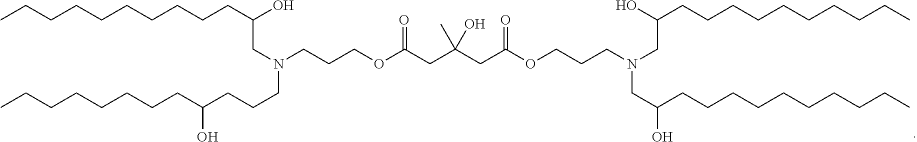 [SMILES: CCCCCCCCCCC(O)CN(CCCOC(=O)CC(C)(O)CC(=O)OCCCN(CC(O)CCCCCCCCCC)CC(O)CCCCCCCCCC)CCCC(O)CCCCCCCC]

In some embodiments, the cationic lipid is HEP-E4-E12 [(2,5-dimethylpiperazine-1,4-diyl)bis(ethane-2,1-diyl) bis(5-(bis(2-hydroxydodecyl)amino)pentanoate)], having the following structure:

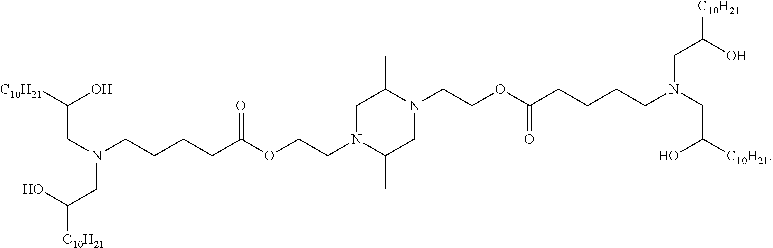

In some embodiments, the cationic lipid is TL1-12D-DMA (tris(5-(octanoyloxy)pentyl) 2-((3-(dimethylamino)propanoyl)oxy)propane-1,2,3-tricarboxylate), having the following structure:

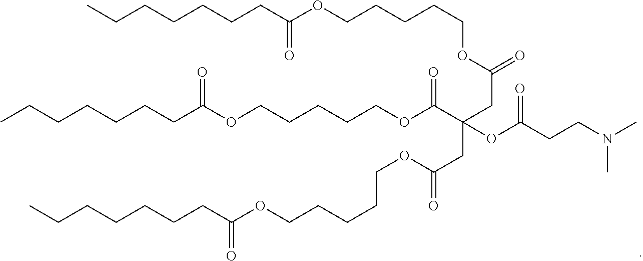 [SMILES: CCCCCCCC(=O)OCCCCCOC(=O)CC(CC(=O)OCCCCCOC(=O)CCCCCCC)(OC(=O)CCN(C)C)C(=O)OCCCCCOC(=O)CCCCCCC]

In some embodiments, the cationic lipid may be selected from the group comprising cKK-E10; OF-02; [(6Z,9Z,28Z,31Z)-heptatriaconta-6,9,28,31-tetraen-19-yl]4-(dimethylamino)butanoate (D-Lin-MC3-DMA); 2,2-dilinoleyl-4-dimethylaminoethyl-[1,3]-dioxolane (DLin-KC2-DMA); 1,2-dilinoleyloxy-N,N-dimethyl-3-aminopropane (DLin-DMA); di((Z)-non-2-en-1-yl) 9-((4-(dimethylamino)butanoyl)oxy)heptadecanedioate (L319); 9-heptadecanyl 8-{(2-hydroxyethyl)[6-oxo-6-(undecyloxy)hexyl]amino}octanoate (SM-102); [(4-hydroxybutyl)azanediyl]di(hexane-6,1-diyl) bis(2-hexyldecanoate) (ALC-0315); [3-(dimethylamino)-2-[(Z)-octadec-9-enoyl]oxypropyl](Z)-octadec-9-enoate (DODAP); 2,5-bis(3-aminopropylamino)-N-[2-[di(heptadecyl)amino]-2-oxoethyl]pentanamide (DOGS); [(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl]N-[2-(dimethylamino)ethyl]carbamate (DC-Chol); tetrakis(8-methylnonyl) 3,3′,3″,3′″-(((methylazanediyl) bis(propane-3,1 diyl))bis (azanetriyl))tetrapropionate (306Oi10); decyl (2-(dioctylammonio)ethyl) phosphate (9A1P9); ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18); bis(2-(dodecyldisulfanyl)ethyl) 3,3′-((3-methyl-9-oxo-10-oxa-13,14-dithia-3,6-diazahexacosyl)azanediyl)dipropionate (BAME-016B); 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol) (C12-200); 3,6-bis(4-(bis(2-hydroxydodecyl)amino)butyl)piperazine-2,5-dione (cKK-E12); hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate (FTT5); (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate) (OF-Deg-Lin); TT3; N¹,N³,N⁵-tris(3-(didodecylamino)propyl)benzene-1,3,5-tricarboxamide; N1-[2-((1S)-1-[(3-aminopropyl)amino]-4-[di(3-aminopropyl)amino]butylcarboxamido)ethyl]-3,4-di[oleyloxy]-benzamide (MVL5); heptadecan-9-yl 8-((2-hydroxyethyl)(8-(nonyloxy)-8-oxooctyl)amino)octanoate (Lipid 5); GL-HEPES-E3-E10-DS-3-E18-1; GL-HEPES-E3-E12-DS-4-E10; GL-HEPES-E3-E12-DS-3-E14; and combinations thereof.

In some embodiments, the cationic lipid is biodegradable.

In some embodiments, the cationic lipid is not biodegradable.

In some embodiments, the cationic lipid is cleavable.

In some embodiments, the cationic lipid is not cleavable.

Cationic lipids are described in further detail in Dong et al. (PNAS. 111(11):3955-60, 2014); Fenton et al. (Adv Mater. 28:2939. 2016); U.S. Pat. Nos. 9,512,073; 10,201,618; EP 22307007.9; EP 22307007.9; WO 2022/221688; WO 2022/066916; and WO 2022/0257716, each of which is incorporated herein by reference.

### Glycerides and Acylglycols

Glycerides or acylglycols are hydrophobic ester compounds formed from glycerol and fatty acids that facilitate delivery of the LNPs. Accordingly, LNPs of the present disclosure that include a glyceride or an acylglycol may exhibit improved delivery efficiency compared to LNPs that don't include a glyceride or an acylglycol.

In some embodiments, the glyceride or acylglycol is a monoglyceride, a diglyceride, a triglyceride or a diacylglycol. In some embodiments, the glyceride or is a monoglyceride, a diglyceride, or a triglyceride. In some embodiments, the glyceride is a monoglyceride. In some embodiments, the glyceride is a diglyceride. In some embodiments, a glyceride is a triglyceride. In some embodiments, the acylglycol is a diacylglycol.

In some embodiments, the glyceride or acylglycol has a structure according to Formula I or Formula II:

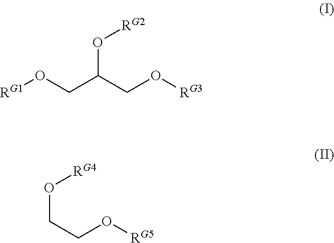

wherein:
R^{G1}, R^{G2}, and R^{G3} are each independently H, —C₍₁₋₂₅₎alkyl, —C₍₁₋₂₅₎alkenyl, —C(O)C₍₁₋₂₅₎alkyl, or —C(O)C₍₁₋₂₅₎alkenyl, wherein the —C₍₁₋₂₅₎alkyl, —C₍₁₋₂₅₎alkenyl, —C(O)C₍₁₋₂₅₎alkyl, and —C(O)C₍₁₋₂₅₎alkenyl are optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl;
provided that no more than two of R^{G1}, R^{G2} and R^{G3} are H;
R^{G4} is-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl;
R^{G5} is H, —C₍₁₋₂₅₎alkyl, —C₍₁₋₂₅₎alkenyl, —C(O)C₍₁₋₂₅₎alkyl, or —C(O)C₍₁₋₂₅₎alkenyl, wherein the —C₍₁₋₂₅₎alkyl, —C₍₁₋₂₅₎alkenyl, —C(O)C₍₁₋₂₅₎alkyl, and —C(O)C₍₁₋₂₅₎alkenyl are optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅)alkyl, and —C(O)C₍₁₋₂₅₎alkenyl.

In some embodiments, the glyceride or acylglycol has a structure according to Formula I or Formula II, wherein:
R^{G1}, R^{G2}, and R^{G3} are each independently H, —C₍₁₋₂₅₎alkyl, —C₍₁₋₂₅₎alkenyl, —C(O)C₍₁₋₂₅₎alkyl, or —C(O)C₍₁₋₂₅₎alkenyl;
provided that no more than two of R^{G1}, R^{G2} and R^{G3} are H;
R^{G4} is-C₍₁₋₂₅₎alkyl, —C₍₁₋₂₅₎alkenyl, —C(O)C₍₁₋₂₅₎alkyl, or —C(O)C₍₁₋₂₅₎alkenyl;
R^{G5} is H, —C₍₁₋₂₅₎alkyl, —C₍₁₋₂₅₎alkenyl, —C(O)C₍₁₋₂₅₎alkyl, or —C(O)C₍₁₋₂₅₎alkenyl.

In some embodiments, the glyceride or acylglycol has a structure according to Formula I:

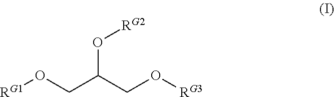

wherein:
R^{G1}, R^{G2}, and R^{G3} are each independently H, —C₍₁₋₂₅₎alkyl, —C₍₁₋₂₅₎alkenyl, —C(O)C₍₁₋₂₅₎alkyl, or —C(O)C₍₁₋₂₅₎alkenyl, wherein the —C₍₁₋₂₅₎alkyl, —C₍₁₋₂₅₎alkenyl, —C(O)C₍₁₋₂₅₎alkyl, and —C(O)C₍₁₋₂₅₎alkenyl are optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl; and
provided that no more than two of R^{G1}, R^{G2} and R^{G3} are H.

In some embodiments, the glyceride or acylglycol has a structure according to Formula I:

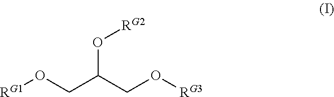

wherein:
R^{G1}, R^{G2}, and R^{G3} are each independently H, —C₍₁₋₂₅₎alkyl, —C₍₁₋₂₅₎alkenyl, —C(O)C₍₁₋₂₅₎alkyl, or —C(O)C₍₁₋₂₅₎alkenyl, wherein the —C₍₁₋₂₅₎alkyl, —C₍₁₋₂₅₎alkenyl, —C(O)C₍₁₋₂₅₎alkyl, and —C(O)C₍₁₋₂₅₎alkenyl are optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl;
provided that no more than two of R^{G1}, R^{G2} and R^{G3} are H.

In some embodiments, the glyceride or acylglycol of Formula I has a structure according to Formula Ia or Ib:

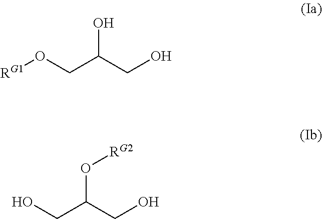

wherein R^{G1} and R^{G2} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In some embodiments, the glyceride or acylglycol of Formula I has a structure according to Formula Ia:

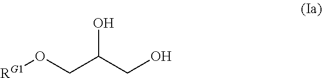

wherein R^{G1} is-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In some embodiments of the glyceride or acylglycol of Formula Ia, R^{G1} is-C₍₁₋₂₅₎alkyl, —C₍₁₋₂₅₎alkenyl, —C(O)C₍₁₋₂₅₎alkyl, or —C(O)C₍₁₋₂₅₎alkenyl.

In some embodiments of the glyceride or acylglycol of Formula Ia, R^{G1} is-C₍₁₁₋₂₅₎alkyl, —C₍₁₁₋₂₅₎alkenyl, —C(O)C₍₁₁₋₂₅₎alkyl, or —C(O)C₍₁₁₋₂₅₎alkenyl.

In some embodiments, the glyceride or acylglycol of Formula I has a structure according to Formula Ib:

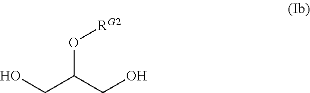

wherein R^{G2} is-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In some embodiments of the glyceride or acylglycol of Formula Ib, R^{G2} is-C₍₁₋₂₅₎alkyl, —C₍₁₋₂₅₎alkenyl, —C(O)C₍₁₋₂₅₎alkyl, or —C(O)C₍₁₋₂₅₎alkenyl.

In some embodiments of the glyceride or acylglycol of Formula Ib, R^{G2} is-C₍₁₁₋₂₅₎alkyl, —C₍₁₁₋₂₅₎alkenyl, —C(O)C₍₁₁₋₂₅₎alkyl, or —C(O)C₍₁₁₋₂₅₎alkenyl.

In some embodiments of the glyceride or acylglycol of Formula I, Ia, or Ib, R^{G1} and R^{G2} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl.

In some embodiments of the glyceride or acylglycol of Formula I, Ia, or Ib, R^{G1} and R^{G2} are each independently-C₍₁₁₋₂₅₎alkyl, —C₍₁₁₋₂₅₎alkenyl, —C(O)C₍₁₁₋₂₅₎alkyl, or —C(O)C₍₁₁₋₂₅₎alkenyl.

In some embodiments, the glyceride or acylglycol of Formula I has a structure according to Formula Ic or Id:

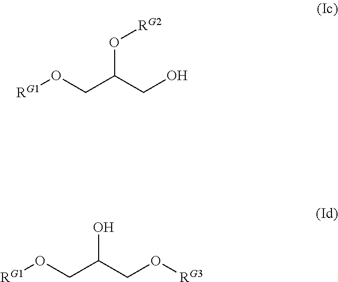

wherein R^{G1}, R^{G2}, and R^{G3} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In some embodiments, the glyceride or acylglycol of Formula I has a structure according to Formula Ic:

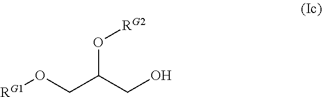

wherein R^{G1} and R^{G2} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In some embodiments of the glyceride or acylglycol of Formula Ic, R^{G1} and R^{G2} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl.

In some embodiments of the glyceride or acylglycol of Formula Ic, R^{G1} and R^{G2} are each independently —C(O)C₍₁₋₂₅₎alkyl or —C(O)C₍₁₋₂₅₎alkenyl.

In some embodiments of the glyceride or acylglycol of Formula Ic, R^{G1} is —C(O)C₍₃₋₂₅₎alkyl or —C(O)C₍₃₋₂₅₎alkenyl.

In some embodiments of the glyceride or acylglycol of Formula Ic, R^{G2} is —C(O)C₍₁₋₂₅₎alkyl or —C(O)C₍₁₋₂₅₎alkenyl.

In some embodiments, the glyceride or acylglycol of Formula I has a structure according to Formula Id:

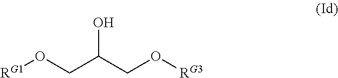

wherein R^{G1} and R^{G3} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In some embodiments of the glyceride or acylglycol of Formula Id, R^{G1} and R^{G3} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl.

In some embodiments of the glyceride or acylglycol of Formula I, Ic, or Id, R^{G1}, R^{G2}, and R^{G3} are each independently —C(O)C₍₁₋₂₅₎alkyl or —C(O)C₍₁₋₂₅₎alkenyl.

In some embodiments of the glyceride or acylglycol of Formula I, Ic, or Id, R^{G1} is —C(O)C₍₃₋₂₅₎alkyl or —C(O)C₍₃₋₂₅₎alkenyl.

In some embodiments of the glyceride or acylglycol of Formula I, Ic, or Id, R^{G2} is —C(O)C₍₁₋₂₅₎alkyl or —C(O)C₍₁₋₂₅₎alkenyl.

In some embodiments of the glyceride or acylglycol of Formula I, Ic, or Id, R^{G3} is —C(O)C₍₃₋₂₅₎alkyl or —C(O)C₍₃₋₂₅₎alkenyl.

In some embodiments of the glyceride or acylglycol of Formula I, Ic, or Id:
R^{G1} is —C(O)C₍₃₋₂₅₎alkyl, or —C(O)C₍₃₋₂₅₎alkenyl;
R^{G2} is —C(O)C₍₁₋₂₅₎alkyl, or —C(O)C₍₁₋₂₅₎alkenyl; and
R^{G3} is —C(O)C₍₃₋₂₅₎alkyl, or —C(O)C₍₃₋₂₅₎alkenyl.

In some embodiments, the glyceride or acylglycol of Formula I has a structure according to Formula Ie:

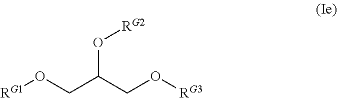

wherein R^{G1}, R^{G2}, and R^{G3} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In some embodiments, the glyceride or acylglycol of Formula I has a structure according to Formula Ie, wherein R^{G1}, R^{G2}, and R^{G3} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl.

In some embodiments of the glyceride or acylglycol of Formula I or Ie, R^{G1} is —C(O)C₍₁₋₂₅₎alkyl or —C(O)C₍₁₋₂₅₎alkenyl.

In some embodiments of the glyceride or acylglycol of Formula I or Ie, R^{G2} is —C(O)C₍₁₋₂₅₎alkyl or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In some embodiments of the glyceride or acylglycol of Formula I or Ie, R^{G3} is —C(O)C₍₁₋₂₅₎alkyl or —C(O)C₍₁₋₂₅₎alkenyl.

In some embodiments of the glyceride or acylglycol of Formula I or Ie:
R^{G1} is —C(O)C₍₁₋₂₅₎alkyl or —C(O)C₍₁₋₂₅₎alkenyl;
R^{G2} is —C(O)C₍₁₋₂₅₎alkyl or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl; and
R^{G3} is —C(O)C₍₁₋₂₅₎alkyl or —C(O)C₍₁₋₂₅₎alkenyl.

In some embodiments of the glyceride or acylglycol of Formula I or Ie, R^{G1}, R^{G2}, and R^{G3} are each independently —C(O)C₍₇₋₂₁₎alkyl or —C(O)C₍₇₋₂₁₎alkenyl.

In some embodiments, the glyceride or acylglycol has a structure according to Formula II:

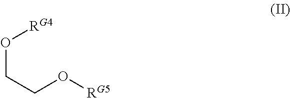

wherein:
R^{G4} is-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl;
R^{G5} is H, —C₍₁₋₂₅₎alkyl, —C₍₁₋₂₅₎alkenyl, —C(O)C₍₁₋₂₅₎alkyl, or —C(O)C₍₁₋₂₅₎alkenyl, wherein the —C₍₁₋₂₅₎alkyl, —C₍₁₋₂₅₎alkenyl, —C(O)C₍₁₋₂₅₎alkyl, and —C(O)C₍₁₋₂₅₎alkenyl are optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In some embodiments, the glyceride or acylglycol of Formula II has a structure according to Formula IIa or IIb:

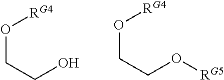

wherein R^{G4} and R^{G5} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In some embodiments, the glyceride or acylglycol of Formula II has a structure according to Formula IIa:

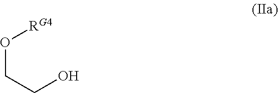

wherein R^{G4} is-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In some embodiments, the glyceride or acylglycol of Formula II has a structure according to Formula IIa, wherein R^{G4} is-C₍₁₋₂₅₎alkyl, —C₍₁₋₂₅₎alkenyl, —C(O)C₍₁₋₂₅₎alkyl, or —C(O)C₍₁₋₂₅₎alkenyl.

In some embodiments, the glyceride or acylglycol of Formula II has a structure according to Formula IIb:

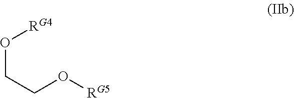

wherein R^{G4} and R^{G5} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In some embodiments, the glyceride or acylglycol of Formula II has a structure according to Formula IIb, wherein R^{G4} and R^{G5} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl.

In one or more embodiment of the glyceride or acylglycol of Formula II, IIa, or IIb, R^{G4} and R^{G5} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl.

In one or more embodiments of the glyceride or acylglycol of Formula II, IIa, or IIIb, R^{G4} and R^{G5} are each independently —C(O)C₍₁₋₂₅₎alkyl or —C(O)C₍₁₋₂₅₎alkenyl.

In one or more embodiments, the glyceride or acylglycol is selected from the group consisting of:

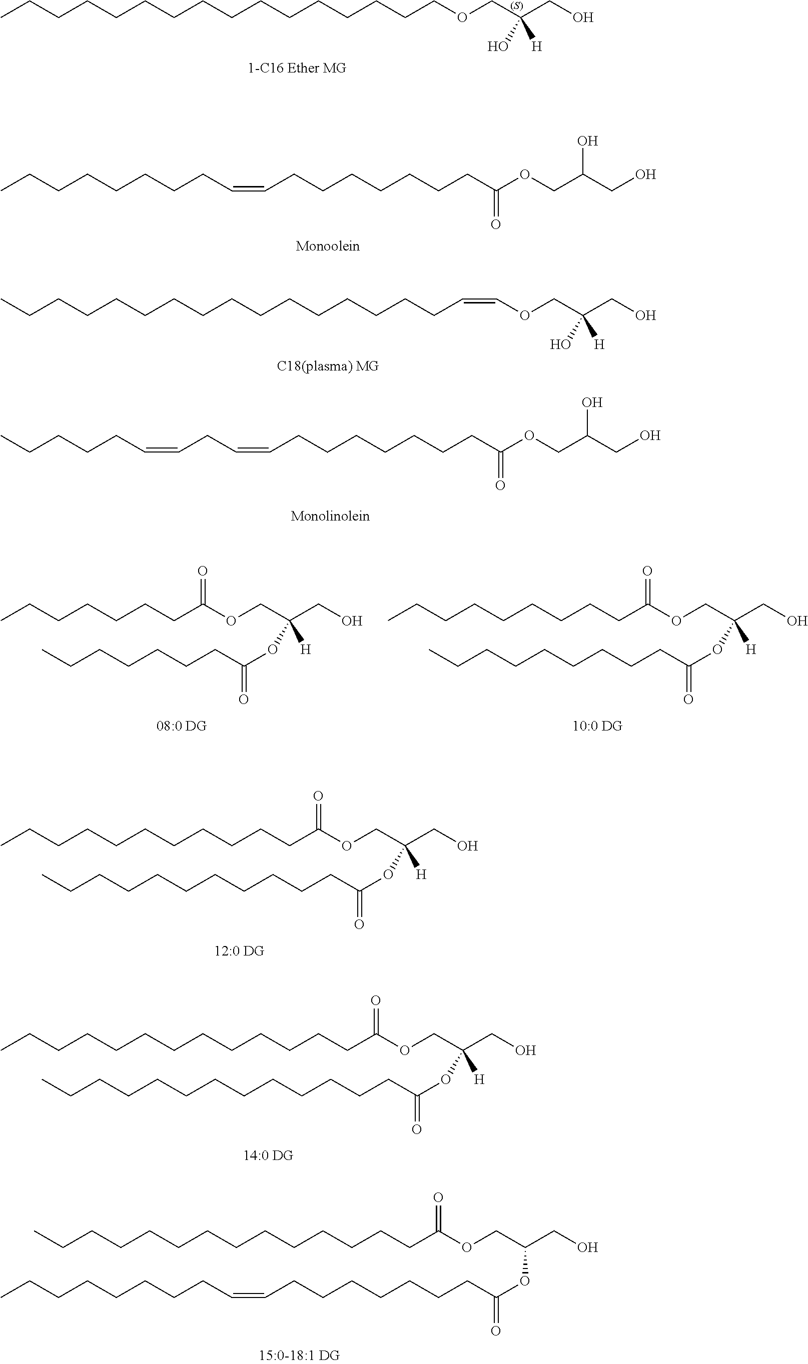

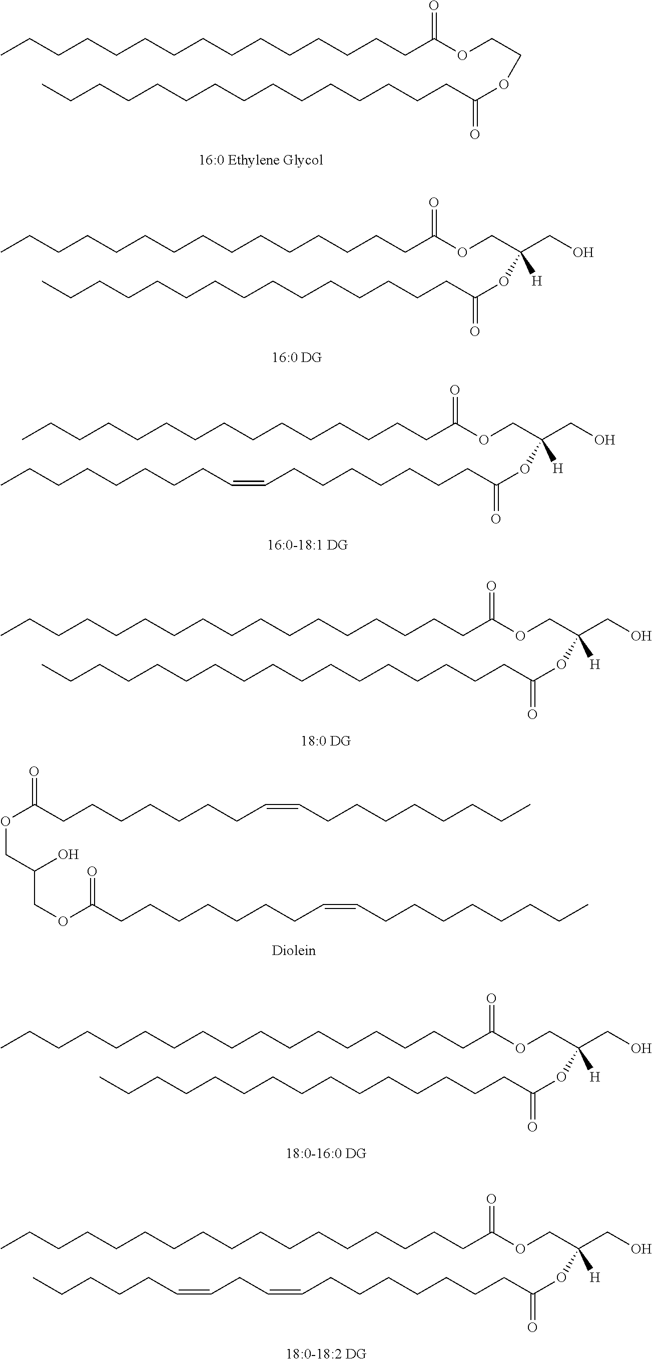

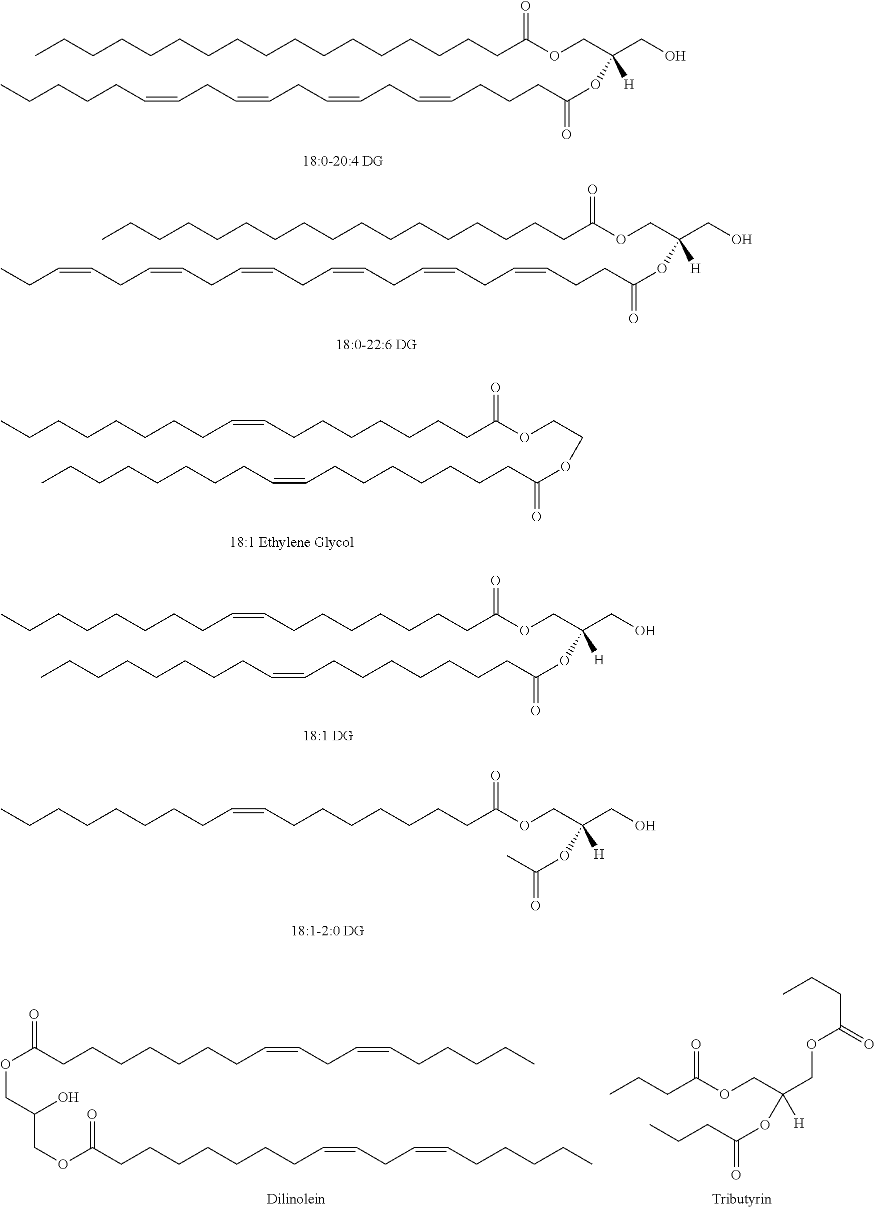

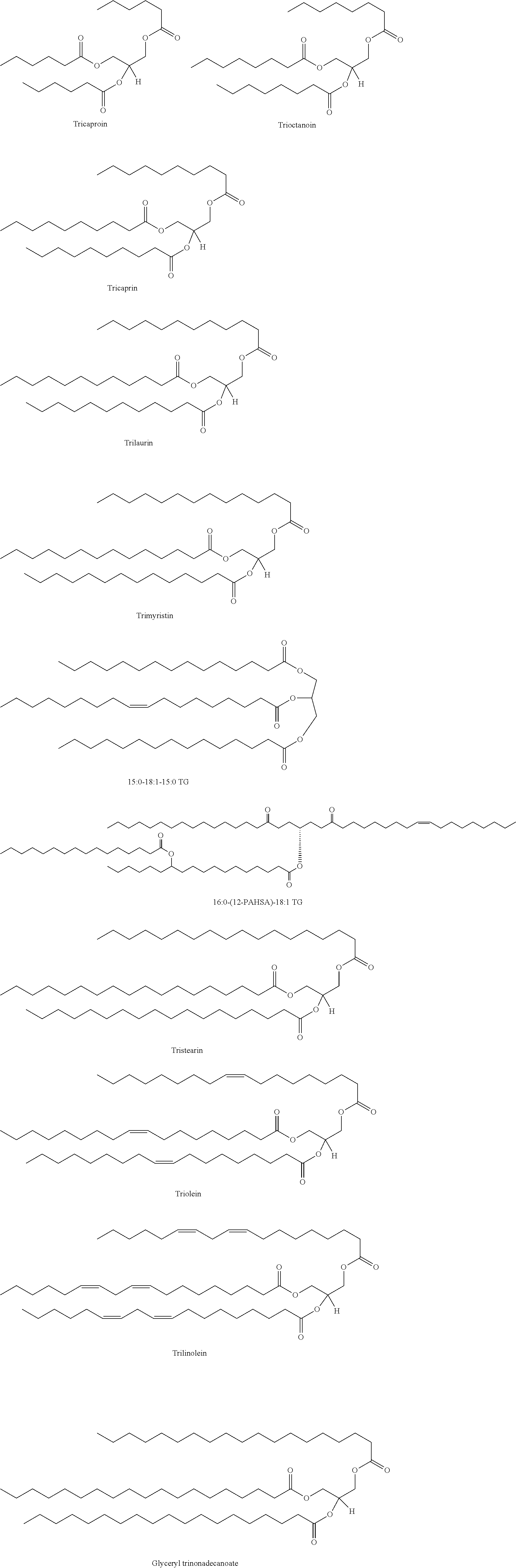

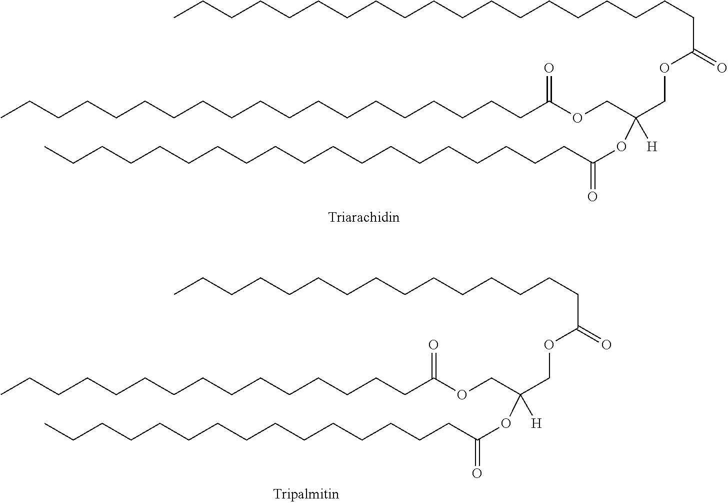 [SMILES: CCCCCCCCCCCCCCCCCCCC(=O)OCC(COC(=O)CCCCCCCCCCCCCCCCCCC)OC(=O)CCCCCCCCCCCCCCCCCCC ; CCCCCCCCCCCCCCCC(=O)OCC(COC(=O)CCCCCCCCCCCCCCC)OC(=O)CCCCCCCCCCCCCCC]

or a combination thereof.

In some embodiments, the glyceride or acylglycol is C18(plasm) MG. In some embodiments, the glyceride or acylglycol is 08:0 DG. In some embodiments, the glyceride or acylglycol is 10:0 DG. In some embodiments, the glyceride or acylglycol is 12:0 DG. In some embodiments, the glyceride or acylglycol is 14:0 DG. In some embodiments, the glyceride or acylglycol is 15:0-18:1 DG. In some embodiments, the glyceride or acylglycol is 16:0 ethylene glycol. In some embodiments, the glyceride or acylglycol is 16:0 DG. In some embodiments, the glyceride or acylglycol is 16:0-18:1 DG. In some embodiments, the glyceride or acylglycol is 18:0 DG.

In some embodiments, the glyceride or acylglycol is 18:0-16:0 DG. In some embodiments, the glyceride or acylglycol is 18:0-18:2 DG. In some embodiments, the glyceride or acylglycol is 18:0-20:4 DG. In some embodiments, the glyceride or acylglycol is 18:0-22:6 DG. In some embodiments, the glyceride or acylglycol is 18:1 ethylene glycol. In some embodiments, the glyceride or acylglycol is 18:1 DG. In some embodiments, the glyceride or acylglycol is tributyrin. In some embodiments, the glyceride or acylglycol is tricaproin. In some embodiments, the glyceride or acylglycol is trioctanoin. In some embodiments, the glyceride or acylglycol is 15:0-18:1-15:0 TG. In some embodiments, the glyceride or acylglycol is 16:0-(12-PAHSA)-18:1 TG.

In some embodiments, the glyceride or acylglycol is selected from the group consisting of:

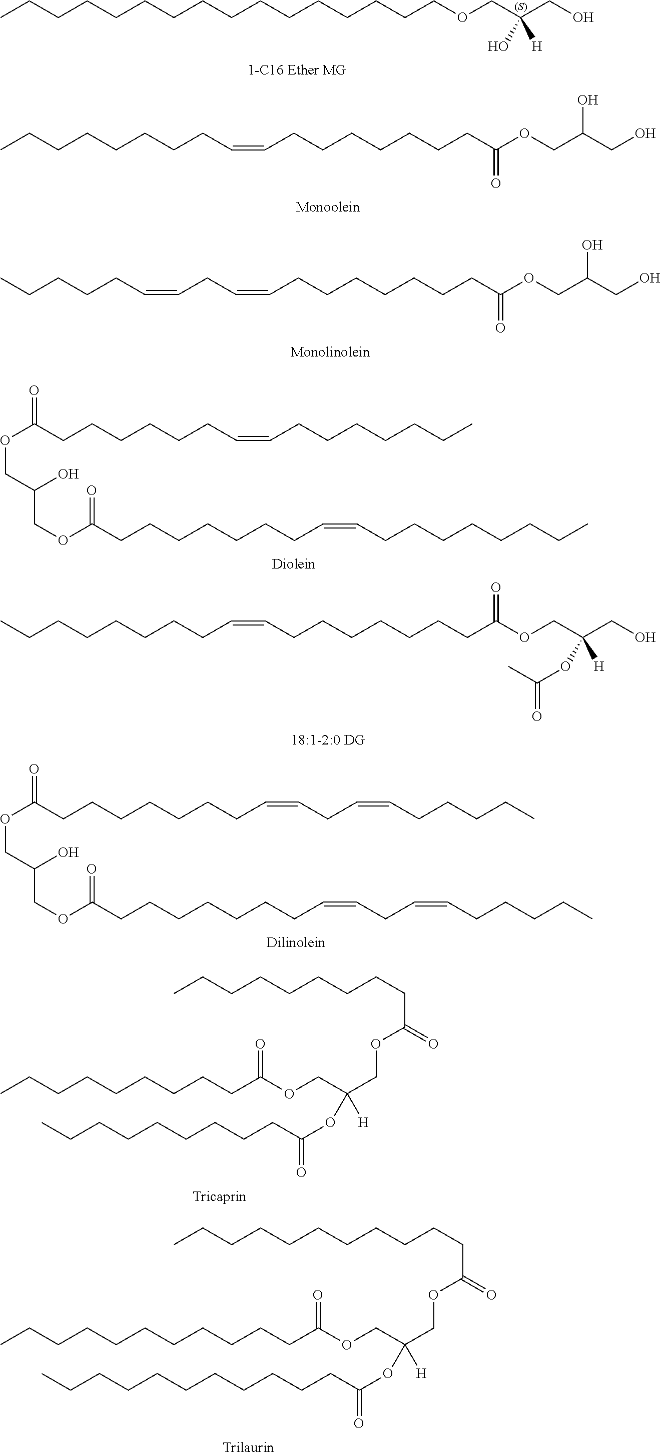

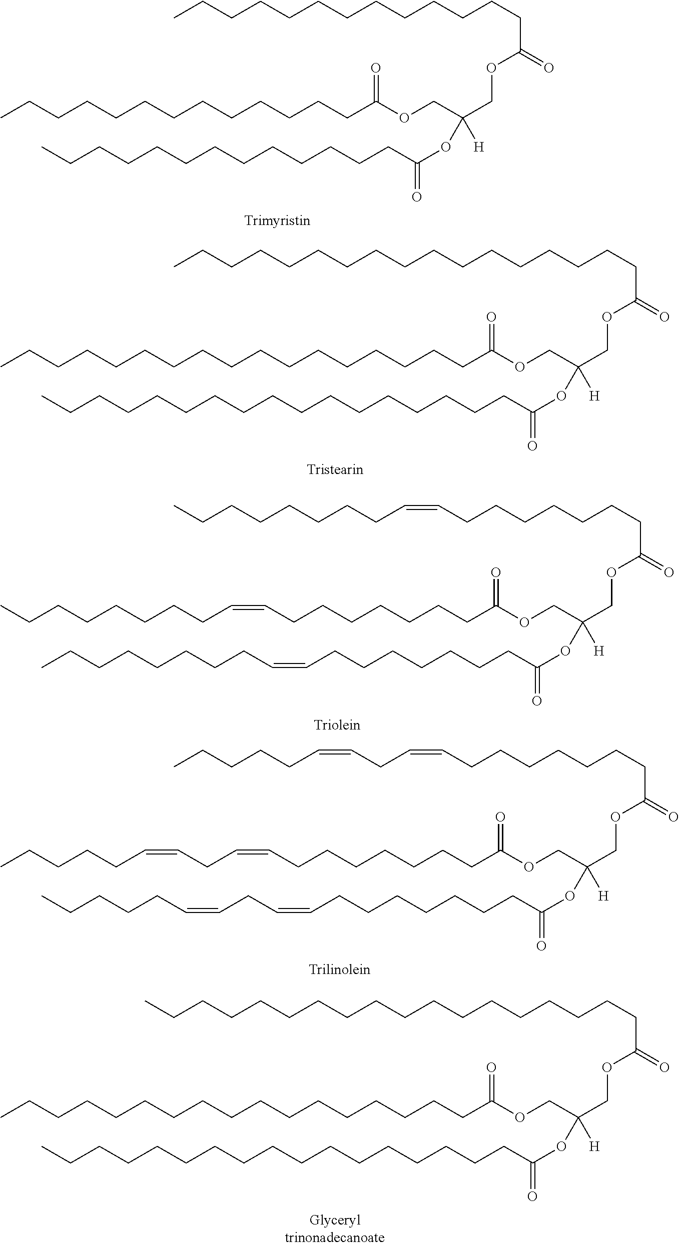 [SMILES: CCCCCCCCCCCCCC(=O)OCC(COC(=O)CCCCCCCCCCCCC)OC(=O)CCCCCCCCCCCCC ; CCCCCCCCCCCCCCCCCC(=O)OCC(COC(=O)CCCCCCCCCCCCCCCCC)OC(=O)CCCCCCCCCCCCCCCCC ; CCCCCCCC/C=C\CCCCCCCC(=O)OCC(COC(=O)CCCCCCC/C=C\CCCCCCCC)OC(=O)CCCCCCC/C=C\CCCCCCCC ; CCCCC/C=C\C/C=C\CCCCCCCC(=O)OCC(COC(=O)CCCCCCC/C=C\C/C=C\CCCCC)OC(=O)CCCCCCC/C=C\C/C=C\CCCCC ; CCCCCCCCCCCCCCCCCCC(=O)OCC(COC(=O)CCCCCCCCCCCCCCCCC)OC(=O)CCCCCCCCCCCCCCCCC]

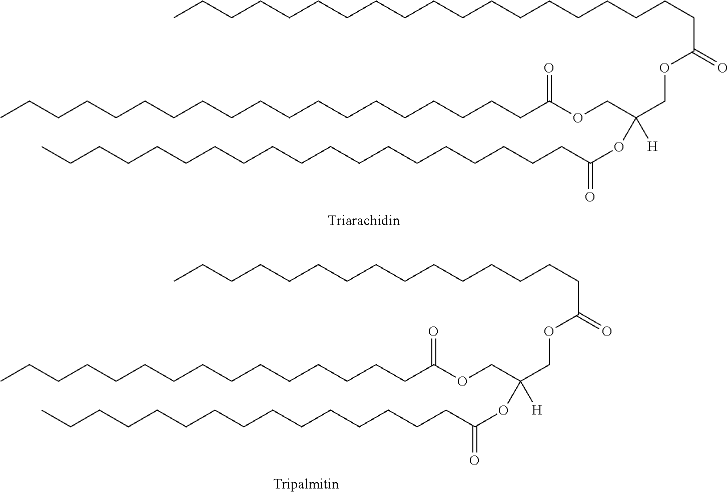 [SMILES: CCCCCCCCCCCCCCCCCCCC(=O)OCC(COC(=O)CCCCCCCCCCCCCCCCCCC)OC(=O)CCCCCCCCCCCCCCCCCCC ; CCCCCCCCCCCCCCCC(=O)OCC(COC(=O)CCCCCCCCCCCCCCC)OC(=O)CCCCCCCCCCCCCCC]

or a combination thereof.

In some embodiments, the glyceride or acylglycol is 1-C16 ether MG. In some embodiments, the glyceride or acylglycol is 18:1-2:0 DG. In some embodiments, the glyceride or acylglycol is trilaurin. In some embodiments, the glyceride or acylglycol is trilinolein. In some embodiments, the glyceride or acylglycol is glyceryl trinonadeanoate. In some embodiments, the glyceride or acylglycol is tripalmitin.

In some embodiments, the glyceride or acylglycol is selected from the group consisting of:

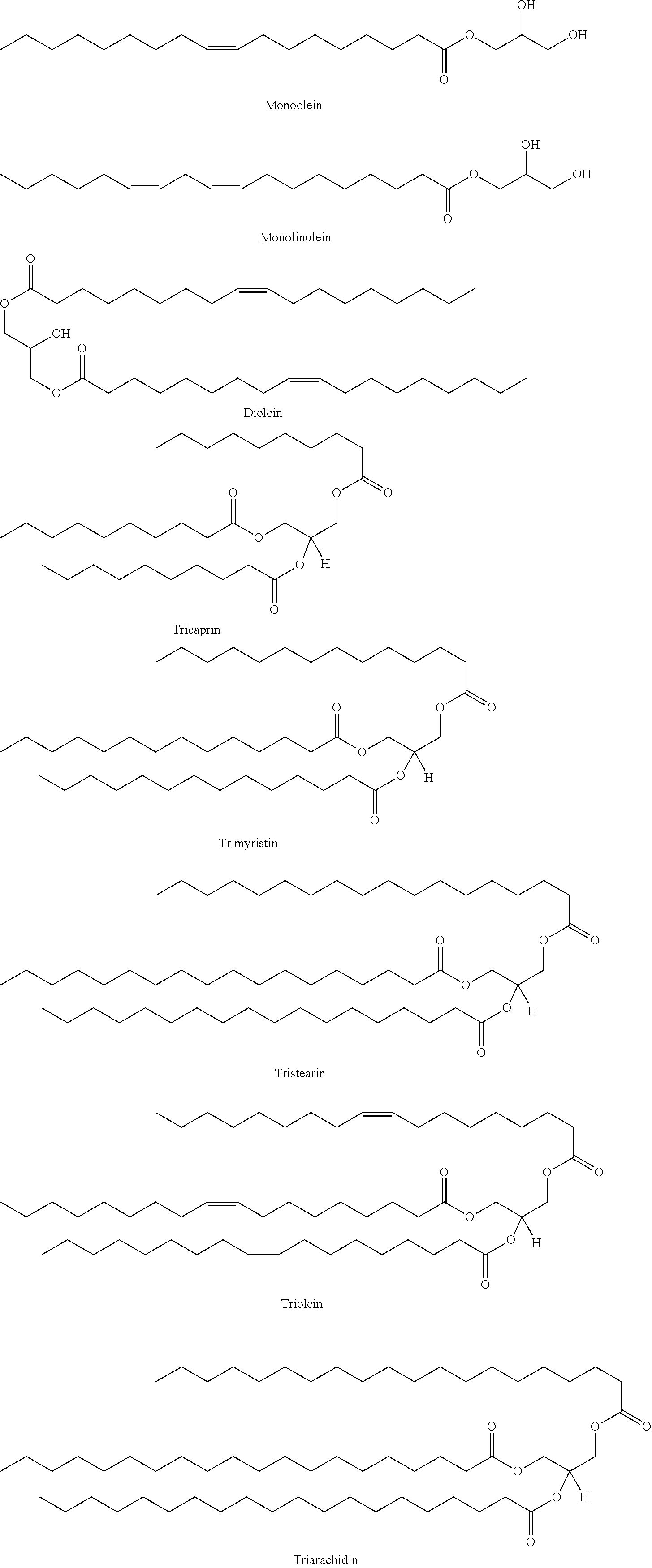

or a combination thereof.

In some embodiments, the glyceride or acylgycol is monoolein. In some embodiments, the glyceride or acylglycol is monolinolein. In some embodiments, the glyceride or acylglycol is trimyristin. In some embodiments, the glyceride or acylglycol is tristearin. In some embodiments, the glyceride or acylglycol is triarchidin.

In some embodiments, the glyceride or acylglycol is selected from:

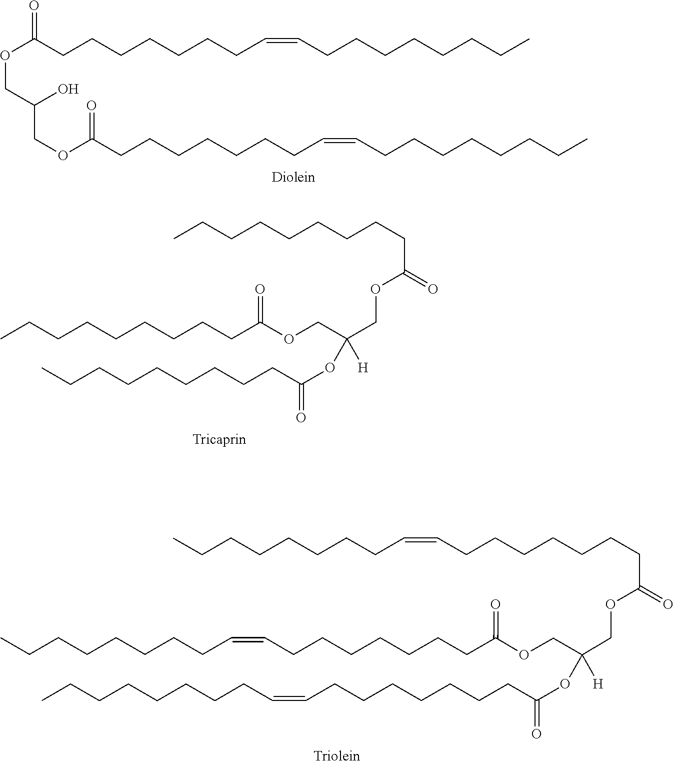

In some embodiments, the glyceride or acylglycol is diolein. In some embodiments, the glyceride or acylglycol is tricaprin. In some embodiments, the glyceride or acylglycol is triolein.

In one or more embodiments, the glyceride or acylglycol is hydrolyzable by lipase.

### Structural Lipids

The structural lipid component provides stability to the lipid bilayer structure within the nanoparticle. In some embodiments, the LNPs comprise one or more structural lipids. Suitable cholesterol-based lipids include, for example: DC-Choi (N,N-dimethyl-N-ethylcarboxamidocholesterol), 1,4-bis(3-N-oleylamino-propyl)piperazine (Gao et al., Biochem Biophys Res Comm. (1991) 179:280; Wolf et al., BioTechniques (1997) 23:139; U.S. Pat. No. 5,744,335), imidazole cholesterol ester (“ICE”; WO2011/068810), sitosterol (22,23-dihydrostigmasterol), β-sitosterol, sitostanol, fucosterol, stigmasterol (stigmasta-5,22-dien-3-ol), ergosterol; desmosterol (3ß-hydroxy-5,24-cholestadiene); lanosterol (8,24-lanostadien-3b-ol); 7-dehydrocholesterol (Δ5,7-cholesterol); dihydrolanosterol (24,25-dihydrolanosterol); zymosterol (5α-cholesta-8,24-dien-3ß-ol); lathosterol (5α-cholest-7-en-3ß-ol); diosgenin ((3β,25R)-spirost-5-en-3-ol); campesterol (campest-5-en-3ß-ol); campestanol (5a-campestan-3b-ol); 24-methylene cholesterol (5,24(28)-cholestadien-24-methylen-3ß-ol); cholesteryl margarate (cholest-5-en-3ß-yl heptadecanoate); cholesteryl oleate; cholesteryl stearate and other modified forms of cholesterol.

In some embodiments, the structural lipid is cholesterol.

### Stealth Lipids

The stealth lipid component provides control over particle size and stability of the nanoparticle. The addition of such components may prevent complex aggregation and provide a means for increasing circulation lifetime and increasing the delivery of the lipid-nucleic acid pharmaceutical composition to target tissues.

In some embodiments, the stealth lipid is a polyethylene glycol-conjugated (PEGylated) lipid. These components may be selected to rapidly exchange out of the pharmaceutical composition in vivo (see, e.g., U.S. Pat. No. 5,885,613).

Contemplated PEGylated lipids include, but are not limited to, a polyethylene glycol (PEG) chain of up to 5 kDa in length covalently attached to a lipid with alkyl chain(s) of C₆-C₂₀ (e.g., C₈, C₁₀, C₁₂, C₁₄, C₁₆, or C₁₈) length, such as a derivatized ceramide (e.g., N-octanoyl-sphingosine-1-[succinyl(methoxypolyethylene glycol)](C8 PEG ceramide)). In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); or 1,2-distearoyl-rac-glycero-polyethelene glycol (DSG-PEG).

In some embodiments, the PEG has a high molecular weight, e.g., 2000-2400 g/mol. In some embodiments, the PEG is PEG2000 (or PEG-2K). In some embodiments, the PEGylated lipid herein is DMG-PEG2000, DSPE-PEG2000, DLPE-PEG2000, DSG-PEG2000, or C8 PEG2000. In some embodiments, the PEGylated lipid is dimyristoyl-PEG2000 (DMG-PEG2000).

### Helper Lipids

A helper lipid enhances the structural stability of the LNP and helps the LNP in endosome escape. It improves uptake and release of the mRNA drug payload. In some embodiments, the helper lipid is a zwitterionic lipid. Without wishing to be bound by theory, the helper lipid can have fusogenic properties for enhancing uptake and release of the drug payload. Examples of helper lipids are 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), 1,2-dilauroyl-sn-glycero-3-phosphocholine (DLPC), 1,2-Distearoylphosphatidylethanolamine (DSPE), and 1,2-dilauroyl-sn-glycero-3-phosphoethanolamine (DLPE).

Other exemplary helper lipids are dioleoylphosphatidylcholine (DOPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a combination thereof.

In particular embodiments, the helper lipid is DOPE.

### Combinations of Lipid Components and Molar Ratios

In some embodiments, the LNP comprises (I) a cationic lipid; (II) a glyceride or an acylglycol selected from the group consisting of 1-C16 ether MG, monoolein, C18(plasm) MG, monolinolein, 08:0 DG, 10:0 DG, 12:0 DG, 14:0 DG, 15:0-18:1 DG, 16:0 ethylene glycol, 16:0 DG, 16:0-18:1 DG, 18:0 DG, diolein, 18:0-16:0 DG, 18:0-18:2 DG, 18:0-20:4 DG, 18:0-22:6 DG, 18:1 ethylene glycol, 18:1 DG, 18:1-2:0 DG, dilinolein, tributyrin, tricaproin, trioctanoin, tricaprin, trilaurin, trimyristin, 15:0-18:1-15:0 TG, 16:0-(12-PAHSA)-18:1 TG, tristearin, triolein, trilinolein, glyceryl trinonadecanoate, triarachidin, tripalmitin and a combination thereof; and (III) a structural lipid.

In some embodiments, the LNP comprises (I) a cationic lipid; (II) a glyceride or an acylglycol selected from the group consisting of 1-C16 ether MG, monoolein, C18(plasm) MG, monolinolein, 08:0 DG, 10:0 DG, 12:0 DG, 14:0 DG, 15:0-18:1 DG, 16:0 ethylene glycol, 16:0 DG, 16:0-18:1 DG, 18:0 DG, diolein, 18:0-16:0 DG, 18:0-18:2 DG, 18:0-20:4 DG, 18:0-22:6 DG, 18:1 ethylene glycol, 18:1 DG, 18:1-2:0 DG, dilinolein, tributyrin, tricaproin, trioctanoin, tricaprin, trilaurin, trimyristin, 15:0-18:1-15:0 TG, 16:0-(12-PAHSA)-18:1 TG, tristearin, triolein, trilinolein, glyceryl trinonadecanoate, triarachidin, tripalmitin and a combination thereof; and (III) a helper lipid.

In some embodiments, the LNP comprises (I) a cationic lipid; (II) a glyceride or an acylglycol selected from the group consisting of 1-C16 ether MG, monoolein, C18(plasm) MG, monolinolein, 08:0 DG, 10:0 DG, 12:0 DG, 14:0 DG, 15:0-18:1 DG, 16:0 ethylene glycol, 16:0 DG, 16:0-18:1 DG, 18:0 DG, diolein, 18:0-16:0 DG, 18:0-18:2 DG, 18:0-20:4 DG, 18:0-22:6 DG, 18:1 ethylene glycol, 18:1 DG, 18:1-2:0 DG, dilinolein, tributyrin, tricaproin, trioctanoin, tricaprin, trilaurin, trimyristin, 15:0-18:1-15:0 TG, 16:0-(12-PAHSA)-18:1 TG, tristearin, triolein, trilinolein, glyceryl trinonadecanoate, triarachidin, tripalmitin and a combination thereof; and (III) a stealth lipid.

In some embodiments, the LNP comprises (I) a cationic lipid; (II) a glyceride or an acylglycol selected from the group consisting of 1-C16 ether MG, monoolein, C18(plasm) MG, monolinolein, 08:0 DG, 10:0 DG, 12:0 DG, 14:0 DG, 15:0-18:1 DG, 16:0 ethylene glycol, 16:0 DG, 16:0-18:1 DG, 18:0 DG, diolein, 18:0-16:0 DG, 18:0-18:2 DG, 18:0-20:4 DG, 18:0-22:6 DG, 18:1 ethylene glycol, 18:1 DG, 18:1-2:0 DG, dilinolein, tributyrin, tricaproin, trioctanoin, tricaprin, trilaurin, trimyristin, 15:0-18:1-15:0 TG, 16:0-(12-PAHSA)-18:1 TG, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid; (II) a glyceride or an acylglycol selected from the group consisting of monoolein, monolinolein, diolein, 18:1-2:0 DG, dilinolein, tributyrin, tricaproin, trioctanoin, tricaprin, trilaurin, trimyristin, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid; (II) a glyceride or an acylglycol selected from the group consisting of monoolein, monolinolein, diolein, 18:1-2:0 DG, dilinolein, tricaprin, trilaurin, trimyristin, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid; (II) a glyceride or an acylglycol selected from the group consisting of monoolein, monolinolein, diolein, tricaprin, trimyristin, tristearin, triolein, triarachidin, and a combination thereof; (III) a structural lipid; (IV) a helper lipid; and (V) a stealth lipid.

In some embodiments, the LNP comprises (I) a cationic lipid; (II) a glyceride or an acylglycol selected from the group consisting of diolein, tricaprin, triolein, and a combination thereof; (III) a structural lipid; (IV) a helper lipid; and (V) a stealth lipid.

In some embodiments, the LNP comprises (I) a cationic lipid having a structure according to Formula CAT-I or Formula CAT-II; (II) a glyceride or an acylglycol; and (III) a structural lipid.

In some embodiments, the LNP comprises (I) a cationic lipid having a structure according to Formula CAT-I or Formula CAT-II; (II) a glyceride or an acylglycol; and (III) a helper lipid.

In some embodiments, the LNP comprises (I) a cationic lipid having a structure according to Formula CAT-I or Formula CAT-II; (II) a glyceride or an acylglycol; and (III) a stealth lipid.

In some embodiments, the LNP comprises (I) a cationic lipid having a structure according to Formula CAT-I or Formula CAT-II; (II) a glyceride or an acylglycol; (III) a structural lipid; (IV) a helper lipid; and (V) a stealth lipid.

In some embodiments, the LNP comprises (I) a cationic lipid having a structure according to Formula CAT-I; (II) a glyceride or an acylglycol; (III) a structural lipid; (IV) a helper lipid; and (V) a stealth lipid.

In some embodiments, the LNP comprises (I) a cationic lipid having a structure according to Formula CAT-II; (II) a glyceride or an acylglycol; (III) a structural lipid; (IV) a helper lipid; and (V) a stealth lipid.

In some embodiments, the LNP comprises (I) a cationic lipid that is cKK-E10, OF-02, or GL-HEPES-E3-E12-DS-4-E10; (II) a glyceride or an acylglycol; (III) a structural lipid; (IV) a helper lipid; and (V) a stealth lipid.

In some embodiments, the LNP comprises (I) a cationic lipid that is TL1-12D-DMA, HEP-E4-E12, BAL-020, or BAL-005; (II) a glyceride or an acylglycol; (III) a structural lipid; (IV) a helper lipid; and (V) a stealth lipid.

In some embodiments, the LNP comprises (I) a cationic lipid having a structure according to Formula CAT-I or Formula CAT-II; (II) a glyceride or an acylglycol having a structure according to Formula I or Formula II; and (III) a structural lipid.

In some embodiments, the LNP comprises (I) a cationic lipid having a structure according to Formula CAT-I or Formula CAT-II; (II) a glyceride or an acylglycol having a structure according to Formula I or Formula II; and (III) a helper lipid.

In some embodiments, the LNP comprises (I) a cationic lipid having a structure according to Formula CAT-I or Formula CAT-II; (II) a glyceride or an acylglycol having a structure according to Formula I or Formula II; and (III) a stealth lipid.

In some embodiments, the LNP comprises (I) a cationic lipid having a structure according to Formula CAT-I or Formula CAT-II; (II) a glyceride or an acylglycol having a structure according to Formula I or Formula II; (III) a structural lipid; (IV) a helper lipid; and (V) a stealth lipid.

In some embodiments, the LNP comprises (I) a cationic lipid having a structure according to Formula CAT-I; (II) a glyceride or an acylglycol having a structure according to Formula I or Formula II; (III) a structural lipid; (IV) a helper lipid; and (V) a stealth lipid.

In some embodiments, the LNP comprises (I) a cationic lipid having a structure according to Formula CAT-II; (II) a glyceride or an acylglycol having a structure according to Formula I or Formula II; (III) a structural lipid; (IV) a helper lipid; and (V) a stealth lipid.

In some embodiments, the LNP comprises (I) a cationic lipid having a structure according to Formula CAT-I or Formula CAT-II; (II) a glyceride or an acylglycol having a structure according to Formula I; (III) a structural lipid; (IV) a helper lipid; and (V) a stealth lipid.

In some embodiments, the LNP comprises (I) a cationic lipid having a structure according to Formula CAT-I or Formula CAT-II; (II) a glyceride or an acylglycol having a structure according to Formula Ia or Formula Ib; (III) a structural lipid; (IV) a helper lipid; and (V) a stealth lipid.

In some embodiments, the LNP comprises (I) a cationic lipid having a structure according to Formula CAT-I or Formula CAT-II; (II) a glyceride or an acylglycol having a structure according to Formula Ic or Formula Id; (III) a structural lipid; (IV) a helper lipid; and (V) a stealth lipid.

In some embodiments, the LNP comprises (I) a cationic lipid having a structure according to Formula CAT-I or Formula CAT-II; (II) a glyceride or an acylglycol having a structure according to Formula Ie; (III) a structural lipid; (IV) a helper lipid; and (V) a stealth lipid.

In some embodiments, the LNP comprises (I) a cationic lipid having a structure according to Formula CAT-I or Formula CAT-II; (II) a glyceride or an acylglycol having a structure according to Formula I, Formula Ia, Formula Ib, Formula Ic, Formula Id, or Formula Ie; (III) a structural lipid; (IV) a helper lipid; and (V) a stealth lipid.

In some embodiments, the LNP comprises (I) a cationic lipid having a structure according to Formula CAT-I; (II) a glyceride or an acylglycol having a structure according to Formula I, Formula Ia, Formula Ib, Formula Ic, Formula Id, or Formula Ie; (III) a structural lipid; (IV) a helper lipid; and (V) a stealth lipid.

In some embodiments, the LNP comprises (I) a cationic lipid having a structure according to Formula CAT-II; (II) a glyceride or an acylglycol having a structure according to Formula I, Formula Ia, Formula Ib, Formula Ic, Formula Id, or Formula Ie; (III) a structural lipid; (IV) a helper lipid; and (V) a stealth lipid.

In some embodiments, the LNP comprises (I) a cationic lipid having a structure according to Formula CAT-I or Formula CAT-II; (II) a glyceride or an acylglycol having a structure according to Formula II; (III) a structural lipid; (IV) a helper lipid; and (V) a stealth lipid.

In some embodiments, the LNP comprises (I) a cationic lipid having a structure according to Formula CAT-I or Formula CAT-II; (II) a glyceride or an acylglycol having a structure according to Formula Ila or Formula IIb; (III) a structural lipid; (IV) a helper lipid; and (V) a stealth lipid.

In some embodiments, the LNP comprises (I) a cationic lipid having a structure according to Formula CAT-I; (II) a glyceride or an acylglycol having a structure according to Formula Ila or Formula IIb; (III) a structural lipid; (IV) a helper lipid; and (V) a stealth lipid.

In some embodiments, the LNP comprises (I) a cationic lipid having a structure according to Formula CAT-II; (II) a glyceride or an acylglycol having a structure according to Formula IIa or Formula IIb; (III) a structural lipid; (IV) a helper lipid; and (V) a stealth lipid.

In some embodiments, the LNP comprises (I) a cationic lipid having a structure according to Formula CAT-I or Formula CAT-II; (II) a glyceride or an acylglycol having a structure according to Formula I, Formula Ia, Formula Ib, Formula Ic, Formula Id, Formula Ie, Formula II, Formula IIa, or Formula IIb; (III) a structural lipid; (IV) a helper lipid; and (V) a stealth lipid.

In some embodiments, the LNP comprises (I) a cationic lipid having a structure according to Formula CAT-I; (II) a glyceride or an acylglycol having a structure according to Formula I, Formula Ia, Formula Ib, Formula Ic, Formula Id, Formula Ie, Formula II, Formula IIa, or Formula IIb; (III) a structural lipid; (IV) a helper lipid; and (V) a stealth lipid.

In some embodiments, the LNP comprises (I) a cationic lipid having a structure according to Formula CAT-II; (II) a glyceride or an acylglycol having a structure according to Formula I, Formula Ia, Formula Ib, Formula Ic, Formula Id, Formula IIe, Formula II, Formula IIa, or Formula IIb; (III) a structural lipid; (IV) a helper lipid; and (V) a stealth lipid.

In some embodiments, the LNP comprises (I) a cationic lipid having a structure according to Formula CAT-I or Formula CAT-II; (II) a glyceride or an acylglycol selected from the group consisting of 1-C16 ether MG, monoolein, C18(plasm) MG, monolinolein, 08:0 DG, 10:0 DG, 12:0 DG, 14:0 DG, 15:0-18:1 DG, 16:0 ethylene glycol, 16:0 DG, 16:0-18:1 DG, 18:0 DG, diolein, 18:0-16:0 DG, 18:0-18:2 DG, 18:0-20:4 DG, 18:0-22:6 DG, 18:1 ethylene glycol, 18:1 DG, 18:1-2:0 DG, dilinolein, tributyrin, tricaproin, trioctanoin, tricaprin, trilaurin, trimyristin, 15:0-18:1-15:0 TG, 16:0-(12-PAHSA)-18:1 TG, tristearin, triolein, trilinolein, glyceryl trinonadecanoate, triarachidin, tripalmitin, and a combination thereof; (III) a structural lipid; (IV) a helper lipid; and (V) a stealth lipid.

In some embodiments, the LNP comprises (I) a cationic lipid having a structure according to Formula CAT-I; (II) a glyceride or an acylglycol selected from the group consisting of 1-C16 ether MG, monoolein, C18(plasm) MG, monolinolein, 08:0 DG, 10:0 DG, 12:0 DG, 14:0 DG, 15:0-18:1 DG, 16:0 ethylene glycol, 16:0 DG, 16:0-18:1 DG, 18:0 DG, diolein, 18:0-16:0 DG, 18:0-18:2 DG, 18:0-20:4 DG, 18:0-22:6 DG, 18:1 ethylene glycol, 18:1 DG, 18:1-2:0 DG, dilinolein, tributyrin, tricaproin, trioctanoin, tricaprin, trilaurin, trimyristin, 15:0-18:1-15:0 TG, 16:0-(12-PAHSA)-18:1 TG, tristearin, triolein, trilinolein, glyceryl trinonadecanoate, triarachidin, tripalmitin and a combination thereof, (III) a structural lipid; (IV) a helper lipid; and (V) a stealth lipid.

In some embodiments, the LNP comprises (I) a cationic lipid having a structure according to Formula CAT-II; (II) a glyceride or an acylglycol selected from the group consisting of 1-C16 ether MG, monoolein, C18(plasm) MG, monolinolein, 08:0 DG, 10:0 DG, 12:0 DG, 14:0 DG, 15:0-18:1 DG, 16:0 ethylene glycol, 16:0 DG, 16:0-18:1 DG, 18:0 DG, diolein, 18:0-16:0 DG, 18:0-18:2 DG, 18:0-20:4 DG, 18:0-22:6 DG, 18:1 ethylene glycol, 18:1 DG, 18:1-2:0 DG, dilinolein, tributyrin, tricaproin, trioctanoin, tricaprin, trilaurin, trimyristin, 15:0-18:1-15:0 TG, 16:0-(12-PAHSA)-18:1 TG, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid having a structure according to Formula CAT-I or Formula CAT-II; (II) a glyceride or an acylglycol selected from the group consisting of monoolein, monolinolein, diolein, 18:1-2:0 DG, dilinolein, tributyrin, tricaproin, trioctanoin, tricaprin, trilaurin, trimyristin, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid having a structure according to Formula CAT-I; (II) a glyceride or an acylglycol selected from the group consisting of monoolein, monolinolein, diolein, 18:1-2:0 DG, dilinolein, tributyrin, tricaproin, trioctanoin, tricaprin, trilaurin, trimyristin, tristearin, triolein, trilinolein, glyceryl trinonadecanoate, triarachidin, tripalmitin and a combination thereof, (III) a structural lipid; (IV) a helper lipid; and (V) a stealth lipid.

In some embodiments, the LNP comprises (I) a cationic lipid having a structure according to Formula CAT-II; (II) a glyceride or an acylglycol selected from the group consisting of monoolein, monolinolein, diolein, 18:1-2:0 DG, dilinolein, tributyrin, tricaproin, trioctanoin, tricaprin, trilaurin, trimyristin, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid having a structure according to Formula CAT-I or Formula CAT-II; (II) a glyceride or an acylglycol selected from the group consisting of monoolein, monolinolein, diolein, 18:1-2:0 DG, dilinolein, tricaprin, trilaurin, trimyristin, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid having a structure according to Formula CAT-I; (II) a glyceride or an acylglycol selected from the group consisting of monoolein, monolinolein, diolein, 18:1-2:0 DG, dilinolein, tricaprin, trilaurin, trimyristin, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid having a structure according to Formula CAT-II; (II) a glyceride or an acylglycol selected from the group consisting of monoolein, monolinolein, diolein, 18:1-2:0 DG, dilinolein, tricaprin, trilaurin, trimyristin, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid having a structure according to Formula CAT-I or Formula CAT-II; (II) a glyceride or an acylglycol selected from the group consisting of monoolein, monolinolein, diolein, tricaprin, trimyristin, tristearin, triolein, triarachidin, and a combination thereof; (III) a structural lipid; (IV) a helper lipid; and (V) a stealth lipid.

In some embodiments, the LNP comprises (I) a cationic lipid having a structure according to Formula CAT-I; (II) a glyceride or an acylglycol selected from the group consisting of monoolein, monolinolein, diolein, tricaprin, trimyristin, tristearin, triolein, triarachidin, and a combination thereof; (III) a structural lipid; (IV) a helper lipid; and (V) a stealth lipid.

In some embodiments, the LNP comprises (I) a cationic lipid having a structure according to Formula CAT-II; (II) a glyceride or an acylglycol selected from the group consiting of monoolein, monolinolein, diolein, tricaprin, trimyristin, tristearin, triolein, triarachidin, and a combination thereof; (III) a structural lipid; (IV) a helper lipid; and (V) a stealth lipid

In some embodiments, the LNP comprises (I) a cationic lipid having a structure according to Formula CAT-I or Formula CAT-II; (II) a glyceride or an acylglycol selected from the group consisting of diolein, tricaprin, triolein, and a combination thereof, (III) a structural lipid; (IV) a helper lipid; and (V) a stealth lipid.

In some embodiments, the LNP comprises (I) a cationic lipid having a structure according to Formula CAT-I; (II) a glyceride or an acylglycol selected from the group consisting of diolein, tricaprin, triolein, and a combination thereof; (III) a structural lipid; (IV) a helper lipid; and (V) a stealth lipid.

In some embodiments, the LNP comprises (I) a cationic lipid having a structure according to Formula CAT-II; (II) a glyceride or an acylglycol selected from the group consiting of diolein, tricaprin, triolein, and a combination thereof; (III) a structural lipid; (IV) a helper lipid; and (V) a stealth lipid.

In some embodiments, the LNP comprises (I) a cationic lipid that is cKK-E10, OF-02 or GL-HEPES-E3-E12-DS-4-E10; (II) a glyceride or an acylglycol selected from the group consisting of 1-C16 ether MG, monoolein, C18(plasm) MG, monolinolein, 08:0 DG, 10:0 DG, 12:0 DG, 14:0 DG, 15:0-18:1 DG, 16:0 ethylene glycol, 16:0 DG, 16:0-18:1 DG, 18:0 DG, diolein, 18:0-16:0 DG, 18:0-18:2 DG, 18:0-20:4 DG, 18:0-22:6 DG, 18:1 ethylene glycol, 18:1 DG, 18:1-2:0 DG, dilinolein, tributyrin, tricaproin, trioctanoin, tricaprin, trilaurin, trimyristin, 15:0-18:1-15:0 TG, 16:0-(12-PAHSA)-18:1 TG, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid that is cKK-E10, OF-02 or GL-HEPES-E3-E12-DS-4-E10; (II) a glyceride or an acylglycol selected from the group consisting of monoolein, monolinolein, diolein, 18:1-2:0 DG, dilinolein, tributyrin, tricaproin, trioctanoin, tricaprin, trilaurin, trimyristin, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid that is cKK-E10, OF-02 or GL-HEPES-E3-E12-DS-4-E10; (II) a glyceride or an acylglycol selected from the group consisting of monoolein, monolinolein, diolein, 18:1-2:0 DG, dilinolein, tricaprin, trilaurin, trimyristin, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid that is cKK-E10, OF-02 or GL-HEPES-E3-E12-DS-4-E10; (II) a glyceride or an acylglycol selected from the group consisting of monoolein, monolinolein, diolein, tricaprin, trimyristin, tristearin, triolein, triarachidin, and a combination thereof, (III) a structural lipid; (IV) a helper lipid; and (V) a stealth lipid.

In some embodiments, the LNP comprises (I) a cationic lipid that is cKK-E10, OF-02 or GL-HEPES-E3-E12-DS-4-E10; (II) a glyceride or an acylglycol selected from the group consisting of diolein, tricaprin, triolein, and a combination thereof; (III) a structural lipid; (IV) a helper lipid; and (V) a stealth lipid.

In some embodiments, the LNP comprises (I) GL-HEPES-E3-E12-DS-4-E10; (II) a glyceride or an acylglycol selected from the group consisting of 1-C16 ether MG, monoolein, C18(plasm) MG, monolinolein, 08:0 DG, 10:0 DG, 12:0 DG, 14:0 DG, 15:0-18:1 DG, 16:0 ethylene glycol, 16:0 DG, 16:0-18:1 DG, 18:0 DG, diolein, 18:0-16:0 DG, 18:0-18:2 DG, 18:0-20:4 DG, 18:0-22:6 DG, 18:1 ethylene glycol, 18:1 DG, 18:1-2:0 DG, dilinolein, tributyrin, tricaproin, trioctanoin, tricaprin, trilaurin, trimyristin, 15:0-18:1-15:0 TG, 16:0-(12-PAHSA)-18:1 TG, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) GL-HEPES-E3-E12-DS-4-E10; (II) a glyceride or an acylglycol selected from the group consisting of monoolein, monolinolein, diolein, 18:1-2:0 DG, dilinolein, tributyrin, tricaproin, trioctanoin, tricaprin, trilaurin, trimyristin, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) GL-HEPES-E3-E12-DS-4-E10; (II) a glyceride or an acylglycol selected from the group consisting of monoolein, monolinolein, diolein, 18:1-2:0 DG, dilinolein, tricaprin, trilaurin, trimyristin, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) GL-HEPES-E3-E12-DS-4-E10; (II) a glyceride or an acylglycol selected from the group consisting of diolein, dilinolein, trimyristin, tristearin, triarachidin, and a combination thereof; (III) a structural lipid; (IV) a helper lipid; and (V) a stealth lipid.

In some embodiments, the LNP comprises (I) GL-HEPES-E3-E12-DS-4-E10; (II) a glyceride or an acylglycol selected from the group consisting of trimyristin, tristearin, and a combination thereof, and (III) one or more lipids selected from the group consisting of a structural lipid, a helper lipid, and a stealth lipid.

In some embodiments, the LNP comprises (I) GL-HEPES-E3-E12-DS-4-E10; (II) a glyceride or an acylglycol selected from the group consisting of trimyristin, tristearin, and a combination thereof; (III) a structural lipid; (IV) a helper lipid; and (V) a stealth lipid.

In some embodiments, the LNP comprises (I) GL-HEPES-E3-E12-DS-4-E10; (II) trimyristin; and (III) one or more lipids selected from the group consisting of a structural lipid, a helper lipid, and a stealth lipid.

In some embodiments, the LNP comprises (I) GL-HEPES-E3-E12-DS-4-E10; (II) trimyristin; (III) a structural lipid; (IV) a helper lipid; and (V) a stealth lipid.

In some embodiments, the LNP comprises (I) GL-HEPES-E3-E12-DS-4-E10; (II) a glyceride or an acylglycol selected from the group consisting of 1-C16 ether MG, monoolein, C18(plasm) MG, monolinolein, 08:0 DG, 10:0 DG, 12:0 DG, 14:0 DG, 15:0-18:1 DG, 16:0 ethylene glycol, 16:0 DG, 16:0-18:1 DG, 18:0 DG, diolein, 18:0-16:0 DG, 18:0-18:2 DG, 18:0-20:4 DG, 18:0-22:6 DG, 18:1 ethylene glycol, 18:1 DG, 18:1-2:0 DG, dilinolein, tributyrin, tricaproin, trioctanoin, tricaprin, trilaurin, trimyristin, 15:0-18:1-15:0 TG, 16:0-(12-PAHSA)-18:1 TG, tristearin, triolein, trilinolein, glyceryl trinonadecanoate, triarachidin, tripalmitin and a combination thereof, (III) cholesterol; (IV) DOPE; and (V) DMG-PEG2000.

In some embodiments, the LNP comprises (I) GL-HEPES-E3-E12-DS-4-E10; (II) a glyceride or an acylglycol selected from the group consisting of monoolein, monolinolein, diolein, 18:1-2:0 DG, dilinolein, tributyrin, tricaproin, trioctanoin, tricaprin, trilaurin, trimyristin, tristearin, triolein, trilinolein, glyceryl trinonadecanoate, triarachidin, tripalmitin and a combination thereof, (III) cholesterol; (IV) DOPE; and (V) DMG-PEG2000.

In some embodiments, the LNP comprises (I) GL-HEPES-E3-E12-DS-4-E10; (II) a glyceride or an acylglycol selected from the group consisting of monoolein, monolinolein, diolein, 18:1-2:0 DG, dilinolein, tricaprin, trilaurin, trimyristin, tristearin, triolein, trilinolein, glyceryl trinonadecanoate, triarachidin, tripalmitin and a combination thereof; (III) cholesterol; (IV) DOPE; and (V) DMG-PEG2000.

In some embodiments, the LNP comprises (I) GL-HEPES-E3-E12-DS-4-E10; (II) a glyceride or an acylglycol selected from the group consisting of diolein, dilinolein, trimyristin, tristearin, triarachidin, and a combination thereof; (III) cholesterol; (IV) DOPE; and (V) DMG-PEG2000.

In some embodiments, the LNP comprises (I) GL-HEPES-E3-E12-DS-4-E10; (II) trimyristin; (III) cholesterol; (IV) DOPE; and (V) DMG-PEG2000.

In some embodiments, the LNP comprises (I) a cationic lipid that is TL1-12D-DMA, HEP-E4-E12, BAL-020, or BAL-005; (II) a glyceride or an acylglycol selected from the group consisting of 1-C16 ether MG, monoolein, C18(plasm) MG, monolinolein, 08:0 DG, 10:0 DG, 12:0 DG, 14:0 DG, 15:0-18:1 DG, 16:0 ethylene glycol, 16:0 DG, 16:0-18:1 DG, 18:0 DG, diolein, 18:0-16:0 DG, 18:0-18:2 DG, 18:0-20:4 DG, 18:0-22:6 DG, 18:1 ethylene glycol, 18:1 DG, 18:1-2:0 DG, dilinolein, tributyrin, tricaproin, trioctanoin, tricaprin, trilaurin, trimyristin, 15:0-18:1-15:0 TG, 16:0-(12-PAHSA)-18:1 TG, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid that is TL1-12D-DMA, HEP-E4-E12, BAL-020, or BAL-005; (II) a glyceride or an acylglycol selected from the group consisting of monoolein, monolinolein, diolein, 18:1-2:0 DG, dilinolein, tributyrin, tricaproin, trioctanoin, tricaprin, trilaurin, trimyristin, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid that is TL1-12D-DMA, HEP-E4-E12, BAL-020, or BAL-005; (II) a glyceride or an acylglycol selected from the group consisting of monoolein, monolinolein, diolein, 18:1-2:0 DG, dilinolein, tricaprin, trilaurin, trimyristin, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid that is TL1-12D-DMA, HEP-E4-E12, BAL-020, or BAL-005; (II) a glyceride or an acylglycol selected from the group consisting of monoolein, monolinolein, diolein, tricaprin, trimyristin, tristearin, triolein, triarachidin, and a combination thereof, (III) a structural lipid; (IV) a helper lipid; and (V) a stealth lipid.

In some embodiments, the LNP comprises (I) a cationic lipid that is TL1-12D-DMA, HEP-E4-E12, BAL-020, or BAL-005; (II) a glyceride or an acylglycol selected from the group consisting of diolein, tricaprin, triolein, and a combination thereof; (III) a structural lipid; (IV) a helper lipid; and (V) a stealth lipid.

In some embodiments, the LNP comprises (I) a cationic lipid having a structure according to Formula CAT-V; (II) a glyceride or an acylglycol; and (III) one or more lipids selected from the group consisting of (a) a structural lipid, (b) a helper lipid, and (c) a stealth lipid.

In some embodiments, the LNP comprises (I) a cationic lipid having a structure according to Formula CAT-V; (II) a glyceride or an acylglycol having a structure according to Formula I, Formula Ia, Formula Ib, Formula Ic, Formula Id, Formula Je, Formula II, Formula IIa, or Formula IIb; (III) a structural lipid; (IV) a helper lipid; and (V) a stealth lipid.

In some embodiments, the LNP comprises (I) a cationic lipid having a structure according to Formula CAT-V; (II) a glyceride or an acylglycol having a structure according to Formula I, Formula Ia, Formula Ib, Formula Ic, Formula Id, Formula Ie, Formula II, Formula IIa, or Formula IIb; (III) a structural lipid; (IV) a helper lipid; and (V) a stealth lipid.

In some embodiments, the LNP comprises (I) a cationic lipid having a structure according to Formula CAT-V; (II) a glyceride or an acylglycol selected from the group consisting of 1-C16 ether MG, monoolein, C18(plasm) MG, monolinolein, 08:0 DG, 10:0 DG, 12:0 DG, 14:0 DG, 15:0-18:1 DG, 16:0 ethylene glycol, 16:0 DG, 16:0-18:1 DG, 18:0 DG, diolein, 18:0-16:0 DG, 18:0-18:2 DG, 18:0-20:4 DG, 18:0-22:6 DG, 18:1 ethylene glycol, 18:1 DG, 18:1-2:0 DG, dilinolein, tributyrin, tricaproin, trioctanoin, tricaprin, trilaurin, trimyristin, 15:0-18:1-15:0 TG, 16:0-(12-PAHSA)-18:1 TG, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid having a structure according to Formula CAT-V; (II) a glyceride or an acylglycol selected from the group consisting of monoolein, monolinolein, diolein, 18:1-2:0 DG, dilinolein, tributyrin, tricaproin, trioctanoin, tricaprin, trilaurin, trimyristin, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid having a structure according to Formula CAT-V; (II) a glyceride or an acylglycol selected from the group consisting of monoolein, diolein, dilinolein, tricaprin, trimyristin, tristearin, triolein, trilinolein, glyceryl trinonadecanoate, triarachidin, and a combination thereof; (III) a structural lipid; (IV) a helper lipid; and (V) a stealth lipid.

In some embodiments, the LNP comprises (I) a cationic lipid having a structure according to Formula CAT-V; (II) a glyceride or an acylglycol selected from the group consisting of diolein, tricaprin, trimyristin, tristearin, triolein, trilinolein, glyceryl trinonadecanoate, triarachidin, and a combination thereof; (III) a structural lipid; (IV) a helper lipid; and (V) a stealth lipid.

In some embodiments, the LNP comprises (I) a cationic lipid having a structure according to Formula CAT-V; (II) a glyceride or an acylglycol selected from the group consisting of tricaprin, trimyristin, triolein, glyceryl trinonadecanoate, triarachidin, and a combination thereof; (III) a structural lipid; (IV) a helper lipid; and (V) a stealth lipid.

In some embodiments, the LNP comprises (I) a cationic lipid that is IM-001 or IS-001; (II) a glyceride or an acylglycol having a structure according to Formula I, Formula Ia, Formula Ib, Formula Ic, Formula Id, Formula Je, Formula II, Formula IIa, or Formula IIb; (III) a structural lipid; (IV) a helper lipid; and (V) a stealth lipid. In some embodiments, the LNP comprises (I) a cationic lipid having a structure according to Formula CAT-V; (II) a glyceride or an acylglycol having a structure according to Formula I, Formula Ia, Formula Ib, Formula Ic, Formula Id, Formula Ie, Formula II, Formula IIa, or Formula IIb; (III) a structural lipid; (IV) a helper lipid; and (V) a stealth lipid.

In some embodiments, the LNP comprises (I) a cationic lipid that is IM-001 or IS-001; (II) a glyceride or an acylglycol selected from the group consisting of 1-C16 ether MG, monoolein, C18(plasm) MG, monolinolein, 08:0 DG, 10:0 DG, 12:0 DG, 14:0 DG, 15:0-18:1 DG, 16:0 ethylene glycol, 16:0 DG, 16:0-18:1 DG, 18:0 DG, diolein, 18:0-16:0 DG, 18:0-18:2 DG, 18:0-20:4 DG, 18:0-22:6 DG, 18:1 ethylene glycol, 18:1 DG, 18:1-2:0 DG, dilinolein, tributyrin, tricaproin, trioctanoin, tricaprin, trilaurin, trimyristin, 15:0-18:1-15:0 TG, 16:0-(12-PAHSA)-18:1 TG, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid that is IM-001 or IS-001; (II) a glyceride or an acylglycol selected from the group consisting of monoolein, monolinolein, diolein, 18:1-2:0 DG, dilinolein, tributyrin, tricaproin, trioctanoin, tricaprin, trilaurin, trimyristin, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid that is IM-001 or IS-001; (II) a glyceride or an acylglycol selected from the group consisting of monoolein, diolein, dilinolein, tricaprin, trimyristin, tristearin, triolein, trilinolein, glyceryl trinonadecanoate, triarachidin, and a combination thereof; (III) a structural lipid; (IV) a helper lipid; and (V) a stealth lipid.

In some embodiments, the LNP comprises (I) a cationic lipid that is IM-001 or IS-001; (II) a glyceride or an acylglycol selected from the group consisting of diolein, tricaprin, trimyristin, tristearin, triolein, trilinolein, glyceryl trinonadecanoate, triarachidin, and a combination thereof; (III) a structural lipid; (IV) a helper lipid; and (V) a stealth lipid.

In some embodiments, the LNP comprises (I) a cationic lipid that is IM-001 or IS-001; (II) a glyceride or an acylglycol selected from the group consisting of tricaprin, trimyristin, triolein, glyceryl trinonadecanoate, triarachidin, and a combination thereof; (III) a structural lipid; (IV) a helper lipid; and (V) a stealth lipid.

In some embodiments, the LNP comprises (I) a cationic lipid having a structure according to Formula CAT-VI; (II) a glyceride or an acylglycol; and (III) one or more lipids selected from the group consisting of (a) a structural lipid, (b) a helper lipid, and (c) a stealth lipid.

In some embodiments, the LNP comprises (I) a cationic lipid having a structure according to Formula CAT-VI; (II) a glyceride or an acylglycol having a structure according to Formula I, Formula Ia, Formula Ib, Formula Ic, Formula Id, Formula Je, Formula II, Formula IIa, or Formula IIb; (III) a structural lipid; (IV) a helper lipid; and (V) a stealth lipid. In some embodiments, the LNP comprises (I) a cationic lipid having a structure according to Formula CAT-V; (II) a glyceride or an acylglycol having a structure according to Formula I, Formula Ia, Formula Ib, Formula Ic, Formula Id, Formula Ie, Formula II, Formula IIa, or Formula IIb; (III) a structural lipid; (IV) a helper lipid; and (V) a stealth lipid.

In some embodiments, the LNP comprises (I) a cationic lipid having a structure according to Formula CAT-VI; (II) a glyceride or an acylglycol selected from the group consisting of 1-C16 ether MG, monoolein, C18(plasm) MG, monolinolein, 08:0 DG, 10:0 DG, 12:0 DG, 14:0 DG, 15:0-18:1 DG, 16:0 ethylene glycol, 16:0 DG, 16:0-18:1 DG, 18:0 DG, diolein, 18:0-16:0 DG, 18:0-18:2 DG, 18:0-20:4 DG, 18:0-22:6 DG, 18:1 ethylene glycol, 18:1 DG, 18:1-2:0 DG, dilinolein, tributyrin, tricaproin, trioctanoin, tricaprin, trilaurin, trimyristin, 15:0-18:1-15:0 TG, 16:0-(12-PAHSA)-18:1 TG, tristearin, triolein, trilinolein, glyceryl trinonadecanoate, triarachidin, tripalmitin and a combination thereof, (III) a structural lipid; (IV) a helper lipid; and (V) a stealth lipid.

In some embodiments, the LNP comprises (I) a cationic lipid having a structure according to Formula CAT-VI; (II) a glyceride or an acylglycol selected from the group consisting of monoolein, monolinolein, diolein, 18:1-2:0 DG, dilinolein, tributyrin, tricaproin, trioctanoin, tricaprin, trilaurin, trimyristin, tristearin, triolein, trilinolein, glyceryl trinonadecanoate, triarachidin, tripalmitin and a combination thereof, (III) a structural lipid; (IV) a helper lipid; and (V) a stealth lipid.

In some embodiments, the LNP comprises (I) a cationic lipid having a structure according to Formula CAT-VI; (II) a glyceride or an acylglycol selected from the group consisting of monoolein, diolein, dilinolein, tricaprin, trimyristin, tristearin, triolein, trilinolein, glyceryl trinonadecanoate, triarachidin, and a combination thereof; (III) a structural lipid; (IV) a helper lipid; and (V) a stealth lipid.

In some embodiments, the LNP comprises (I) a cationic lipid having a structure according to Formula CAT-VI; (II) a glyceride or an acylglycol selected from the group consisting of monoolein, diolein, tricaprin, trimyristin, tristearin, triolein, glyceryl trinonadecanoate, and a combination thereof, (III) a structural lipid; (IV) a helper lipid; and (V) a stealth lipid.

In some embodiments, the LNP comprises (I) a cationic lipid having a structure according to Formula CAT-VI; (II) a glyceride or an acylglycol selected from the group consisting of monoolein, diolein, tricaprin, triolein, and a combination thereof; (III) a structural lipid; (IV) a helper lipid; and (V) a stealth lipid.

In some embodiments, the LNP comprises (I) a cationic lipid that is A2H7iiT6; (II) a glyceride or an acylglycol having a structure according to Formula I, Formula Ia, Formula Ib, Formula Ic, Formula Id, Formula Ie, Formula II, Formula IIa, or Formula IIb; (III) a structural lipid; (IV) a helper lipid; and (V) a stealth lipid. In some embodiments, the LNP comprises (I) a cationic lipid having a structure according to Formula CAT-V; (II) a glyceride or an acylglycol having a structure according to Formula I, Formula Ia, Formula Ib, Formula Ic, Formula Id, Formula Ie, Formula II, Formula IIa, or Formula IIb; (III) a structural lipid; (IV) a helper lipid; and (V) a stealth lipid.

In some embodiments, the LNP comprises (I) a cationic lipid that is A2H7iiT6; (II) a glyceride or an acylglycol selected from the group consisting of 1-C16 ether MG, monoolein, C18(plasm) MG, monolinolein, 08:0 DG, 10:0 DG, 12:0 DG, 14:0 DG, 15:0-18:1 DG, 16:0 ethylene glycol, 16:0 DG, 16:0-18:1 DG, 18:0 DG, diolein, 18:0-16:0 DG, 18:0-18:2 DG, 18:0-20:4 DG, 18:0-22:6 DG, 18:1 ethylene glycol, 18:1 DG, 18:1-2:0 DG, dilinolein, tributyrin, tricaproin, trioctanoin, tricaprin, trilaurin, trimyristin, 15:0-18:1-15:0 TG, 16:0-(12-PAHSA)-18:1 TG, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid that is A2H7iiT6; (II) a glyceride or an acylglycol selected from the group consisting of monoolein, monolinolein, diolein, 18:1-2:0 DG, dilinolein, tributyrin, tricaproin, trioctanoin, tricaprin, trilaurin, trimyristin, tristearin, triolein, trilinolein, glyceryl trinonadecanoate, triarachidin, tripalmitin and a combination thereof; (III) a structural lipid; (IV) a helper lipid; and (V) a stealth lipid.

In some embodiments, the LNP comprises (I) a cationic lipid that is A2H7iiT6; (II) a glyceride or an acylglycol selected from the group consisting of monoolein, diolein, dilinolein, tricaprin, trimyristin, tristearin, triolein, trilinolein, glyceryl trinonadecanoate, triarachidin, and a combination thereof; (III) a structural lipid; (IV) a helper lipid; and (V) a stealth lipid.

In some embodiments, the LNP comprises (I) a cationic lipid that is A2H7iiT6; (II) a glyceride or an acylglycol selected from the group consisting of diolein, tricaprin, trimyristin, tristearin, triolein, trilinolein, glyceryl trinonadecanoate, triarachidin, and a combination thereof, (III) a structural lipid; (IV) a helper lipid; and (V) a stealth lipid.

In some embodiments, the LNP comprises (I) a cationic lipid that is A2H7iiT6; (II) a glyceride or an acylglycol selected from the group consisting of tricaprin, trimyristin, triolein, glyceryl trinonadecanoate, triarachidin, and a combination thereof; (III) a structural lipid; (IV) a helper lipid; and (V) a stealth lipid.

In some embodiments, the cationic lipid may comprise a molar ratio from about 1% to about 90%, about 2% to about 70%, about 5% to about 50%, about 10% to about 40% of the total lipid present in the lipid nanoparticle, or about 20% to about 70% of the total lipid present in the lipid nanoparticle. In some embodiments, the cationic lipid may comprise a molar ratio between 35% and 55% of the total lipid present in the lipid nanoparticle. In some embodiments, the cationic lipid may comprise a molar ratio of about 40% of the total lipid present in the lipid nanoparticle. In some embodiments, the cationic lipid may comprise a molar ratio of about 45% of the total lipid present in the lipid nanoparticle. In some embodiments, the cationic lipid may comprise a molar ratio of about 50% of the total lipid present in the lipid nanoparticle.

In some embodiments, the structural lipid may comprise a molar ratio from about 5% to about 90%, or about 10% to about 70% of the total lipid present in the lipid nanoparticle. In some embodiments, the structural lipid may comprise a molar ratio between 20% and 35% of the total lipid present in the lipid nanoparticle. In some embodiments, the structural lipid may comprise a molar ratio of about 25% of the total lipid present in the lipid nanoparticle. In some embodiments, the structural lipid may comprise a molar ratio of about 28.5% of the total lipid present in the lipid nanoparticle.

In some embodiments, the helper lipid and the glyceride or acylglycol may comprise a combined molar ratio from about 2% to about 90%, or about 5% to about 70% of the total lipid present in the lipid nanoparticle. In some embodiments, the helper lipid and the glyceride or acylglycol may comprise a combined molar ratio between 10% and 35% of the total lipid present in the lipid nanoparticle. In some embodiments, the helper lipid and the glyceride or acylglycol may comprise a combined molar ratio between 15% and 35% of the total lipid present in the lipid nanoparticle. In some embodiments, the helper lipid and the glyceride or acylglycol may comprise a combined molar ratio of about 30% of the total lipid present in the lipid nanoparticle.

In some embodiments, the helper lipid may comprise a molar ratio from about 2% to about 90%, or about 5% to about 70% of the total lipid present in the lipid nanoparticle. In some embodiments, the helper lipid may comprise a molar ratio between 10% and 35% of the total lipid present in the lipid nanoparticle. In some embodiments, the helper lipid may comprise a molar ratio between 15% and 35% of the total lipid present in the lipid nanoparticle. In some embodiments, the helper lipid may comprise a molar ratio of about 25% of the total lipid present in the lipid nanoparticle.

In some embodiments, the glyceride or acylglycol may comprise a molar ratio from about 1% to about 20%, about 1% to about 20%, about 1% to about 15%, or about 1% to about 10% of the total lipid present in the lipid nanoparticle. In some embodiments, the glyceride or acylglycol may comprise a molar ratio between 1% and 15% of the total lipid present in the lipid nanoparticle. In some embodiments, the glyceride or acylglycol may comprise a molar ratio between 1% and 10% of the total lipid present in the lipid nanoparticle. In some embodiments, the glyceride or acylglycol may comprise a molar ratio of about 5% of the total lipid present in the lipid nanoparticle.

In some embodiments, the stealth (e.g., PEGylated) lipid may comprise a molar ratio from about 0% to about 20%, about 0.5% to about 20%, about 1% to about 15%, or about 1% to about 10% of the total lipid present in the lipid nanoparticle. In some embodiments, the stealth (e.g., PEGylated) lipid may comprise a molar ratio between 0.25% and 2.75% of the total lipid present in the lipid nanoparticle. In some embodiments, the stealth (e.g., PEGylated) lipid may comprise a molar ratio between 0.25% and 8.75% of the total lipid present in the lipid nanoparticle. In some embodiments, the stealth (e.g., PEGylated) lipid may comprise a molar ratio of about 1.5% of the total lipid present in the lipid nanoparticle. In some embodiments, the stealth (e.g., PEGylated) lipid may comprise a molar ratio of about 3% of the total lipid present in the lipid nanoparticle.

In some embodiments, the LNP comprises the cationic lipid at a molar ratio between 35% and 45%; the structural lipid at a molar ratio between 20% and 35%, the stealth lipid at a molar ratio between 0.25% and 8.75%, and the helper lipid and the glyceride or acylglycol at a combined molar ratio between 10% and 35%. In some embodiments, the LNP comprises the cationic lipid at a molar ratio of about 40%; the structural lipid at a molar ratio of about 28.5%; the stealth lipid at a molar ratio of about 1.5%, and the helper lipid and glyceride or acylglycol at a combined molar ratio of about 30%.

In some embodiments, the LNP comprises GL-HEPES-E3-E12-DS-4-E10 at a molar ratio between 35% and 45%; cholesterol at a molar ratio between 20% and 35%, DMG-PEG2000 at a molar ratio between 0.25% and 8.75%, and DOPE and trimyristin at a combined molar ratio between 10% and 35%.

In some embodiments, the LNP comprises GL-HEPES-E3-E12-DS-4-E10 at a molar ratio between 35% and 45%; cholesterol at a molar ratio between 20% and 35%, DMG-PEG2000 at a molar ratio between 0.25% and 8.75%, DOPE at a molar ratio between 15% and 35%, and trimyristin at a molar ratio between 1% and 10%.

In some embodiments, the LNP comprises GL-HEPES-E3-E12-DS-4-E10 at a molar ratio of about 40%, cholesterol at a molar ratio of about 28.5%, DMG-PEG2000 at a molar ratio of about 1.5%, and DOPE and trimyristin at a combined molar ratio of about 30%.

In some embodiments, the LNP comprises GL-HEPES-E3-E12-DS-4-E10 at a molar ratio of about 40%, cholesterol at a molar ratio of about 28.5%, DMG-PEG2000 at a molar ratio of about 1.5%, DOPE at a molar ratio of about 25%, and trimyristin at a molar ratio of about 5%.

To calculate the actual amount of each lipid (including the glyceride or acylglycol) to be put into an LNP formulation, the molar amount of the cationic lipid is first determined based on a desired N/P ratio, where N is the number of nitrogen atoms in the cationic lipid and P is the number of phosphate groups in the mRNA to be transported by the LNP. Next, the molar amount of each of the other lipids is calculated based on the molar amount of the cationic lipid and the molar ratio selected. These molar amounts are then converted to weights using the molecular weight of each lipid.

### Active Ingredients of the LNPs

The active ingredient of the present LNP composition may be an mRNA that encodes a polypeptide of interest. In certain embodiments, the polypeptide is an antigen. In certain embodiments, the polypeptide is a therapeutic polypeptide. The therapeutic polypeptide may be an antibody (e.g., an antibody heavy chain or an antibody light chain. The therapeutic polypeptide may be an enzyme.

The mRNA molecule encapsulated by the present disclosure LNPs may comprise at least one ribonucleic acid (RNA) comprising an ORF encoding a polypeptide of interest. In certain embodiments, the mRNA further comprises at least one 5′ UTR, 3′ UTR, a poly(A) tail, and/or a 5′ cap.

### A. 5′ Cap

An mRNA 5′ cap can provide resistance to nucleases found in most eukaryotic cells and promote translation efficiency. Several types of 5′ caps are known. A 7-methylguanosine cap (also referred to as “m⁷G” or “Cap-0”), comprises a guanosine that is linked through a 5′-5′-triphosphate bond to the first transcribed nucleotide.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp, (5′(A,G(5′)ppp(5′)A, and G(5′)ppp(5′)G. Additional cap structures are described in U.S. Publication No. US 2016/0032356 and U.S. Publication No. US 2018/0125989, which are incorporated herein by reference.

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′)G (the ARCA cap); G(5′)ppp(5′)A; G(5′)ppp(5′)G; m7G(5′)ppp(5′)A; m7G(5′)ppp(5′)G; m7G(5′)ppp(5′)(2′OMeA)pG; m7G(5′)ppp(5′)(2′OMeA)pU; m7G(5′)ppp(5′)(2′OMeG)pG (New England BioLabs, Ipswich, MA; TriLink Biotechnologies). 5′-capping of modified RNA may be completed post-transcriptionally using a vaccinia virus capping enzyme to generate the Cap 0 structure: m7G(5′)ppp(5′)G. Cap 1 structure may be generated using both vaccinia virus capping enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase.

In certain embodiments, the mRNA of the disclosure comprises a 5′ cap selected from the group consisting of 3′-O-Me-m7G(5′)ppp(5′)G (the ARCA cap), G(5′)ppp(5′)A, G(5′)ppp(5′)G, m7G(5′)ppp(5′)A, m7G(5′)ppp(5′)G, m7G(5′)ppp(5′)(2′OMeA)pG, m7G(5′)ppp(5′)(2′OMeA)pU, and m7G(5′)ppp(5′)(2′OMeG)pG.

In certain embodiments, the mRNA of the disclosure comprises a 5′ cap of:

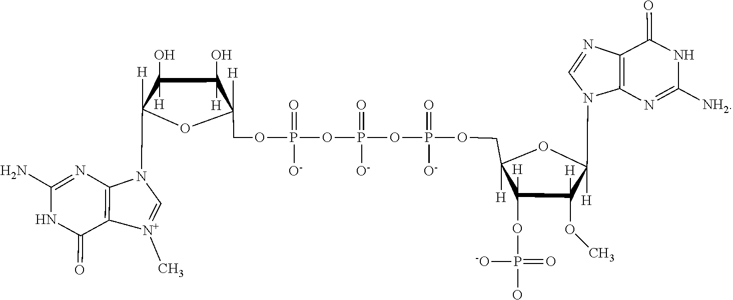

### B. Untranslated Region (UTR)

In some embodiments, the mRNA of the disclosure includes a 5′ and/or 3′ untranslated region (UTR). In mRNA, the 5′ UTR starts at the transcription start site and continues to the start codon but does not include the start codon. The 3′ UTR starts immediately following the stop codon and continues until the transcriptional termination signal.

In some embodiments, the mRNA disclosed herein may comprise a 5′ UTR that includes one or more elements that affect an mRNA's stability or translation. In some embodiments, a 5′ UTR may be about 10 to 5,000 nucleotides in length. In some embodiments, a 5′ UTR may be about 50 to 500 nucleotides in length. In some embodiments, the 5′ UTR is at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length or about 5,000 nucleotides in length.

In some embodiments, the mRNA disclosed herein may comprise a 3′ UTR comprising one or more of a polyadenylation signal, a binding site for proteins that affect an mRNA's stability of location in a cell, or one or more binding sites for miRNAs. In some embodiments, a 3′ UTR may be 50 to 5,000 nucleotides in length or longer. In some embodiments, a 3′ UTR may be 50 to 1,000 nucleotides in length or longer. In some embodiments, the 3′ UTR is at least about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 5′ or 3′ UTR that is derived from a gene distinct from the one encoded by the mRNA transcript (i.e., the UTR is a heterologous UTR).

In certain embodiments, the 5′ and/or 3′ UTR sequences can be derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Generally, these modifications improve the stability and/or pharmacokinetic properties (e.g., half-life) of the mRNA relative to their unmodified counterparts, and include, for example, modifications made to improve such mRNA resistance to in vivo nuclease digestion.

Exemplary 5′ UTRs include a sequence derived from a CMV immediate-early 1 (IE1) gene (U.S. Publication Nos. 2014/0206753 and 2015/0157565, each of which is incorporated herein by reference), or the sequence GGGAUCCUACC (U.S. Publication No. 2016/0151409, incorporated herein by reference).

In various embodiments, the 5′ UTR may be derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of an hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S. Publication No. 2016/0166710, supra).

In some embodiments, an internal ribosome entry site (IRES) is used instead of a 5′ UTR.

In some embodiments, the 5′UTR comprises a nucleic acid sequence reproduced below:

GGACAGAUCGCCUGGAGACGCCAUCCACGCUGUUUUGACCUCCAUAGAAGA
CACCGGGACCGAUCCAGCCUCCGCGGCCGGGAACGGUGCAUUGGAACGCGG
AUUCCCCGUGCCAAGAGUGACUCACCGUCCUUGACACG.

In some embodiments, the 3′UTR comprises a nucleic acid sequence reproduced below:

CGGGUGGCAUCCCUGUGACCCCUCCCCAGUGCCUCUCCUGGCCCUGGAAGU
UGCCACUCCAGUGCCCACCAGCCUUGUCCUAAUAAAAUUAAGUUGCAUC.

The 5′ UTR and 3′UTR are described in further detail in WO2012/075040, incorporated herein by reference.

### C. Polyadenylated Tail

As used herein, the terms “poly(A) sequence,” “poly(A) tail,” and “poly(A) region” refer to a sequence of adenosine nucleotides at the 3′ end of the mRNA molecule. The poly(A) tail may confer stability to the mRNA and protect it from exonuclease degradation. The poly(A) tail may enhance translation. In some embodiments, the poly(A) tail is essentially homopolymeric. For example, a poly(A) tail of 100 adenosine nucleotides may have essentially a length of 100 nucleotides. In certain embodiments, the poly(A) tail may be interrupted by at least one nucleotide different from an adenosine nucleotide (e.g., a nucleotide that is not an adenosine nucleotide). For example, a poly(A) tail of 100 adenosine nucleotides may have a length of more than 100 nucleotides (comprising 100 adenosine nucleotides and at least one nucleotide, or a stretch of nucleotides, that are different from an adenosine nucleotide). In certain embodiments, the poly(A) tail comprises the sequence

AAAAAAAAAAAAAAAAAAAAAAAAAAAAAAGCAUAUGACUAAAAAAAAA
AAAAAAAAAAAAAAAAAAAAAAAAAAAAAAAAAAAAAAAAAAAAAAAAA
AAAAAAAAAAAA.

The “poly(A) tail,” as used herein, typically relates to RNA. However, in the context of the disclosure, the term likewise relates to corresponding sequences in a DNA molecule (e.g., a “poly(T) sequence”).

The poly(A) tail may comprise about 10 to about 500 adenosine nucleotides, about 10 to about 200 adenosine nucleotides, about 40 to about 200 adenosine nucleotides, or about 40 to about 150 adenosine nucleotides. The length of the poly(A) tail may be at least about 10, 50, 75, 100, 150, 200, 250, 300, 350, 400, 450, or 500 adenosine nucleotides.

In some embodiments where the nucleic acid is an RNA, the poly(A) tail of the nucleic acid is obtained from a DNA template during RNA in vitro transcription. In certain embodiments, the poly(A) tail is obtained in vitro by common methods of chemical synthesis without being transcribed from a DNA template. In various embodiments, poly(A) tails are generated by enzymatic polyadenylation of the RNA (after RNA in vitro transcription) using commercially available polyadenylation kits and corresponding protocols, or alternatively, by using immobilized poly(A)polymerases, e.g., using methods and means as described in WO2016/174271.

The nucleic acid may comprise a poly(A) tail obtained by enzymatic polyadenylation, wherein the majority of nucleic acid molecules comprise about 100 (+/−20) to about 500 (+/−50) or about 250 (+/−20) adenosine nucleotides.

In some embodiments, the nucleic acid may comprise a poly(A) tail derived from a template DNA and may additionally comprise at least one additional poly(A) tail generated by enzymatic polyadenylation, e.g., as described in WO2016/091391.

In certain embodiments, the nucleic acid comprises at least one polyadenylation signal.

In various embodiments, the nucleic acid may comprise at least one poly(C) sequence.

The term “poly(C) sequence,” as used herein, is intended to be a sequence of cytosine nucleotides of up to about 200 cytosine nucleotides. In some embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In some embodiments, the poly(C) sequence comprises about 30 cytosine nucleotides.

### D. Chemical Modification

The mRNA disclosed herein may be modified or unmodified. In some embodiments, the mRNA may comprise at least one chemical modification. In some embodiments, the mRNA disclosed herein may contain one or more modifications that typically enhance RNA stability. Exemplary modifications can include backbone modifications, sugar modifications, or base modifications. In some embodiments, the disclosed mRNA may be synthesized from naturally occurring nucleotides and/or nucleotide analogues (modified nucleotides) including, but not limited to, purines (adenine (A) and guanine (G)) or pyrimidines (thymine (T), cytosine (C), and uracil (U)). In certain embodiments, the disclosed mRNA may be synthesized from modified nucleotide analogues or derivatives of purines and pyrimidines, such as, e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine.

In some embodiments, the disclosed mRNA may comprise at least one chemical modification including, but not limited to, pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In some embodiments, the chemical modification is selected from the group consisting of pseudouridine, N1-methylpseudouridine, 5-methylcytosine, 5-methoxyuridine, and a combination thereof.

In some embodiments, the chemical modification comprises N1-methylpseudouridine.

In some embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In some embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

The preparation of such analogues is described, e.g., in U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

E. mRNA Synthesis

The mRNAs disclosed herein may be synthesized according to any of a variety of methods. For example, mRNAs according to the present disclosure may be synthesized via in vitro transcription (IVT). Some methods for in vitro transcription are described, e.g., in Geall et al. (2013) Semin. Immunol. 25(2): 152-159; Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, an appropriate RNA polymerase (e.g., T3, T7, or SP6 RNA polymerase), DNase I, pyrophosphatase, and/or RNase inhibitor. The exact conditions may vary according to the specific application. The presence of these reagents is generally undesirable in a final mRNA product and these reagents can be considered impurities or contaminants which can be purified or removed to provide a clean and/or homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

Where desired, the LNP or the LNP formulation may be multi-valent. In some embodiments, the LNP may carry mRNAs that encode more than one polypeptide (e.g., antigen), such as two, three, four, five, six, seven, eight, nine, ten, or more polypeptides. For example, the LNP may carry multiple mRNA molecules, each encoding a different polypeptide; or carry a polycistronic mRNA that can be translated into more than one polypeptide (e.g., each polypeptide-coding sequence is separated by a nucleotide linker encoding a self-cleaving peptide such as a 2A peptide). An LNP carrying different mRNA molecules typically comprises (encapsulate) multiple copies of each mRNA molecule. For example, an LNP carrying or encapsulating two different mRNA molecules typically carries multiple copies of each of the two different mRNA molecules.

In some embodiments, a single LNP formulation may comprise multiple kinds (e.g., two, three, four, five, six, seven, eight, nine, ten, or more) of LNPs, each kind carrying a different mRNA.

### Buffer and Other Components

To stabilize the nucleic acid and/or LNPs (e.g., to prolong the shelf-life of the vaccine product), to facilitate administration of the LNP pharmaceutical composition, and/or to enhance in vivo expression of the nucleic acid, the nucleic acid and/or LNP can be formulated in combination with one or more carriers, targeting ligands, stabilizing reagents (e.g., preservatives and antioxidants), and/or other pharmaceutically acceptable excipients. Examples of such excipients are parabens, thimerosal, thiomersal, chlorobutanol, bezalkonium chloride, and chelators (e.g., EDTA).

The LNP compositions of the present disclosure can be provided as a frozen liquid form or a lyophilized form. A variety of cryoprotectants may be used, including, without limitations, sucrose, trehalose, glucose, mannitol, mannose, dextrose, and the like. The cryoprotectant may constitute 5-30% (w/v) of the LNP composition. In some embodiments, the LNP composition comprises trehalose, e.g., at 5-30% (e.g., 10%) (w/v). Once formulated with the cryoprotectant, the LNP compositions may be frozen (or lyophilized and cryopreserved) at −20° C. to −80° C.

The LNP compositions may be provided to a patient in an aqueous buffered solution—thawed if previously frozen, or if previously lyophilized, reconstituted in an aqueous buffered solution at bedside. The buffered solution may be isotonic and suitable for e.g., intramuscular or intradermal injection. In some embodiments, the buffered solution is a phosphate-buffered saline (PBS).

### Processes for Making the Present LNP Formulations

The present LNPs can be prepared by various techniques presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion that results in the formation of MLVs. Unilamellar vesicles (ULV) can then be formed by homogenization, sonication or extrusion of the multilamellar vesicles. In addition, unilamellar vesicles can be formed by detergent removal techniques.

Various methods are described in US 2011/0244026, US 2016/0038432, US 2018/0153822, US 2018/0125989, and PCT/US2020/043223 (filed Jul. 23, 2020) and can be used to practice the present invention. One exemplary process entails encapsulating mRNA by mixing it with a mixture of lipids, without first pre-forming the lipids into lipid nanoparticles, as described in US 2016/0038432. Another exemplary process entails encapsulating mRNA by mixing pre-formed LNPs with mRNA, as described in US 2018/0153822.

In some embodiments, the process of preparing mRNA-loaded LNPs includes a step of heating one or more of the solutions to a temperature greater than ambient temperature, the one or more solutions being the solution comprising the pre-formed lipid nanoparticles, the solution comprising the mRNA and the mixed solution comprising the LNP-encapsulated mRNA. In some embodiments, the process includes the step of heating one or both of the mRNA solution and the pre-formed LNP solution, prior to the mixing step. In some embodiments, the process includes heating one or more of the solutions comprising the pre-formed LNPs, the solution comprising the mRNA and the solution comprising the LNP-encapsulated mRNA, during the mixing step. In some embodiments, the process includes the step of heating the LNP-encapsulated mRNA, after the mixing step. In some embodiments, the temperature to which one or more of the solutions is heated is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature is about 65° C.

Various methods may be used to prepare an mRNA solution suitable for the present invention. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water or a buffer at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include but are not limited to gear pumps, peristaltic pumps and centrifugal pumps. Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of, or greater than, about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

The process of incorporation of a desired mRNA into a lipid nanoparticle is referred to as “loading.” Exemplary methods are described in Lasic et al., FEBS Lett. (1992) 312:255-8. The LNP-incorporated nucleic acids may be completely or partially located in the interior space of the lipid nanoparticle, within the bilayer membrane of the lipid nanoparticle, or associated with the exterior surface of the lipid nanoparticle membrane. The incorporation of an mRNA into lipid nanoparticles is also referred to herein as “encapsulation” wherein the nucleic acid is entirely or substantially contained within the interior space of the lipid nanoparticle.

Suitable LNPs may be made in various sizes. In some embodiments, decreased size of lipid nanoparticles is associated with more efficient delivery of an mRNA. Selection of an appropriate LNP size may take into consideration the site of the target cell or tissue and to some extent the application for which the lipid nanoparticle is being made.

A variety of methods known in the art are available for sizing of a population of lipid nanoparticles. Preferred methods herein utilize Zetasizer Nano ZS (Malvern Panalytical) to measure LNP particle size. In one protocol, 10 μl of an LNP sample are mixed with 990 μl of 10% trehalose. This solution is loaded into a cuvette and then put into the Zetasizer machine. The z-average diameter (nm), or cumulants mean, is regarded as the average size for the LNPs in the sample. The Zetasizer machine can also be used to measure the polydispersity index (PDI) by using dynamic light scattering (DLS) and cumulant analysis of the autocorrelation function. Average LNP diameter may be reduced by sonication of formed LNP. Intermittent sonication cycles may be alternated with quasi-elastic light scattering (QELS) assessment to guide efficient lipid nanoparticle synthesis.

In some embodiments, the majority of purified LNPs, i.e., greater than about 50%, 55%, 60%, 65%, 70%, 75%, 80%, 85%, 90%, 95%, 96%, 97%, 98%, or 99% of the LNPs, have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all (e.g., greater than 80 or 90%) of the purified lipid nanoparticles have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm).

In some embodiments, the LNPs in the present composition have an average size of less than 150 nm, less than 120 nm, less than 100 nm, less than 90 nm, less than 80 nm, less than 70 nm, less than 60 nm, less than 50 nm, less than 30 nm, or less than 20 nm.

In some embodiments, greater than about 70%, 75%, 80%, 85%, 90%, 95%, 96%, 97%, 98%, 99% of the LNPs in the present composition have a size ranging from about 40-90 nm (e.g., about 45-85 nm, about 50-80 nm, about 55-75 nm, about 60-70 nm), about 40-90 nm (e.g., about 45-85 nm, about 50-80 nm, about 55-75 nm, about 60-70 nm), or about 50-70 nm (e.g., 55-65 nm) are particular suitable for pulmonary delivery via nebulization.

In some embodiments, the dispersity, or measure of heterogeneity in size of molecules (PDI), of LNPs in a pharmaceutical composition provided by the present invention is less than about 0.5. In some embodiments, an LNP has a PDI of less than about 0.5, less than about 0.4, less than about 0.3, less than about 0.28, less than about 0.25, less than about 0.23, less than about 0.20, less than about 0.18, less than about 0.16, less than about 0.14, less than about 0.12, less than about 0.10, or less than about 0.08. The PDI may be measured by a Zetasizer machine as described above.

In some embodiments, greater than about 75%, 80%, 85%, 90%, 95%, 96%, 97%, 98%, or 99% of the purified LNPs in a pharmaceutical composition provided herein encapsulate an mRNA within each individual particle. In some embodiments, substantially all (e.g., greater than 80% or 90%) of the purified lipid nanoparticles in a pharmaceutical composition encapsulate an mRNA within each individual particle. In some embodiments, a lipid nanoparticle has an encapsulation efficiency of between 50% and 99%; or greater than about 60, 65, 70, 75, 80, 85, 90, 92, 95, 98, or 99%. Typically, lipid nanoparticles for use herein have an encapsulation efficiency of at least 90% (e.g., at least 91, 92, 93, 94, or 95%).

In some embodiments, an LNP has a N/P ratio of between 1 and 10. In some embodiments, a lipid nanoparticle has a N/P ratio above 1, about 1, about 2, about 3, about 4, about 5, about 6, about 7, or about 8. In further embodiments, a typical LNP herein has an N/P ratio of 4.

In some embodiments, a pharmaceutical composition according to the present invention contains at least about 0.5 μg, 1 μg, 5 μg, 10 μg, 100 μg, 500 μg, or 1000 μg of encapsulated mRNA. In some embodiments, a pharmaceutical composition contains about 0.1 μg to 1000 μg, at least about 0.5 μg, at least about 0.8 ag, at least about 1 μg, at least about 5 μg, at least about 8 μg, at least about 10 ag, at least about 50 μg, at least about 100 μg, at least about 500 μg, or at least about 1000 μg of encapsulated mRNA.

Packaging and Use of the mRNA-LNP

The mRNA-LNP can be packaged for parenteral (e.g., intramuscular, intradermal, subcutaneous, or intravenous) administration or nasopharyngeal (e.g., intranasal) administration. The compositions may be in the form of an extemporaneous formulation, where the LNP composition is lyophilized and reconstituted with a physiological buffer (e.g., PBS) just before use. The compositions also may be shipped and provided in the form of an aqueous solution or a frozen aqueous solution and can be directly administered to subjects without reconstitution (after thawing, if previously frozen).

Accordingly, the present disclosure provides an article of manufacture, such as a kit, that provides the mRNA-LNP in a single container, or provides the mRNA-LNP in one container and a physiological buffer for reconstitution in another container. The container(s) may contain a single-use dosage or multi-use dosage. The containers may be pre-treated glass vials or ampules. The article of manufacture may include instructions for use as well.

In some embodiments, the present invention provides methods of preventing or treating a disease or disorder by administering the composition of the invention to a subject in need thereof. In some embodiments, the subject is suffering from or susceptible to an infection.

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

### Particular Embodiments

In a first embodiment, a composition comprising a lipid nanoparticle (LNP) is provided, wherein the LNP comprises: (I) an ionizable lipid; (II) a glyceride or an acylglycol; and (III) one or more lipids selected from the group consisting of: (a) a structural lipid; (b) a helper lipid; and (c) a stealth lipid.

In a second embodiment, the LNP comprises: (I) an ionizable lipid; (II) a glyceride or an acylglycol; (III) a structural lipid; (IV) a helper lipid; and (V) a stealth lipid.

In a third embodiment, the glyceride or acylglycol is a monoglyceride, a diglyceride, a triglyceride, or a diacylglycol.

In a fourth embodiment, the glyceride or acylglycol has a structure according to Formula I or Formula II:

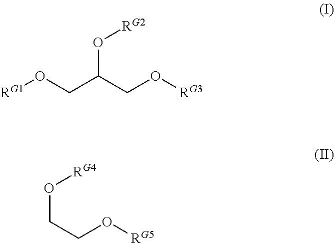

wherein:
R^{G1}, R^{G2}, and R^{G3} are each independently H, —C₍₁₋₂₅₎alkyl, —C₍₁₋₂₅₎alkenyl, —C(O)C₍₁₋₂₅₎alkyl, or —C(O)C₍₁₋₂₅₎alkenyl, wherein the —C₍₁₋₂₅₎alkyl, —C₍₁₋₂₅₎alkenyl, —C(O)C₍₁₋₂₅₎alkyl, and —C(O)C₍₁₋₂₅₎alkenyl are optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl;
provided that no more than two of R^{G1}, R^{G2} and R^{G3} are H;
R^{G4} is-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl; R^{G5} is H, —C₍₁₋₂₅₎alkyl, —C₍₁₋₂₅₎alkenyl, —C(O)C₍₁₋₂₅₎alkyl, or —C(O)C₍₁₋₂₅₎alkenyl, wherein the —C₍₁₋₂₅₎alkyl, —C₍₁₋₂₅₎alkenyl, —C(O)C₍₁₋₂₅₎alkyl, and —C(O)C₍₁₋₂₅₎alkenyl are optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In a fifth embodiment, the glyceride or acylglycol has a structure according to Formula I:

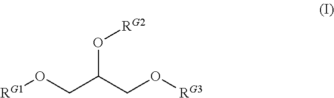

wherein:
R^{G1}, R^{G2}, and R^{G3} are each independently H, —C₍₁₋₂₅₎alkyl, —C₍₁₋₂₅₎alkenyl, —C(O)C₍₁₋₂₅₎alkyl, or —C(O)C₍₁₋₂₅₎alkenyl, wherein the —C₍₁₋₂₅₎alkyl, —C₍₁₋₂₅₎alkenyl, —C(O)C₍₁₋₂₅₎alkyl, and —C(O)C₍₁₋₂₅₎alkenyl are optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl;
provided that no more than two of R^{G1}, R^{G2} and R^{G3} are H.

In a sixth embodiment, the glyceride or acylglycol has a structure according to Formula Ia or Ib:

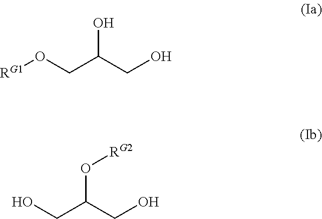

wherein R^{G1} and R^{G2} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In a seventh embodiment, the glyceride or acylglycol has a structure according to Formula Ia or Ib, wherein: R^{G1} and R^{G2} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl.

In an eighth embodiment, the glyceride or acylglycol has a structure according to Formula Ia or Ib, wherein: R^{G1} and R^{G2} are each independently-C₍₁₁₋₂₅₎alkyl, —C₍₁₁₋₂₅₎alkenyl, —C(O)C₍₁₁₋₂₅₎alkyl, or —C(O)C₍₁₁₋₂₅₎alkenyl.

In a ninth embodiment, the glyceride or acylglycol has a structure according to Formula Ic or Id:

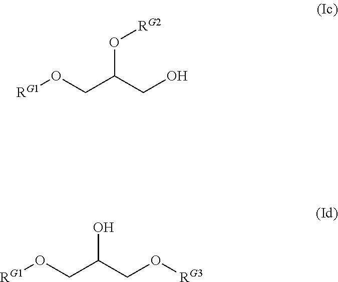

wherein R^{G1}, R^{G2}, and R^{G3} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In a tenth embodiment, the glyceride or acylglycol has a structure according to Formula Ic or Id, wherein: R^{G1}, R^{G2}, and R^{G3} are each independently —C(O)C₍₁₋₂₅₎alkyl or —C(O)C₍₁₋₂₅₎alkenyl.

In an 11^{th} embodiment, the glyceride or acylglycol has a structure according to Formula Ic or Id, wherein: R^{G1} is —C(O)C₍₃₋₂₅₎alkyl or —C(O)C₍₃₋₂₅₎alkenyl; R^{G2} is —C(O)C₍₁₋₂₅₎alkyl or —C(O)C₍₁₋₂₅₎alkenyl; and R^{G3} is —C(O)C₍₃₋₂₅₎alkyl or —C(O)C₍₃₋₂₅₎alkenyl.

In a 12^{th} embodiment, the glyceride or acylglycol has a structure according to Formula Ie:

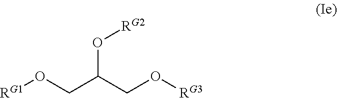

wherein R^{G1}, R^{G2}, and R^{G3} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In a 13^{th} embodiment, the glyceride or acylglycol has a structure according to Formula Ie, wherein R^{G1} is —C(O)C₍₁₋₂₅₎alkyl or —C(O)C₍₁₋₂₅₎alkenyl; R^{G2} is —C(O)C₍₁₋₂₅₎alkyl or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl; and R^{G3} is —C(O)C₍₁₋₂₅₎alkyl or —C(O)C₍₁₋₂₅₎alkenyl.

In a 14^{th} embodiment, the glyceride or acylglycol has a structure according to Formula Ie, wherein R^{G1}, R^{G2}, and R^{G3} are each independently —C(O)C₍₇₋₂₁₎alkyl or —C(O)C₍₇₋₂₁₎alkenyl.

In a 15^{th} embodiment, the glyceride or acylglycol has a structure according to Formula IIa or IIb:

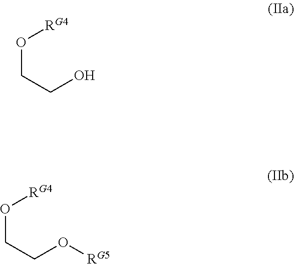

wherein R^{G4} and R^{G5} are each independently-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

In a 16^{th} embodiment, the glyceride or acylglycol has a structure according to Formula IIa or IIb, wherein R^{G4} and R^{G5} are each independently —C(O)C₍₁₋₂₅₎alkyl or —C(O)C₍₁₋₂₅₎alkenyl.

In a 17^{th} embodiment, the glyceride or acylglycol is hydrolyzable by lipase.

In an 18^{th} embodiment, the glyceride or acylglycol is selected from the group consisting of:

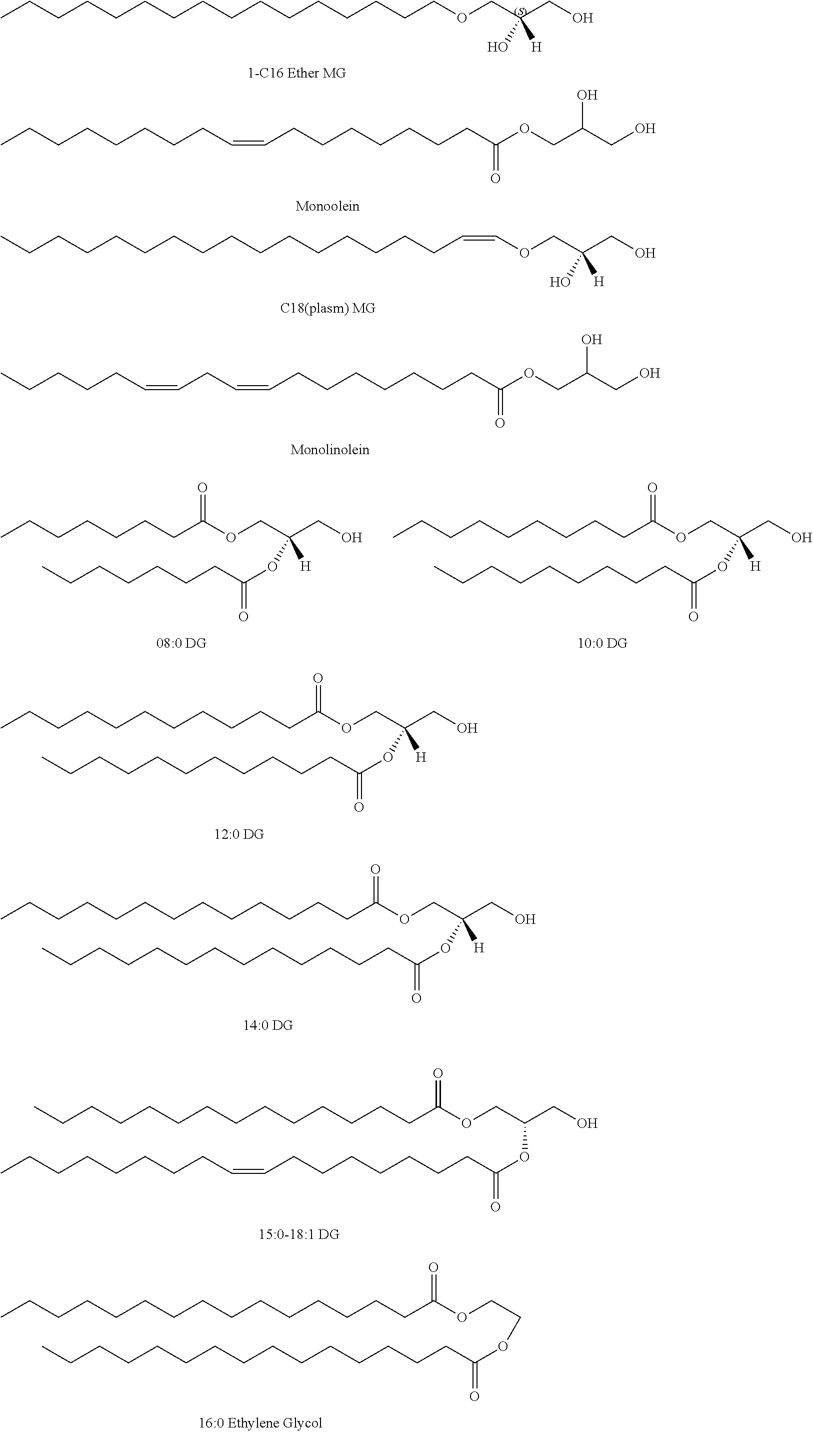

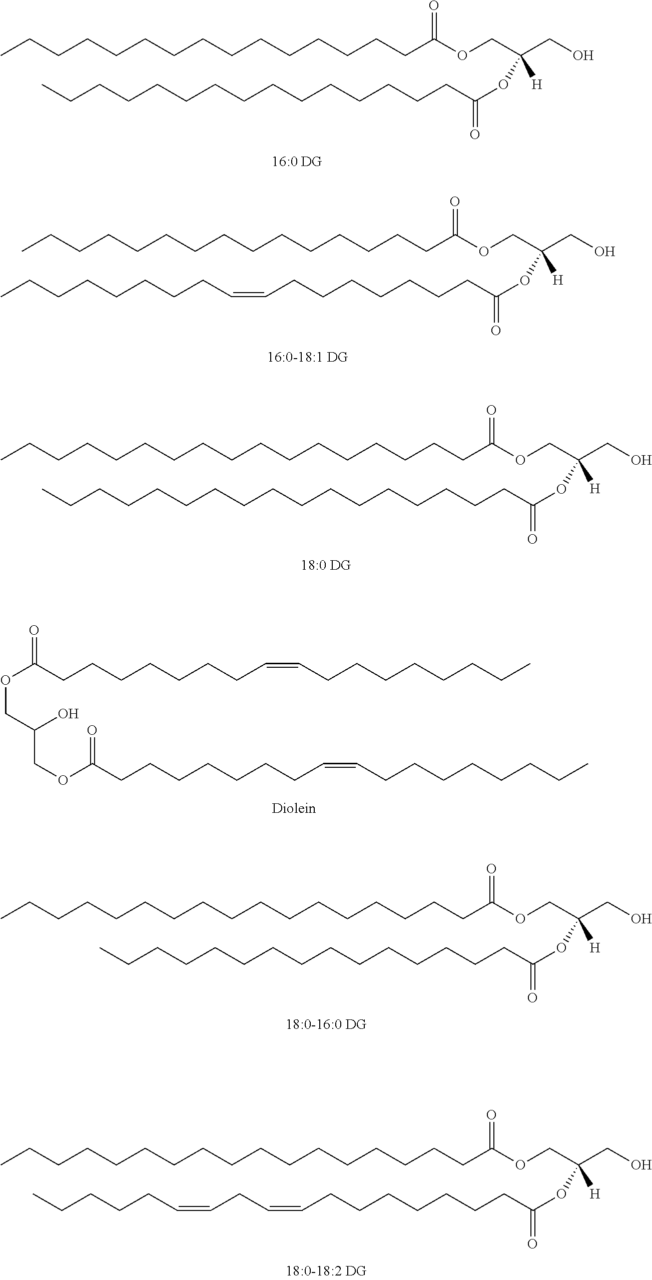

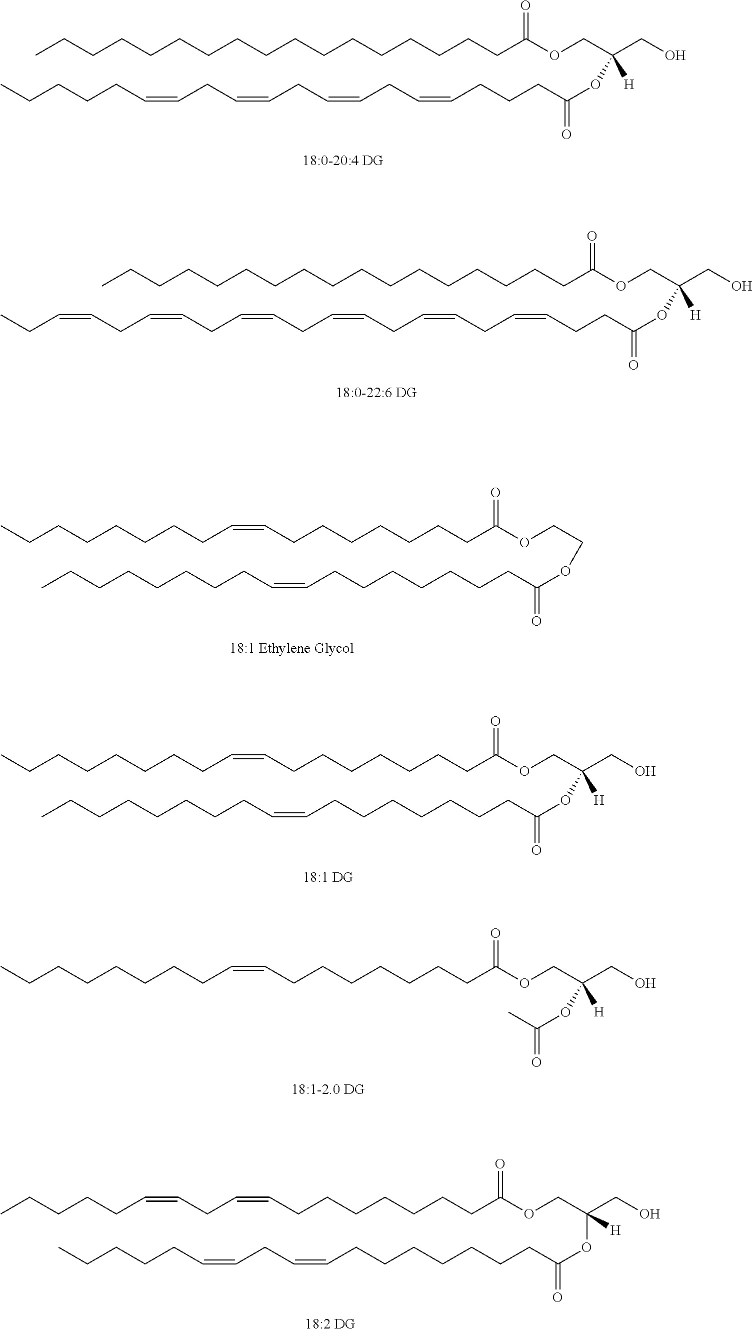 [SMILES: CCCCC/C=C\C/C=C\C/C=C\C/C=C\CCCC(=O)O[C@@H](CO)COC(=O)CCCCCCCCCCCCCCCCC ; CC/C=C\C/C=C\C/C=C\C/C=C\C/C=C\C/C=C\CCC(=O)O[C@@H](CO)COC(=O)CCCCCCCCCCCCCCCCC ; CCCCCCCC/C=C\CCCCCCCC(=O)OCCOC(=O)CCCCCCC/C=C\CCCCCCCC ; CCCCCCCC/C=C\CCCCCCCC(=O)OC[C@H](CO)OC(=O)CCCCCCC/C=C\CCCCCCCC ; CCCCCCCC/C=C\CCCCCCCC(=O)OC[C@H](CO)OC(C)=O ; CCCCC/C=C\C/C=C\CCCCCCCC(=O)O[C@@H](CO)COC(=O)CCCCCCC/C=C\C/C=C\C(C)CCCC]

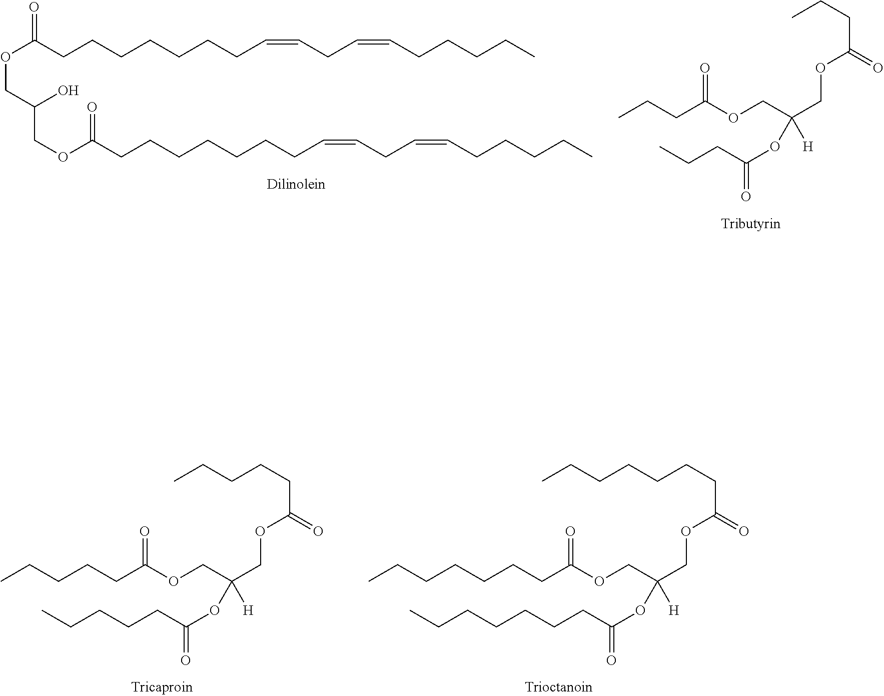

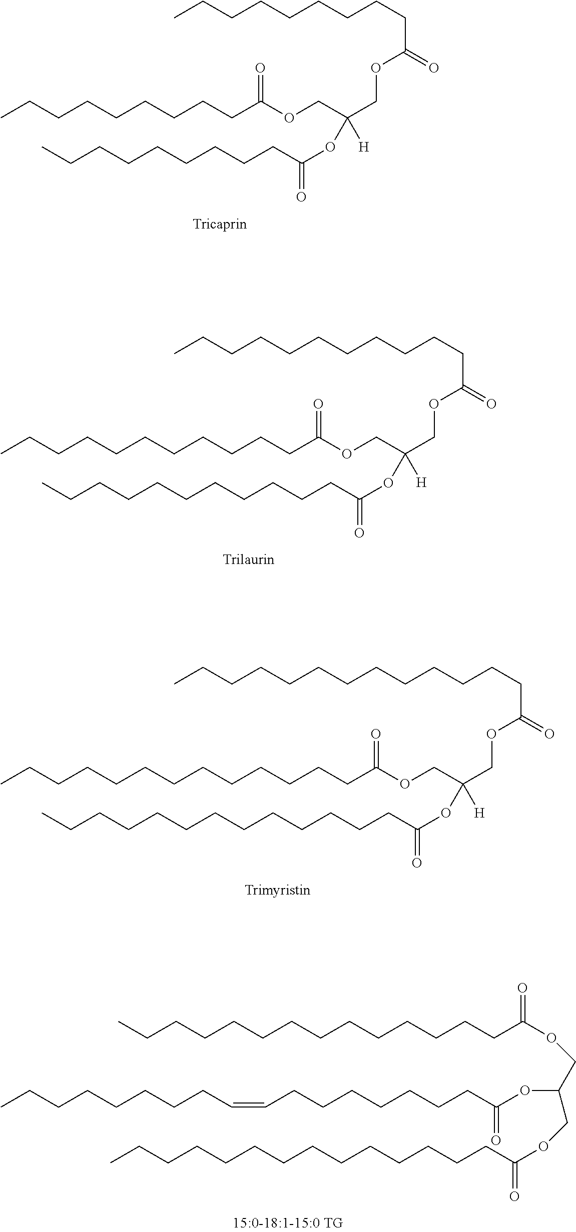 [SMILES: CCCCCCCCCC(=O)OCC(COC(=O)CCCCCCCCC)OC(=O)CCCCCCCCC ; CCCCCCCCCCCC(=O)OCC(COC(=O)CCCCCCCCCCC)OC(=O)CCCCCCCCCCC ; CCCCCCCCCCCCCC(=O)OCC(COC(=O)CCCCCCCCCCCCC)OC(=O)CCCCCCCCCCCCC ; CCCCCCCC/C=C\CCCCCCCC(=O)OC(COC(=O)CCCCCCCCCCCCCC)COC(=O)CCCCCCCCCCCCCC]

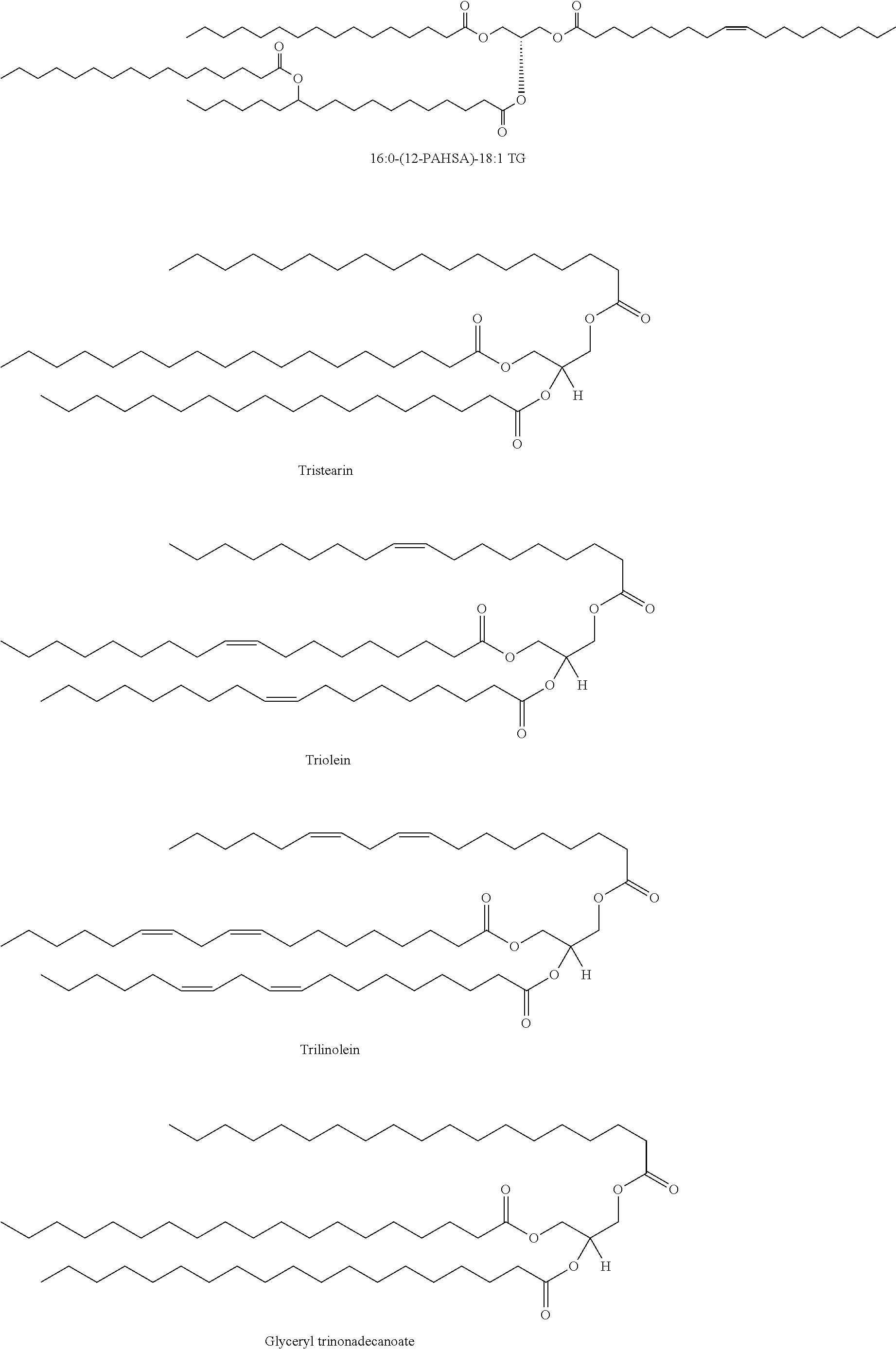

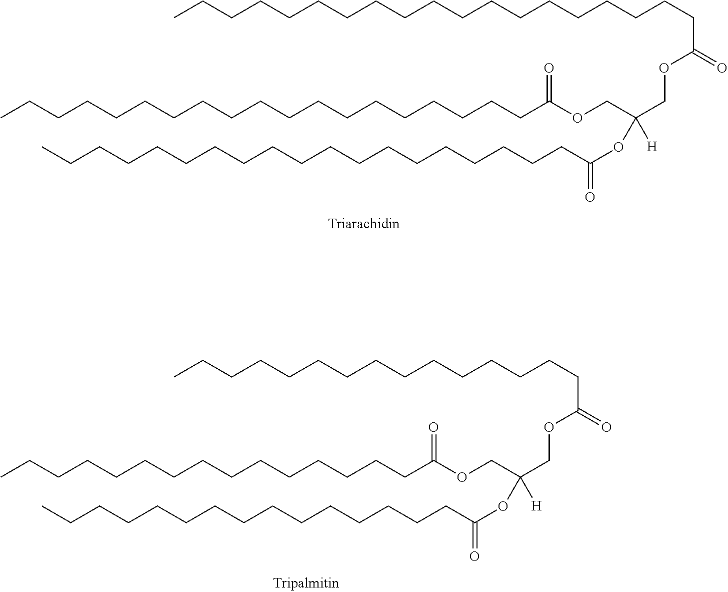 [SMILES: CCCCCCCCCCCCCCCCCCCC(=O)OCC(COC(=O)CCCCCCCCCCCCCCCCCCC)OC(=O)CCCCCCCCCCCCCCCCCCC ; CCCCCCCCCCCCCCCC(=O)OCC(COC(=O)CCCCCCCCCCCCCCC)OC(=O)CCCCCCCCCCCCCCC]

In a 19^{th} embodiment, the glyceride or acylglycol is selected from the group consisting of:

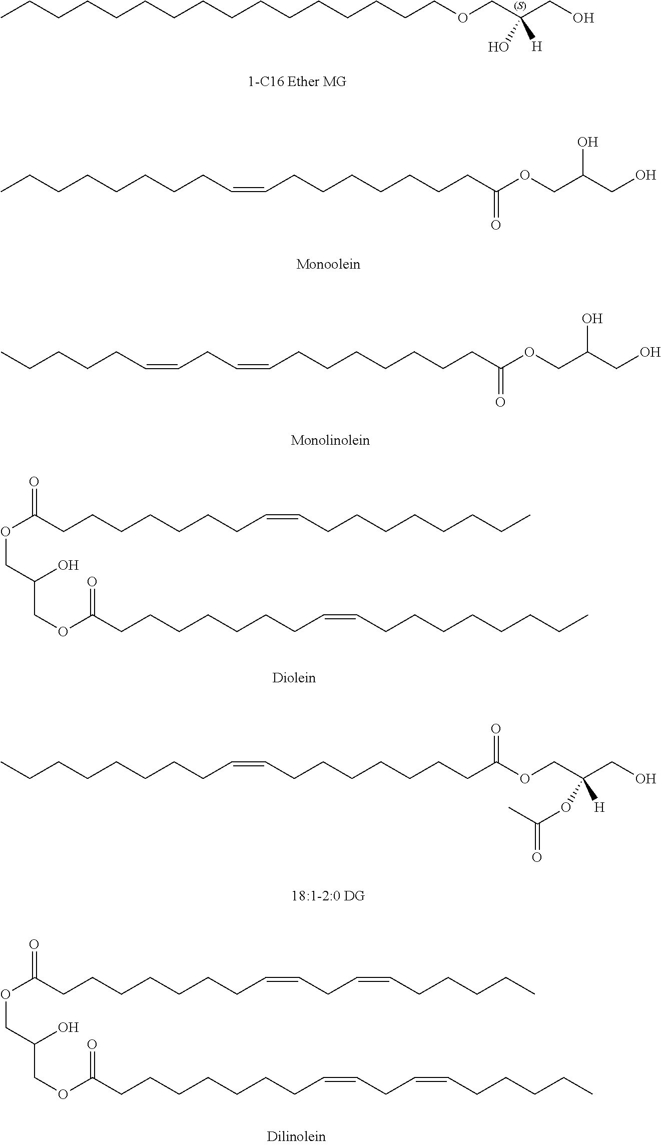

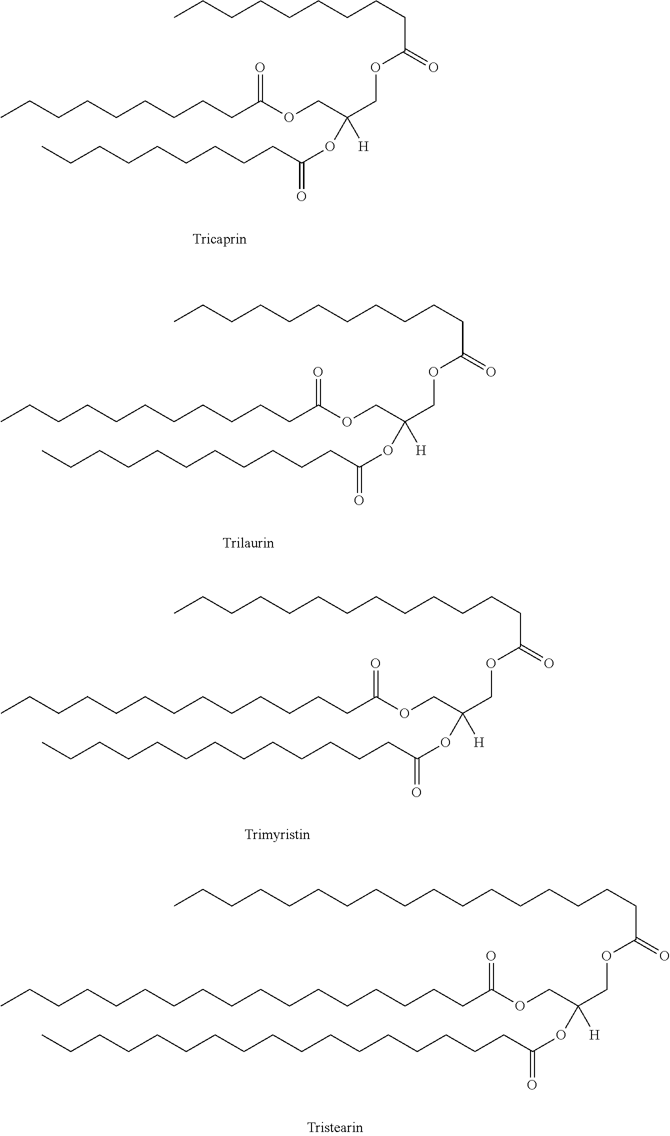 [SMILES: CCCCCCCCCC(=O)OCC(COC(=O)CCCCCCCCC)OC(=O)CCCCCCCCC ; CCCCCCCCCCCC(=O)OCC(COC(=O)CCCCCCCCCCC)OC(=O)CCCCCCCCCCC ; CCCCCCCCCCCCCC(=O)OCC(COC(=O)CCCCCCCCCCCCC)OC(=O)CCCCCCCCCCCCC ; CCCCCCCCCCCCCCCCCC(=O)OCC(COC(=O)CCCCCCCCCCCCCCCCC)OC(=O)CCCCCCCCCCCCCCCCC]

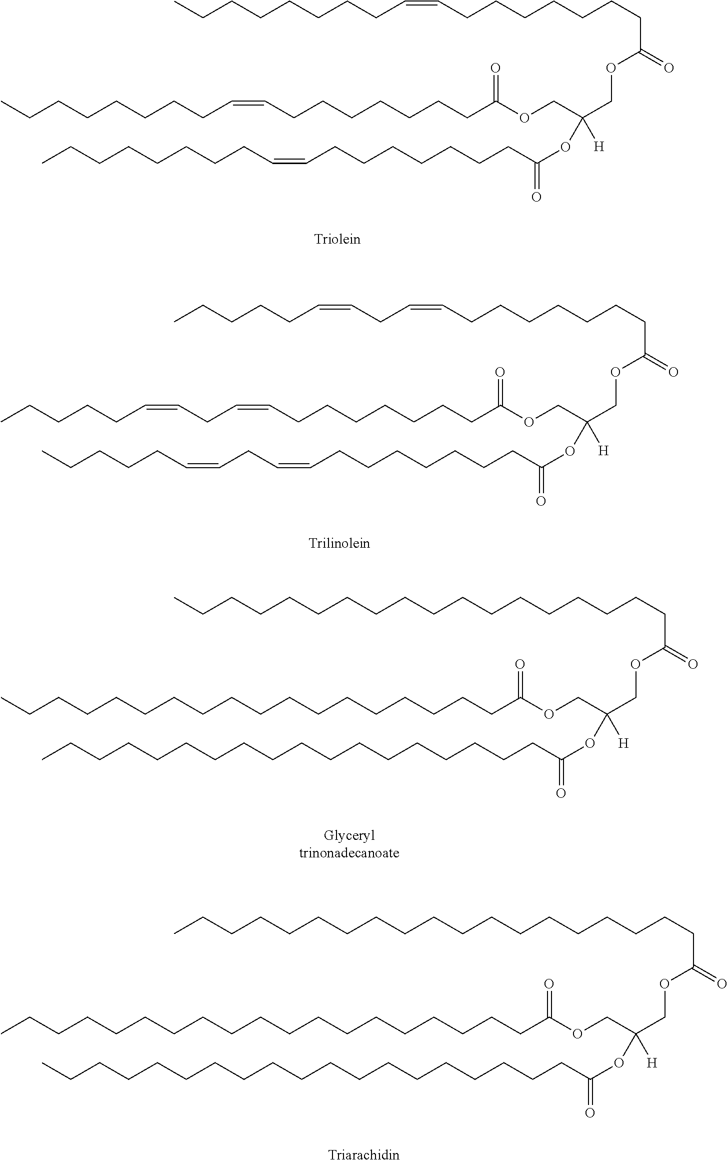 [SMILES: CCCCCCCC/C=C\CCCCCCCC(=O)OCC(COC(=O)CCCCCCC/C=C\CCCCCCCC)OC(=O)CCCCCCC/C=C\CCCCCCCC ; CCCCC/C=C\C/C=C\CCCCCCCC(=O)OCC(COC(=O)CCCCCCC/C=C\C/C=C\CCCCC)OC(=O)CCCCCCC/C=C\C/C=C\CCCCC ; CCCCCCCCCCCCCCCCCCC(=O)OCC(COC(=O)CCCCCCCCCCCCCCCCCC)OC(=O)CCCCCCCCCCCCCCCCCC ; CCCCCCCCCCCCCCCCCCCC(=O)OCC(COC(=O)CCCCCCCCCCCCCCCCCCC)OC(=O)CCCCCCCCCCCCCCCCCCC]

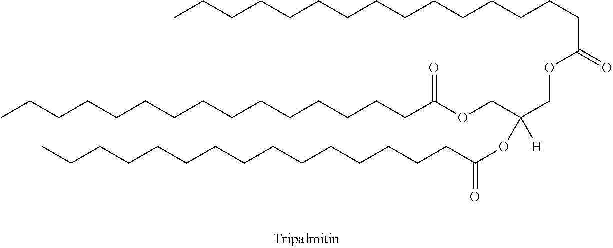 [SMILES: CCCCCCCCCCCCCCCC(=O)OCC(COC(=O)CCCCCCCCCCCCCCC)OC(=O)CCCCCCCCCCCCCCC]

In a 20^{th} embodiment, the glyceride or acylglycol is selected from the group consisting of:

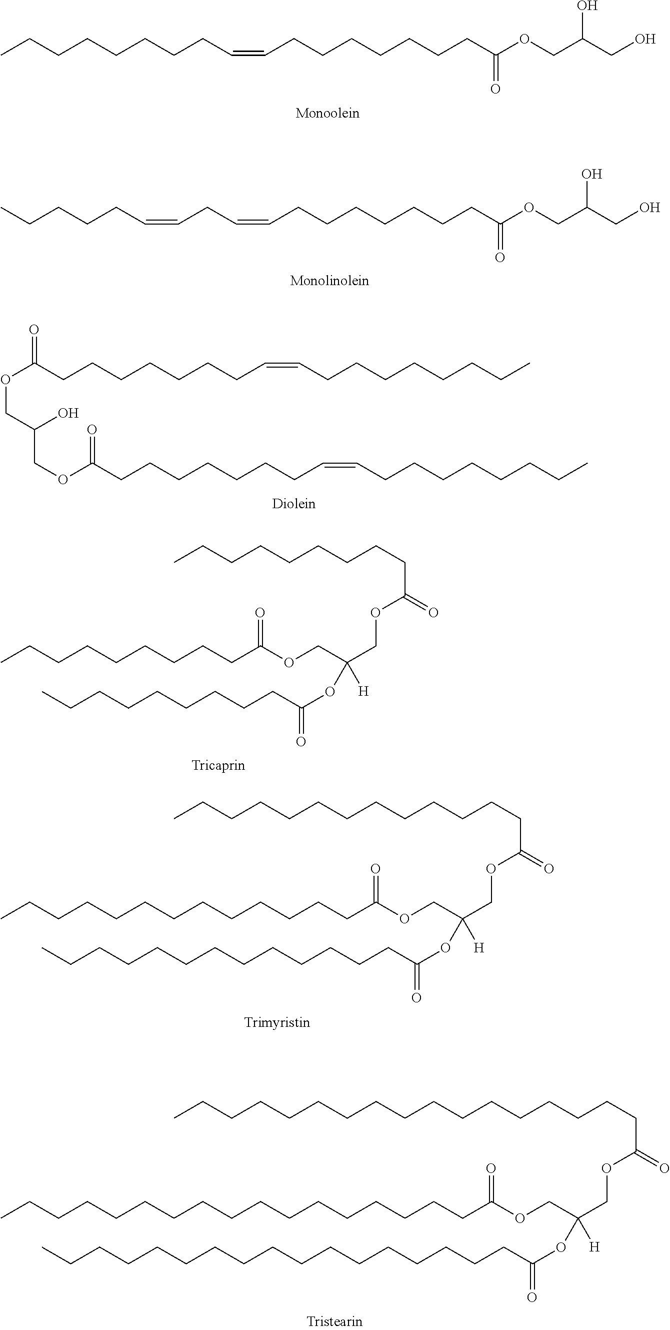

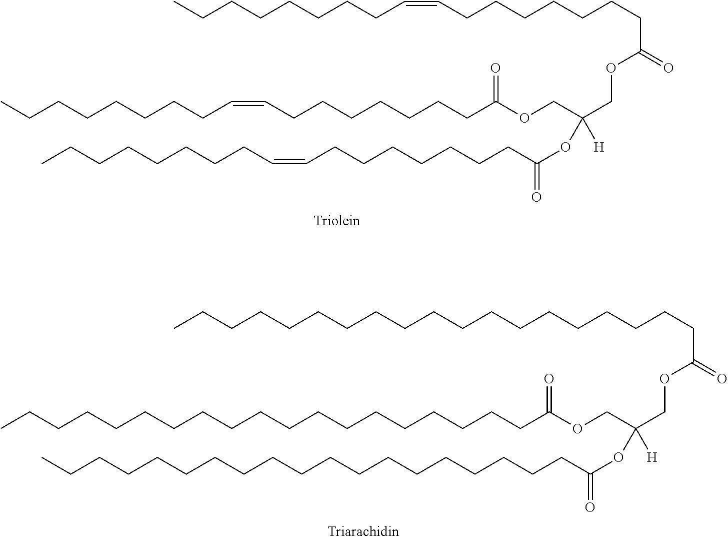 [SMILES: CCCCCCCC/C=C\CCCCCCCC(=O)OCC(COC(=O)CCCCCCC/C=C\CCCCCCCC)OC(=O)CCCCCCC/C=C\CCCCCCCC ; CCCCCCCCCCCCCCCCCCCC(=O)OCC(COC(=O)CCCCCCCCCCCCCCCCCCC)OC(=O)CCCCCCCCCCCCCCCCCCC]

In a 21^{st} embodiment, the glyceride or acylglycol is:

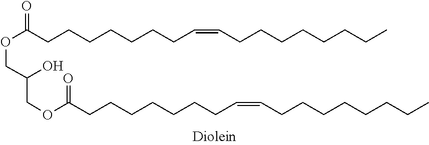 [SMILES: CCCCCCCC/C=C\CCCCCCCC(=O)OCC(O)COC(=O)CCCCCCC/C=C\CCCCCCCC]

In a 22^{nd} embodiment, the glyceride or acylglycol is:

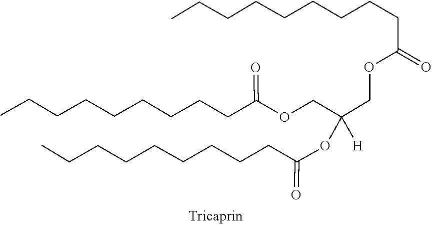 [SMILES: CCCCCCCCCC(=O)OCC(COC(=O)CCCCCCCCC)OC(=O)CCCCCCCCC]

In a 23^{rd} embodiment, the glyceride or acylglycol is:

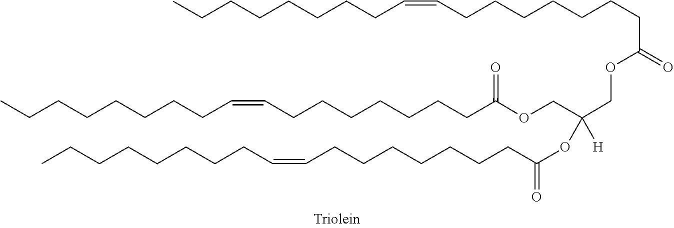 [SMILES: CCCCCCCC/C=C\CCCCCCCC(=O)OCC(COC(=O)CCCCCCC/C=C\CCCCCCCC)OC(=O)CCCCCCC/C=C\CCCCCCCC]

In a 24^{th} embodiment, the ionizable lipid has a structure according to Formula CAT-I:

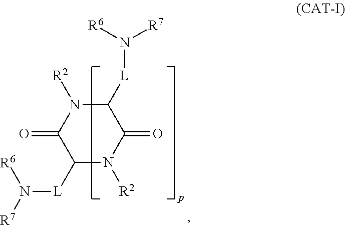

or a pharmaceutically acceptable salt thereof,
wherein:
p is an integer of between 1 and 9, inclusive;
each instance of R² is independently hydrogen or optionally substituted C₁₋₆ alkyl;
each instance of L is independently an optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, or optionally substituted heteroarylene, or combination thereof,
each instance of R⁶ and R⁷ is independently a group of formula (i), (ii), or (iii);
Formulae (i), (ii), and (iii) are:

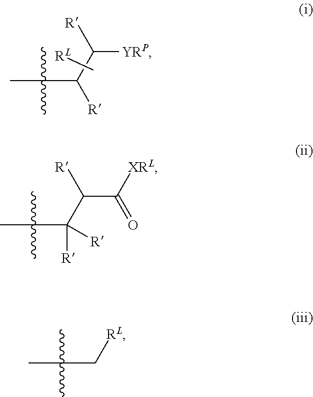

wherein:
each instance of R′ is independently hydrogen or optionally substituted alkyl;
X is O, S, or NR^{X}, wherein R^{X} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
Y is O, S, or NR^{Y}, wherein R^{Y} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
R^{P} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, or a nitrogen protecting group when attached to a nitrogen atom; and
R^{L} is optionally substituted C₁₋₅₀ alkyl, optionally substituted C₂₋₅₀ alkenyl, optionally substituted C₂₋₅₀ alkynyl, optionally substituted heteroC₂₋₅₀ alkyl, optionally substituted heteroC₂₋₅₀ alkenyl, optionally substituted heteroC₂₋₅₀ alkynyl, or a polymer.

In a 25^{th} embodiment, the ionizable lipid has a structure according to Formula CAT-Ia:

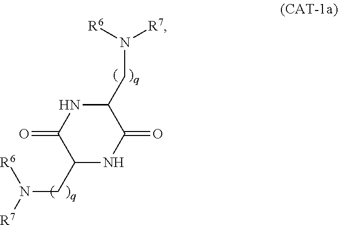

or a pharmaceutically acceptable salt thereof, wherein q is an integer between 1 and 10, inclusive.

In a 26^{th} embodiment, the ionizable lipid has the following structure:

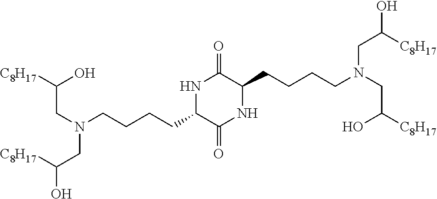

In a 27^{th} embodiment, the ionizable lipid has the following structure:

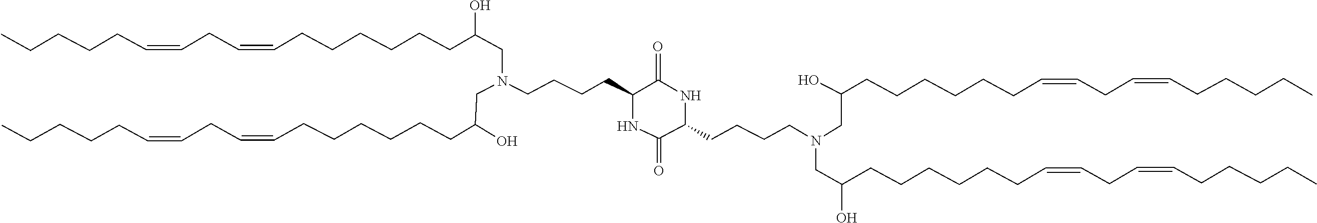 [SMILES: CCCCC/C=C\C/C=C\CCCCCCC(O)CN(CCCC[C@@H]1NC(=O)[C@@H](CCCCN(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC]

In a 28^{th} embodiment, the ionizable lipid has a structure according to Formula CAT-II:

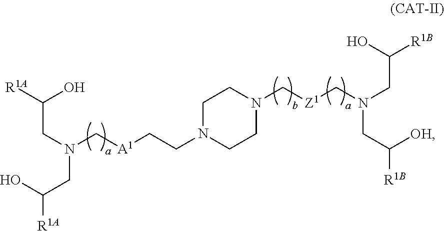

or a pharmaceutically acceptable salt thereof,
wherein:
A¹ is selected from

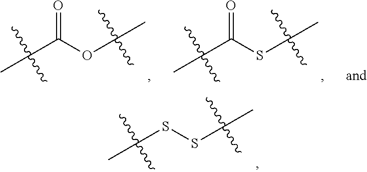

wherein the left hand side of each depicted structure is bound to the —(CH₂)a-;
Z is selected from

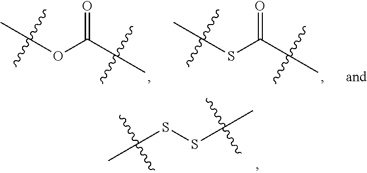

wherein the right hand side of each depicted structure is bound to the —(CH₂)a-;
R^{1A} and R^{1B} are each independently selected from optionally substituted alkyl, optionally substituted alkenyl, optionally substituted alkynyl, optionally substituted acyl, and —W¹—X¹—Y¹;
each W¹ is independently selected from optionally substituted alkyl and optionally substituted alkenyl;
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹,
each Y¹ is independently selected from hydrogen, —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to X¹;
b is 1, 2, 3, 4, or 5; and
   each a is independently selected from 1, 2, 3, 4, 5, 6, 7, 8, 9, and 10.

In a 29^{th} embodiment, the ionizable lipid has a structure according to Formula CAT-IIa:

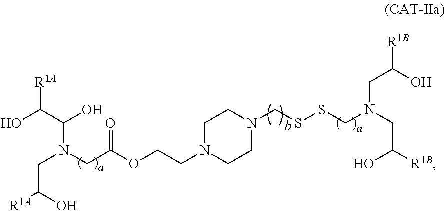

or a pharmaceutically acceptable salt thereof.

In a 30^{th} embodiment, the ionizable lipid has the following structure:

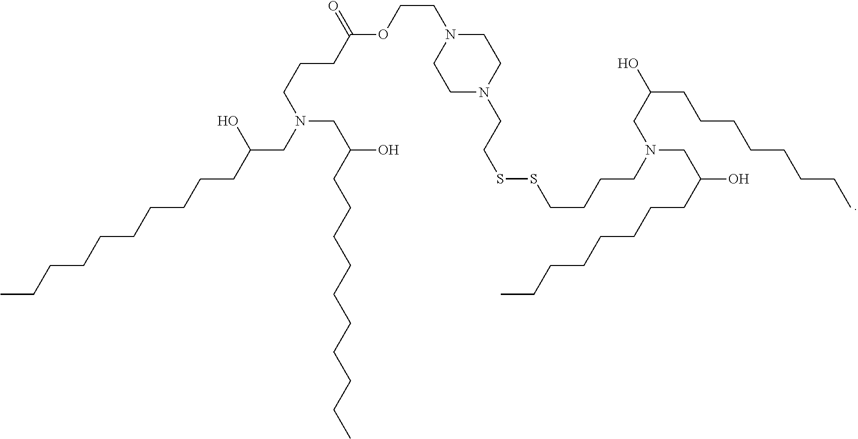 [SMILES: CCCCCCCCCCC(O)CN(CCCC(=O)OCCN1CCN(CCSSCCCCN(CC(O)CCCCCCCC)CC(O)CCCCCCCC)CC1)CC(O)CCCCCCCCCC]

In a 31^{st} embodiment, the ionizable lipid has a structure according to Formula CAT-V:

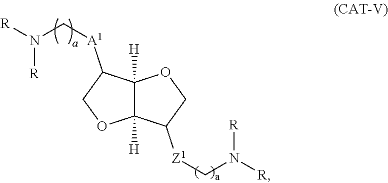

or a pharmaceutically acceptable salt thereof wherein:
A¹ is selected from —C(═O)O—, —C(═O)S—, —C(═O)NH—, —OC(═O)O—, —OC(═O)NH—, —NHC(═O)O—, —SC(═O)NH—, —OCH₂CH₂O—, —OCH₂O—, —OCH(CH₃)O—, —S— and —S—S—, wherein the left hand side of each recited structure is bound to the —(CH₂)ₐ—;
Z¹ is selected from —OC(═O)—, —SC(═O)—, —NHC(═O)—, —OC(═O)O—, —NHC(═O)O—, —OC(═O)NH—, —NHC(═O)S—, —OCH₂CH₂O—, —OCH₂O—, —OCH(CH₃)O—, —S— and —S—S—, wherein the right hand side of each recited structure is bound to the —(CH₂)ₐ—;
each R is independently selected from:

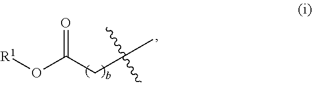

wherein each R¹ is independently selected from optionally substituted alkyl, optionally substituted alkenyl, and optionally substituted alkynyl;

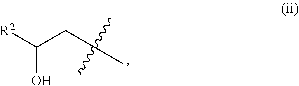

wherein each R² is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;

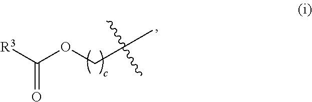

wherein each R³ is independently selected from optionally substituted alkyl, optionally substituted alkenyl, and optionally substituted alkynyl; and

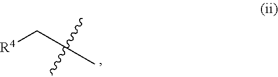

wherein each R⁴ is independently selected from optionally substituted cycloalkyl or optionally substituted heterocycloalkyl;
wherein at least three R are independently selected from (i)

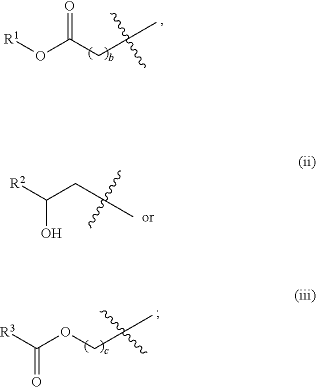

each a is independently selected from 2, 3, 4, and 5;
each b is independently selected from 2, 3, 4, 5, 6, 7, 8, 9 and 10; and
each c is independently selected from 2, 3, 4, 5, 6, 7, 8, 9 and 10.

In a 32^{nd} embodiment, the ionizable lipid has a structure according to Formula CAT-Vb:

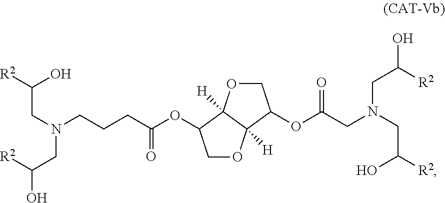

or a pharmaceutically acceptable salt thereof, wherein R² is alkyl.

In a 33^{rd} embodiment, the ionizable lipid has the following structure:

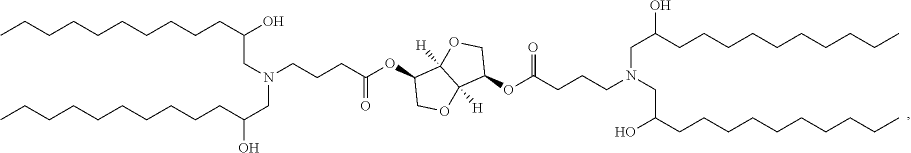 [SMILES: CCCCCCCCCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCCCCCC]

or a pharmaceutically acceptable salt thereof.

In a 34^{th} embodiment, the ionizable lipid has the following structure:

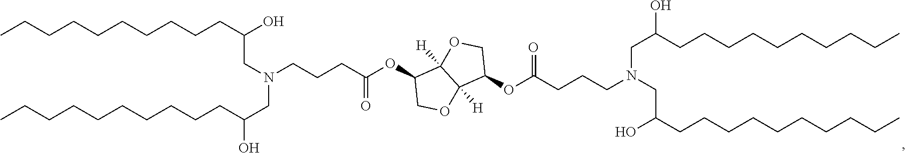 [SMILES: CCCCCCCCCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCCCCCC]

or a pharmaceutically acceptable salt thereof.

In a 35^{th} embodiment, the ionizable lipid has a structure according to Formula CAT-VI:

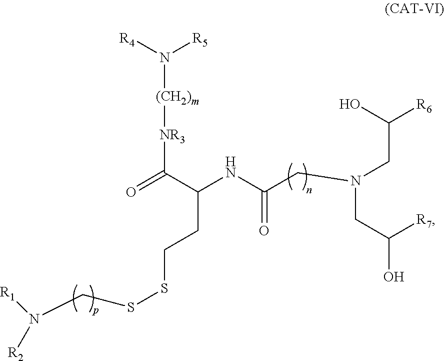

or a pharmaceutically acceptable salt thereof, wherein:
m is an integer chosen between 1 to 6; for example 2 to 4;
n is an integer chosen between 1 to 6; for example 2 to 4;
p is an integer chosen between 1 to 6; for example 2 to 4;
R¹ and R² are independently selected from the group consisting of linear or branched (C₁-C₃₀) alkyl and linear or branched (C₂-C₃₀) alkenyl,
wherein each alkyl and alkenyl are optionally interrupted by one or more groups selected from —C═O—, —C═OO— and —O—, and/or
wherein each alkyl and alkenyl are optionally substituted by one or more substituents selected from —OR, —CN—, —(C₁-C₆) alkyl-OH, —CF₃, —NO₂, —COOR, —SR, halogen atoms and —NRR′;
R³ is selected from the group consisting of H, (C₁-C₆) alkyl optionally substituted by one or more substituents selected from —OR, —CN—, —(C₁-C₆) alkyl-OH, —CF₃, —NO₂, —COOR, —SR, halogen atoms and —NRR′;
R⁴ and R⁵ are independently selected from the group consisting of linear or branched (C₁-C₆) alkyl and linear or branched (C₂-C₆) alkenyl, wherein each alkyl or alkenyl is optionally substituted with one or more of substituents selected from the group consisting of —OR, —CN—, —(C₁-C₆) alkyl-OH, —CF₃, —NO₂, —COOR, —SR, halogen atoms and —NRR′;
or R⁴ and R⁵ together with the N atom to which they are attached form:
a 5 to 6 membered cycloalkyl or heterocycle comprising 1 to 4 heteroatoms selected from O, N and S, or
a 5 to 6 membered aryl or heteroaryl comprising 1 to 4 heteroatoms selected from O, N and S,
wherein said cycloalkyl, heterocycle, aryl or heteroaryl is optionally substituted with one or more substituents selected from —OR, —CN—, —(C₁-C₆) alkyl-OH, —CF₃, —NO₂, —COOR, —SR, halogen atoms and —NRR′;
R⁶ and R⁷ are independently selected from the group consisting of linear or branched (C₁-C₃₀) alkyl and linear or branched (C₂-C₃₀) alkenyl,
wherein each alkyl and alkenyl are optionally interrupted by one or more groups selected from —C═O—, —C═OO— and —O—, and/or
each alkyl and alkenyl are optionally substituted by one or more substituents selected from —OR, —CN—, —C₁-C₆ alkyl-OH, —CF₃, —NO₂, —COOR, —SR, halogen atoms and —NRR′; and
R, R′ are independently selected from H and (C₁-C₆) alkyl.

In a 36^{th} embodiment, the ionizable lipid has the following structure:

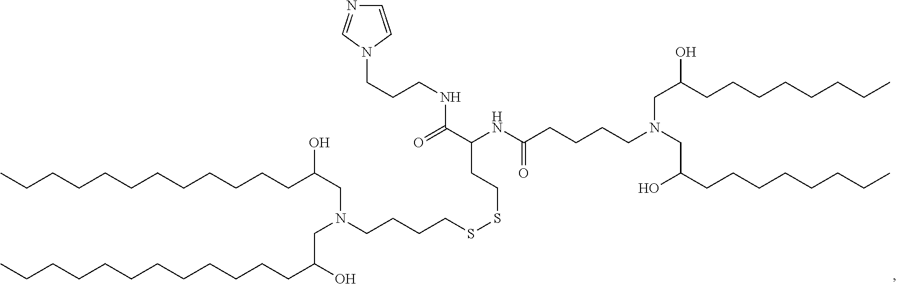 [SMILES: CCCCCCCCCCCCC(O)CN(CCCCSSCCC(NC(=O)CCCCN(CC(O)CCCCCCCC)CC(O)CCCCCCCC)C(=O)NCCCn1ccnc1)CC(O)CCCCCCCCCCCC]

or a pharmaceutically acceptable salt thereof.

In a 37^{th} embodiment, the ionizable lipid is selected from the group consisting of:

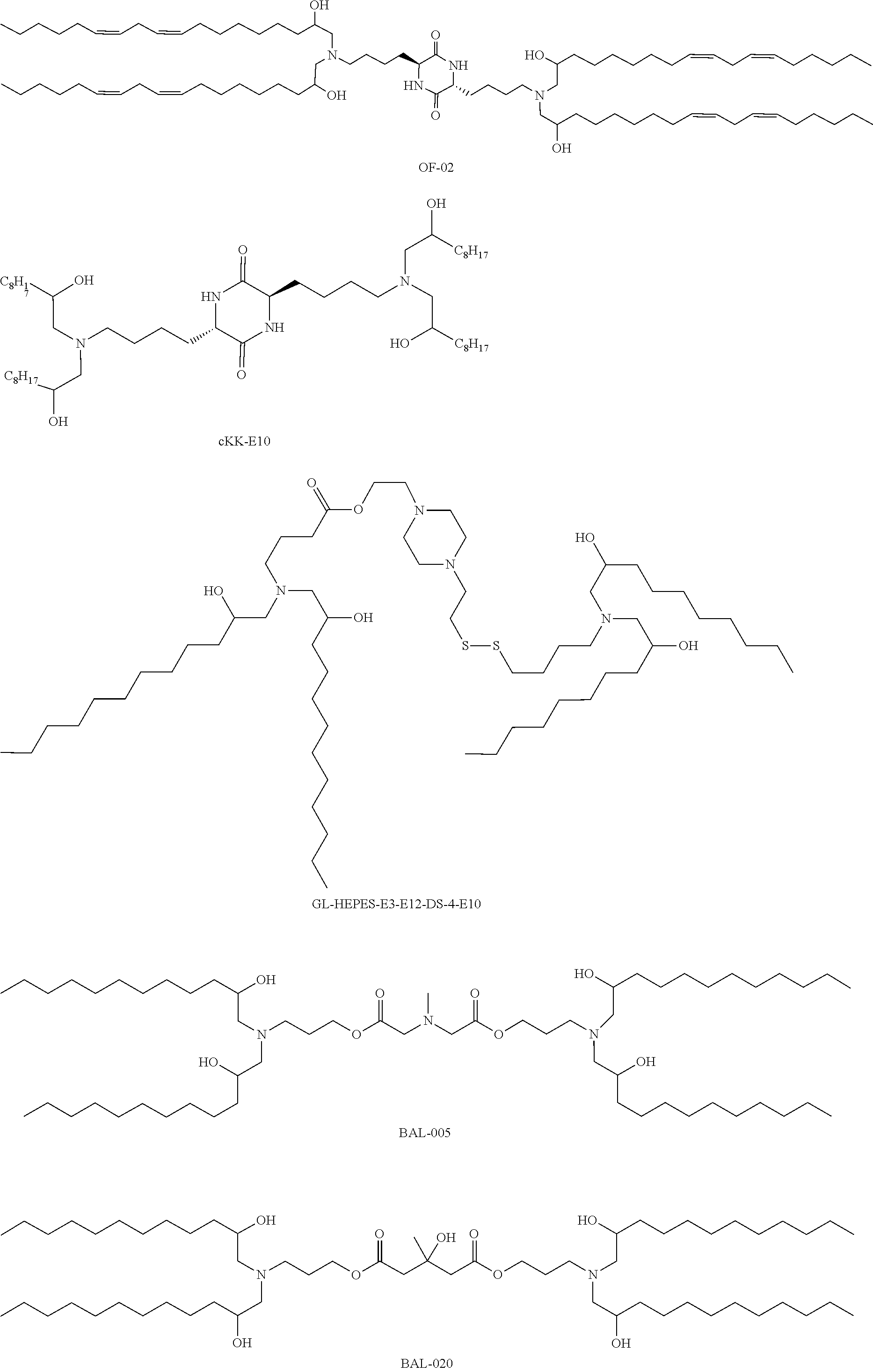

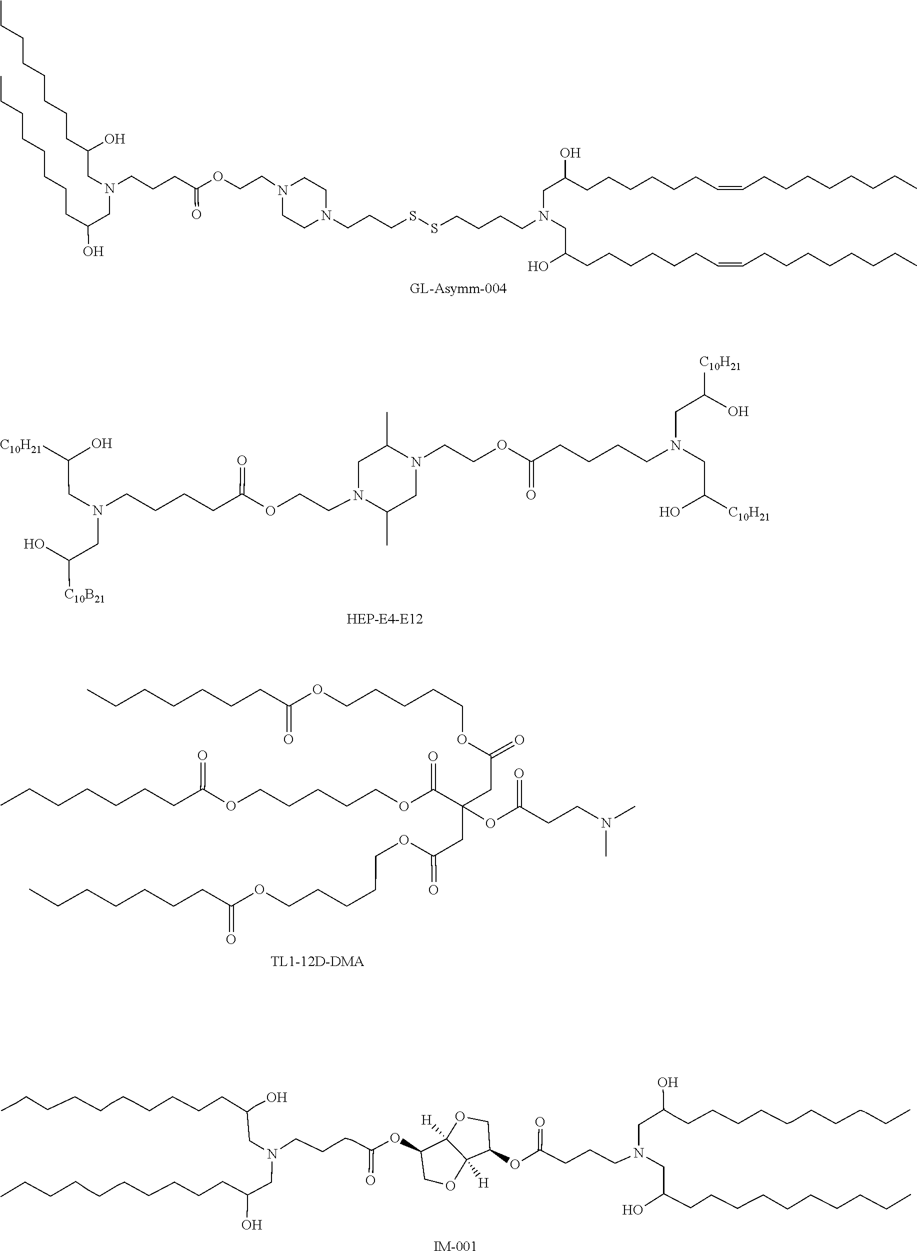

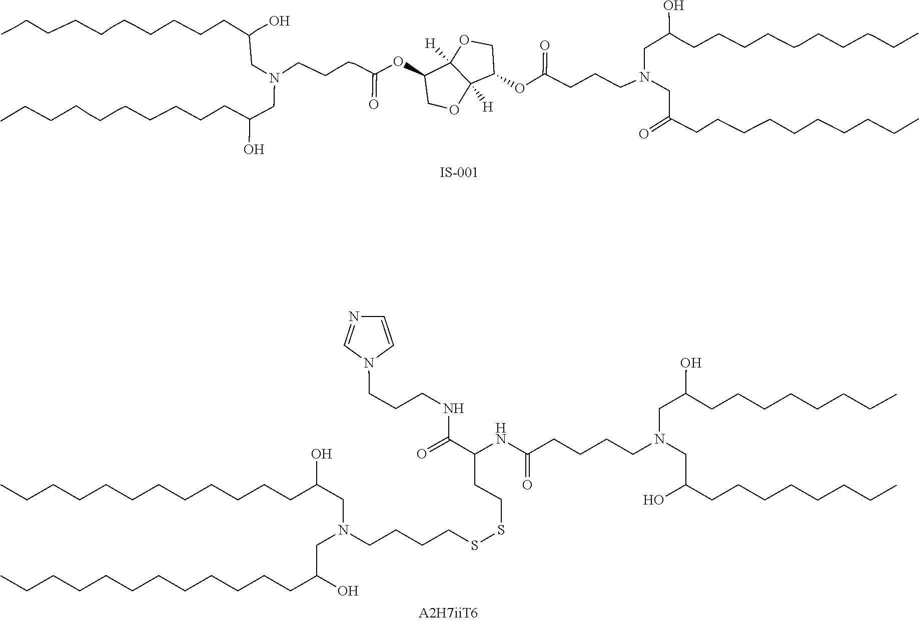

In a 38^{th} embodiment, the ionizable lipid is selected from the group consisting of:

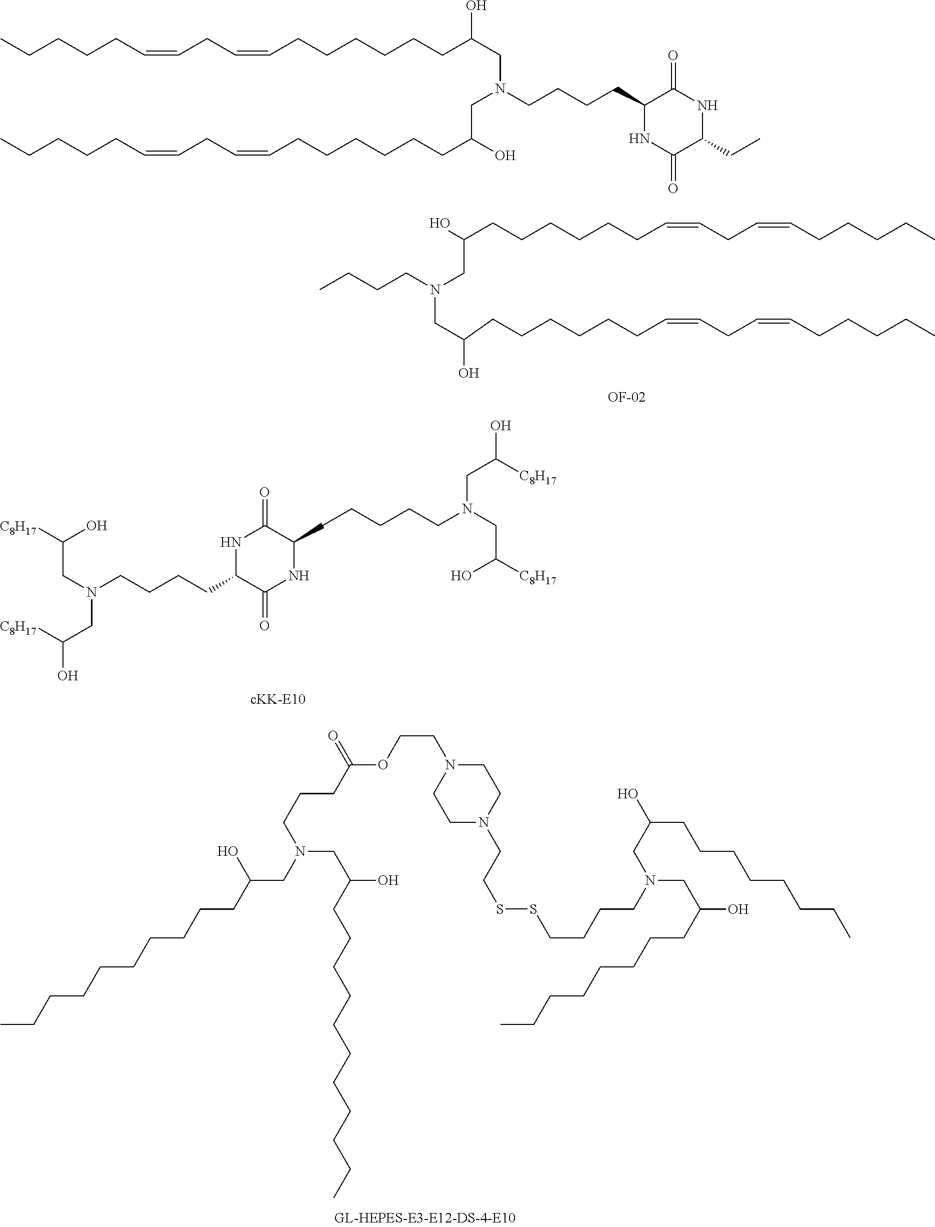

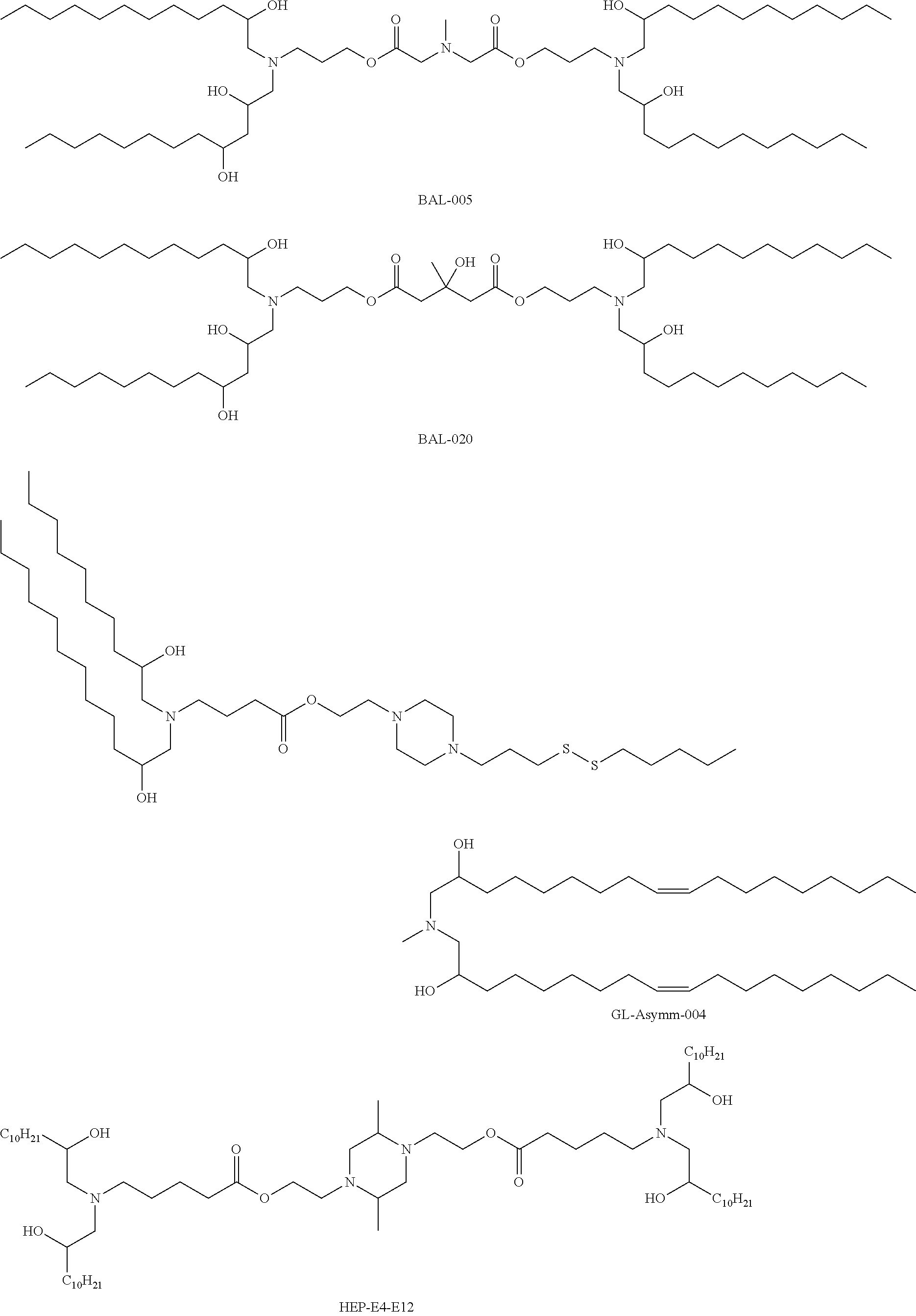

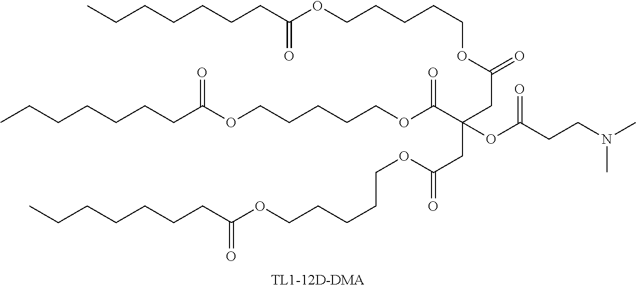 [SMILES: CCCCCCCC(=O)OCCCCCOC(=O)CC(CC(=O)OCCCCCOC(=O)CCCCCCC)(OC(=O)CCN(C)C)C(=O)OCCCCCOC(=O)CCCCCCC]

In a 39^{th} embodiment, the structural lipid is a sterol,

In a 40^{th} embodiment, the structural lipid is a sterol, wherein the sterol is cholesterol.

In a 41^{st} embodiment, the helper lipid is 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), 1,2-dilauroyl-sn-glycero-3-phosphocholine (DLPC), 1,2-Distearoylphosphatidylethanolamine (DSPE), or 1,2-dilauroyl-sn-glycero-3-phosphoethanolamine (DLPE).

In a 42^{nd} embodiment, the helper lipid is 1,2-dioleoyl-SN-glycero-3-phosphoethanolamine (DOPE).

In a 43^{rd} embodiment, the stealth lipid is a polyethylene glycol-conjugated (PEGylated) lipid.

In a 44^{th} embodiment, the stealth lipid is a polyethylene glycol-conjugated (PEGylated) lipid, wherein the PEGylated lipid is 1,2-dimyristoyl-rac-glycero-3-methoxypolyethylene glycol (DMG-PEG), 1,2-distearoyl-sn-glycero-3-phosphoethanolamine-polyethylene glycol (DSPE-PEG), 1,2-dilauroyl-sn-glycero-3-phosphoethanolamine-polyethylene glycol (DLPE-PEG), or 1,2-distearoyl-rac-glycero-polyethelene glycol (DSG-PEG).

In a 45^{th} embodiment, the stealth lipid is a polyethylene glycol-conjugated (PEGylated) lipid, wherein the PEGylated lipid is dimyristoyl-PEG2000 (DMG-PEG).

In a 46^{th} embodiment, the LNP comprises: the ionizable lipid at a molar ratio between 35% and 55%, the structural lipid at a molar ratio between 20% and 35%, the stealth lipid at a molar ratio between 0.25% and 2.75%, and the helper lipid and the glyceride or acylglycol at a combined molar ratio of between 10% and 35%.

In a 47^{th} embodiment, the LNP comprises: the ionizable lipid at a molar ratio of 40%, the structural lipid at a molar ratio 28.5%, the stealth lipid at a molar ratio of 1.5%, and the helper lipid and the glyceride or acylglycol at a combined molar ratio of 30%.

In a 48^{th} embodiment, the LNP comprises: (I) GL-HEPES-E3-E12-DS-4-E10; (II) trimyristin; (III) cholesterol; (IV) DOPE; and (V) DMG-PEG2000.

In a 49^{th} embodiment, the LNP comprises GL-HEPES-E3-E12-DS-4-E10 at a molar ratio between 35% and 45%; cholesterol at a molar ratio between 20% and 35%, DMG-PEG2000 at a molar ratio between 0.25% and 8.75%, DOPE at a molar ratio between 15% and 35%, and trimyristin at a molar ratio between 1% and 10%.

In a 50^{th} embodiment, the LNP comprises GL-HEPES-E3-E12-DS-4-E10 at a molar ratio of about 40%, cholesterol at a molar ratio of about 28.5%, DMG-PEG2000 at a molar ratio of about 1.5%, DOPE at a molar ratio of about 25%, and trimyristin at a molar ratio of about 5%.

In a 51^{st} embodiment, the composition further comprises a nucleic acid molecule, wherein the nucleic acid molecule is encapsulated in the LNP.

In a 52^{nd} embodiment, the LNP comprises 1-20, optionally 5-10 or 6-8, nucleic acid molecules.

In a 53^{rd} embodiment, the nucleic acid molecule is an mRNA molecule.

In a 54^{th} embodiment, the nucleic acid molecule is an mRNA molecule, wherein the mRNA molecule encodes an antigen, optionally a viral antigen or a bacterial antigen.

In a 55^{th} embodiment, the LNP encapsulates two or more mRNA molecules, wherein each mRNA molecule encodes a different antigen, optionally wherein the different antigens are from the same pathogen or from different pathogens.

In a 56^{th} embodiment, the composition comprises two or more LNPs, wherein each LNP encapsulates an mRNA encoding a different antigen, optionally wherein the different antigens are from the same pathogen or from different pathogens.

In a 57^{th} embodiment, the composition is formulated for intramuscular injection.

In a 58^{th} embodiment, the composition comprises a phosphate-buffer saline.

In a 59^{th} embodiment, the composition comprises trehalose, optionally at 10% (w/v) of the composition.

In a 60^{th} embodiment, a method of eliciting an immune response in a subject in need thereof is provided, the method comprising administering to the subject, optionally intramuscularly, intranasally, intravenously, subcutaneously, or intradermally, a prophylactically effective amount of the composition of any of embodiments 51 to 59.

In a 61^{st} embodiment, a method of preventing an infection or reducing one or more symptoms of an infection is provided, the method comprising administering to the subject, optionally intramuscularly, intranasally, intravenously, subcutaneously, or intradermally, a prophylactically effective amount of the composition of any of embodiments 51 to 59.

In a 62^{nd} embodiment, the method of the 60^{th} or 61^{st} embodiment comprises administering to the subject one or more doses of the composition, each dose comprising 1-250, optionally 2.5, 5, 15, 45, or 135, g of mRNA.

In a 63^{rd} embodiment, the method of the 60^{th}, 61^{st}, or 62^{nd} embodiment comprises administering to the subject two doses of the composition with an interval of 2-6, optionally 4, weeks.

In a 64^{th} embodiment, a use of a composition of any of embodiments 51 to 59 for the manufacture of a medicament for use in treating a subject in need thereof, optionally in the method of any of embodiments 60 to 63, is provided.

In a 65^{th} embodiment, the composition of any of embodiments 51 to 59 is provided for use in treating a subject in need thereof, optionally in a method of any of embodiments 60 to 63.

In a 66^{th} embodiment, a kit is provided, wherein the kit comprises a container comprising a single-use or multi-use dosage of the composition of any of embodiments 51 to 59, optionally wherein the container is a vial or a pre-filled syringe or injector.

In order that this invention may be better understood, the following examples are set forth. These examples are for purposes of illustration only and are not to be construed as limiting the scope of the invention in any manner.

### EXAMPLES

The compounds and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

The following examples further illustrate aspects of the present disclosure. However, they are in no way a limitation of the teachings of the present disclosure as set forth.

### Example 1 Formulations

The glycerides described herein can be used in the preparation of lipid nanoparticles according to methods known in the art. For example, suitable methods include methods described in WO 2018/089801, which is hereby incorporated by reference in its entirety.

The lipid nanoparticles in the examples of the present invention were formulated using Process A of WO 2018/089801 (see, e.g., Example 1 and FIG. 1 of WO 2018/089801). Process A (“A”) relates to a conventional method of encapsulating mRNA by mixing mRNA with a mixture of lipids, without first pre-forming the lipids into lipid nanoparticles. In an exemplary process, an ethanolic solution of a mixture of lipids (cationic lipid, phosphatidylethanolamine, cholesterol, and polyethylene glycol-lipid), at a fixed lipid to mRNA ratio, were combined with an aqueous buffered solution of target mRNA at an acidic pH under controlled conditions to yield a suspension of uniform LNPs. After ultrafiltration and diafiltration into a suitable diluent system, the resulting nanoparticle suspensions were diluted to final concentration, filtered, and stored frozen at −80° C. until use.

Lipid nanoparticle formulations were prepared by Process A at the molar ratios lipids disclosed in Table 1 below. The Polydispersity Index (PdI) of lipid nanoparticles can be determined by diluting the formulation in 10% trehalose at about 0.1 mg/ml mRNA concentration and then measuring the size on Malvern zetasizer. The lipid nanoparticle size can be obtained with Malvern Zetasizer Nano-ZS.

Dynamic light scattering (DLS) measurements were performed using a Malvern Instruments Zetasizer with a backscattering detector angle of 173° and a 4-mW, 633-nm He—Ne laser (Worcestershire, UK). The samples were analyzed by diluting in 10% trehalose and measuring the size and Polydispersity Index (PdI) in an optical grade polystyrene cuvette.

With few exceptions, size, PdI and encapsulation efficiency were measured within expected ranges.

TABLE 1
Exemplary LNP Compositions and Characterization Data

| | Cationic | | | | | | | |
|---|---|---|---|---|---|---|---|---|
| Lipid | Lipid | Helper | | | Composition | Size | | EE |
| Family | (Cat) | Lipid | PEG | Glyceride | (PEG:cat:chol:help:add) | (nm) | PdI | (%) |
| Bis AIM | BAL- | DOPE | DMG- | Triolein | 1.5:40:28.5:25:5 | 113 | 0.029 | 47 |
| lipid | 005 | | PEG- | | | | | |
| | | | 2000 | | | | | |
| Bis AIM | BAL- | DOPE | DMG- | Tricaprin | 1.5:40:28.5:25:5 | 106 | 0.104 | 55 |
| lipid | 005 | | PEG- | | | | | |
| | | | 2000 | | | | | |
| Bis AIM | BAL- | DOPE | DMG- | Triolein | 1.5:40:28.5:25:5 | 98 | 0.109 | 91 |
| | 020 | | PEG- | | | | | |
| | | | 2000 | | | | | |
| Bis AIM | BAL- | DOPE | DMG- | Tricaprin | 1.5:40:28.5:25:5 | 98 | 0.061 | 92 |
| | 020 | | PEG- | | | | | |
| | | | 2000 | | | | | |
| cKK | cKK- | DOPE | DMG- | Triolein | 1.5:40:28.5:21.53:8.47 | 97 | 0.091 | 74 |
| | E10 | | PEG- | | | | | |
| | | | 2000 | | | | | |
| cKK | cKK- | DOPE | DMG- | Tricaprin | 1.5:40:28.5:21.53:8.47 | 105 | 0.106 | 88 |
| | E10 | | PEG- | | | | | |
| | | | 2000 | | | | | |
| cKK | cKK- | DOPE | DMG- | Triolein | 1.5:40:28.5:28.94:1.06 | 97 | 0.142 | 90 |
| | E10 | | PEG- | | | | | |
| | | | 2000 | | | | | |
| cKK | cKK- | DOPE | DMG- | Tricaprin | 1.5:40:28.5:28.94:1.06 | 104 | 0.106 | 85 |
| | E10 | | PEG- | | | | | |
| | | | 2000 | | | | | |
| cKK | cKK- | DOPE | DMG- | Triolein | 1.5:40:28.5:28.93:1.07 | 94 | 0.115 | 91 |
| | E10 | | PEG- | | | | | |
| | | | 2000 | | | | | |
| cKK | cKK- | DOPE | DMG- | Tricaprin | 1.5:40:28.5:28.93:1.07 | 108 | 0.090 | 82 |
| | E10 | | PEG- | | | | | |
| | | | 2000 | | | | | |
| cKK | cKK- | DOPE | DMG- | Triolein | 1.5:40:28.5:25:5 | 109 | 0.058 | 78 |
| | E10 | | PEG- | | | | | |
| | | | 2000 | | | | | |
| cKK | cKK- | DOPE | DMG- | Triolein | 1.5:40:28.5:20.29:9.71 | 95 | 0.087 | 84 |
| | E10 | | PEG- | | | | | |
| | | | 2000 | | | | | |
| cKK | cKK- | DOPE | DMG- | Tricaprin | 1.5:40:28.5:20.29:9.71 | 101 | 0.104 | 87 |
| | E10 | | PEG- | | | | | |
| | | | 2000 | | | | | |
| cKK | cKK- | DOPE | DMG- | Monolinolein | 1.5:40:28.5:25:5 | 112 | 0.152 | 85 |
| | E10 | | PEG- | | | | | |
| | | | 2000 | | | | | |
| cKK | cKK- | DOPE | DMG- | Triolein | 1.5:40:28.5:22.43:7.57 | 93 | 0.09 | 87 |
| | E10 | | PEG- | | | | | |
| | | | 2000 | | | | | |
| cKK | cKK- | DOPE | DMG- | Tricaprin | 1.5:40:28.5:25:5 | 114 | 0.067 | 83 |
| | E10 | | PEG- | | | | | |
| | | | 2000 | | | | | |
| cKK | cKK- | DOPE | DMG- | Tricaprin | 1.5:40:28.5:22.43:7.57 | 103 | 0.124 | 88 |
| | E10 | | PEG- | | | | | |
| | | | 2000 | | | | | |
| cKK | cKK- | DOPE | DMG- | Dilinolein | 1.5:40:28.5:25:5 | 111 | 0.144 | 78 |
| | E10 | | PEG- | | | | | |
| | | | 2000 | | | | | |
| cKK | cKK- | DOPE | DMG- | Triolein | 1.5:40:28.5:25:5 | 113 | 0.126 | 83 |
| | E10 | | PEG- | | | | | |
| | | | 2000 | | | | | |
| cKK | cKK. | DOPE | DMG- | Triolein | 1.5:40:28.5:28.83:1.17 | 96 | 0.099 | 89 |
| | E10 | | PEG- | | | | | |
| | | | 2000 | | | | | |
| cKK | cKK- | DOPE | DMG- | Tricaprin | 1.5:40:28.5:28.83:1.17 | 108 | 0.096 | 81 |
| | E10 | | PEG- | | | | | |
| | | | 2000 | | | | | |
| cKK | cKK- | DOPE | DMG- | Monoolein | 1.5:40:28.5:25:5 | 110 | 0.109 | 84 |
| | E10 | | PEG- | | | | | |
| | | | 2000 | | | | | |
| cKK | cKK- | DOPE | DMG- | Tristearin | 1.5:40:28.5:25:5 | 114 | 0.149 | 86 |
| | E10 | | PEG- | | | | | |
| | | | 2000 | | | | | |
| cKK | cKK- | DOPE | DMG- | Triolein | 1.5:40:28.5:26.69:3.31 | 91 | 0.119 | 89 |
| | E10 | | PEG- | | | | | |
| | | | 2000 | | | | | |
| cKK | cKK- | DOPE | DMG- | Tricaprin | 1.5:40:28.5:26.69:3.31 | 108 | 0.114 | 82 |
| | E10 | | PEG- | | | | | |
| | | | 2000 | | | | | |
| cKK | cKK- | DOPE | DMG- | Diolein | 1.5:40:28.5:25:5 | 108 | 0.112 | 85 |
| | E10 | | PEG- | | | | | |
| | | | 2000 | | | | | |
| cKK | cKK- | DOPE | DMG- | Triolein | 1.5:40:28.5:22.48:7.52 | 98 | 0.102 | 58 |
| | E10 | | PEG- | | | | | |
| | | | 2000 | | | | | |
| cKK | cKK- | DOPE | DMG- | Trimyristin | 1.5:40:28.5:25:5 | 114 | 0.127 | 86 |
| | E10 | | PEG- | | | | | |
| | | | 2000 | | | | | |
| cKK | cKK- | DOPE | DMG- | Trilinolein | 1.5:40:28.5:25:5 | 103 | 0.115 | 85 |
| | E10 | | PEG- | | | | | |
| | | | 2000 | | | | | |
| cKK | cKK- | DOPE | DMG- | Tricaprin | 1.5:40:28.5:22.48:7.52 | 104 | 0.103 | 86 |
| | E10 | | PEG- | | | | | |
| | | | 2000 | | | | | |
| cKK | cKK- | DOPE | DMG- | Tricaprin | 1.5:40:28.5:25:5 | 108 | 0.112 | 85 |
| | E10 | | PEG- | | | | | |
| | | | 2000 | | | | | |
| cKK | cKK- | DOPE | DMG- | Triarachidin | 1.5:40:28.5:25:5 | 123 | 0.050 | 86 |
| | E10 | | PEG- | | | | | |
| | | | 2000 | | | | | |
| GOOD | GL- | DOPE | DMG- | Triolein | 1.5:40:28.5:25:5 | 98 | 0.097 | 95 |
| Asymmetric | Asymm- | | PEG- | | | | | |
| | 004 | | 2000 | | | | | |
| GOOD | GL- | DOPE | DMG- | Tricaprin | 1.5:40:28.5:25:5 | 94 | 0.089 | 95 |
| Asymmetric | Asymm- | | PEG- | | | | | |
| | 004 | | 2000 | | | | | |
| GOOD | GL- | DOPE | DMG- | 10:0 DG | 1.5:40:28.5:25:5 | 92 | 0.165 | 66 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | 08:0 DG | 1.5:40:28.5:25:5 | 93 | 0.138 | 60 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | 18:1 Ethylene | 1.5:40:28.5:25:5 | 109 | 0.214 | 67 |
| HEPES | HEPES- | | PEG- | Glycol | | | | |
| | E3-E12- | | 2000 | | | | | |
| | DS-4- | | | | | | | |
| | E10 | | | | | | | |
| GOOD | GL- | DOPE | DMG- | 16:0 Ethylene | 1.5:40:28.5:25:5 | 107 | 0.195 | 71 |
| HEPES | HEPES- | | PEG- | Glycol | | | | |
| | E3-E12- | | 2000 | | | | | |
| | DS-4- | | | | | | | |
| | E10 | | | | | | | |
| GOOD | GL- | DOPE | DMG- | 18:1-2:0 DG | 1.5:40:28.5:25:5 | 103 | 0.192 | 81 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | 1-C16 Ether MG | 1.5:40:28.5:25:5 | 84 | 0.132 | 83 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | 16:0-(12- | 1.5:40:28.5:25:5 | 113 | 0.071 | 61 |
| HEPES | HEPES- | | PEG- | PAHSA)-18:1 | | | | |
| | E3-E12- | | 2000 | TG | | | | |
| | DS-4- | | | | | | | |
| | E10 | | | | | | | |
| GOOD | GL- | DOPE | DMG- | C18(plasm) MG | 1.5:40:28.5:25:5 | 87 | 0.159 | 70 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Monolinolein | 1.5:40:28.5:25:5 | 91 | 0.121 | 86 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Monolinolein | 1.5:40:28.5:25:5 | 91 | 0.121 | 86 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Tripalmitin | 1.5:40:28.5:25:5 | 113 | 0.143 | 97 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Monoolein | 1.5:40:28.5:25:5 | 87 | 0.116 | 86 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Monoolein | 1.5:40:28.5:25:5 | 87 | 0.116 | 86 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Dilinolein | 1.5:40:28.5:25:5 | 84 | 0.132 | 87 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Dilinolein | 1.5:40:28.5:25:5 | 84 | 0.132 | 87 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Diolein | 1.5:40:28.5:25:5 | 84 | 0.126 | 88 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Diolein | 1.5:40:28.5:25:5 | 84 | 0.126 | 88 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Trilinolein | 1.5:40:28.5:25:5 | 87 | 0.159 | 89 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Trilinolein | 1.5:40:28.5:25:5 | 87 | 0.159 | 89 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Trimyristin | 1.5:40:28.5:25:5 | 88 | 0.125 | 90 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Trimyristin | 1.5:40:28.5:25:5 | 88 | 0.125 | 90 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Tricaprin | 1.5:40:28.5:25:5 | 91 | 0.115 | 93 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Tristearin | 1.5:40:28.5:25:5 | 97 | 0.109 | 90 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Tristearin | 1.5:40:28.5:25:5 | 97 | 0.109 | 90 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Triolein | 1.5:40:28.5:25:5 | 91 | 0.107 | 93 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Triarachidin | 1.5:40:28.5:25:5 | 100 | 0.161 | 90 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | Triarachidin | 1.5:40:28.5:25:5 | 100 | 0.161 | 90 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | 15:0-18:1 DG | 1.5:40:28.5:25:5 | 85 | 0.429 | 59 |
| HEPES | HEPES- | | PEG- | | | | | |
| | E3-E12- | | 2000 | | | | | |
| | DS-4- | | | | | | | |
| | E10 | | | | | | | |
| GOOD | GL- | DOPE | DMG- | 15:0-18:1-15:0 | 1.5:40:28.5:25:5 | 157 | 0.321 | 37 |
| HEPES | HEPES- | | PEG- | TG | | | | |
| | E3-E12- | | 2000 | | | | | |
| | DS-4- | | | | | | | |
| | E10 | | | | | | | |
| Piperazine | HEP- | DOPE | DMG- | Tricaprin | 1.5:40:28.5:25:5 | 92 | 0.124 | 76 |
| | E4-E12 | | PEG- | | | | | |
| | | | 2000 | | | | | |
| Piperazine | HEP- | DOPE | DMG- | Triolein | 1.5:40:28.5:25:5 | 94 | 0.115 | 83 |
| | E4-E12 | | PEG- | | | | | |
| | | | 2000 | | | | | |
| cKK | OF-02 | DOPE | DMG- | Tricaproin | 1.5:40:28.5:25:5 | 118 | 0.079 | 89 |
| | | | PEG- | | | | | |
| | | | 2000 | | | | | |
| cKK | OF-02 | DOPE | DMG- | Trilaurin | 1.5:40:28.5:25:5 | 116 | 0.101 | 88 |
| | | | PEG- | | | | | |
| | | | 2000 | | | | | |
| cKK | OF-02 | DOPE | DMG- | Monolinolein | 1.5:40:28.5:25:5 | 126 | 0.082 | 90 |
| | | | PEG- | | | | | |
| | | | 2000 | | | | | |
| cKK | OF-02 | DOPE | DMG- | Tributyrin | 1.5:40:28.5:25:5 | 120 | 0.107 | 88 |
| | | | PEG- | | | | | |
| | | | 2000 | | | | | |
| cKK | OF-02 | DOPE | DMG- | Monoolein | 1.5:40:28.5:25:5 | 121 | 0.103 | 85 |
| | | | PEG- | | | | | |
| | | | 2000 | | | | | |
| cKK | OF-02 | DOPE | DMG- | Tricaprin | 1.5:40:28.5:25:5 | 107 | 0.083 | 90 |
| | | | PEG- | | | | | |
| | | | 2000 | | | | | |
| cKK | OF-02 | DOPE | DMG- | Glyceryl | 1.5:40:28.5:25:5 | 142 | 0.058 | 87 |
| | | | PEG- | trinonadecanoate | | | | |
| | | | 2000 | | | | | |
| cKK | OF-02 | DOPE | DMG- | Dilinolein | 1.5:40:28.5:25:5 | 124 | 0.083 | 86 |
| | | | PEG- | | | | | |
| | | | 2000 | | | | | |
| cKK | OF-02 | DOPE | DMG- | Triolein | 1.5:40:28.5:25:5 | 111 | 0.08 | 100 |
| | | | PEG- | | | | | |
| | | | 2000 | | | | | |
| cKK | OF-02 | DOPE | DMG- | Diolein | 1.5:40:28.5:25:5 | 130 | 0.118 | 85 |
| | | | PEG- | | | | | |
| | | | 2000 | | | | | |
| cKK | OF-02 | DOPE | DMG- | Trilinolein | 1.5:40:28.5:25:5 | 127 | 0.085 | 86 |
| | | | PEG- | | | | | |
| | | | 2000 | | | | | |
| cKK | OF-02 | DOPE | DMG- | Trimyristin | 1.5:40:28.5:25:5 | 121 | 0.064 | 87 |
| | | | PEG- | | | | | |
| | | | 2000 | | | | | |
| cKK | OF-02 | DOPE | DMG- | Tristearin | 1.5:40:28.5:25:5 | 126 | 0.083 | 86 |
| | | | PEG- | | | | | |
| | | | 2000 | | | | | |
| cKK | OF-02 | DOPE | DMG- | Triarachidin | 1.5:40:28.5:25:5 | 142 | 0.071 | 86 |
| | | | PEG- | | | | | |
| | | | 2000 | | | | | |
| Trident | TL1- | DOPE | DMG- | Monolinolein | 1.5:40:28.5:25:5 | 91 | 0.134 | 92 |
| | 12D- | | PEG- | | | | | |
| | DMA | | 2000 | | | | | |
| Trident | TL1- | DOPE | DMG- | Dilinolein | 1.5:40:28.5:25:5 | 94 | 0.151 | 92 |
| | 12D- | | PEG- | | | | | |
| | DMA | | 2000 | | | | | |
| Trident | TL1- | DOPE | DMG- | Triolein | 1.5:40:28.5:25:5 | 98 | 0.126 | 93 |
| | 12D- | | PEG- | | | | | |
| | DMA | | 2000 | | | | | |
| Trident | TL1- | DOPE | DMG- | Tristearin | 1.5:40:28.5:25:5 | 99 | 0.135 | 92 |
| | 12D- | | PEG- | | | | | |
| | DMA | | 2000 | | | | | |
| Trident | TL1- | DOPE | DMG- | Trimyristin | 1.5:40:28.5:25:5 | 84 | 0.144 | 92 |
| | 12D- | | PEG- | | | | | |
| | DMA | | 2000 | | | | | |
| Trident | TL1- | DOPE | DMG- | Tricaprin | 1.5:40:28.5:25:5 | 93 | 0.139 | 93 |
| | 12D- | | PEG- | | | | | |
| | DMA | | 2000 | | | | | |

### Example 2—IM EPO Data

To determine the activity of LNPs containing glycerides, EPO expression studies were conducted with female BALB/cJ mice 6-8 weeks of age (n=4 per group). Mice were dosed with 0.1 μg in 30 μL of LNPs by a single intramuscular (IM) injection into the gastrocnemius leg muscle. Blood samples were taken 6- and 24-hours post injection and hEPO levels were measured in the blood serum of the mice using an ELISA assay according to the manufacture's protocol. All LNPs were provided at 1.5:40:28.5:25:5 (PEG:cat:chol:help:glyceride), where PEG is DMG-PEG-2000, cat is the cationic lipid, and help is DOPE WO2022/099003 A1 also describes an in vivo assay for intramuscular administration (e.g. on page 46, paragraph [00206]). The data for various cationic lipid compositions, e.g., are presented in the Tables 2-7, normalized for each to the LNP formulation without addition of glycerides.

In general, the addition of different glycerides provided an increase in EPO expression using various ionizable lipids.

TABLE 2
EPO Expression of OF-02 LNPs

| Glyceride | Mean | SD |
|---|---|---|
| OF-02 control / no glyceride | 1 | 0.4 |
| Tristearin | 0.43 | 0.19 |
| Trimyristin | 0.52 | 0.29 |
| Monolinolein | 0.52 | 0.27 |
| Monoolein | 0.53 | 0.28 |
| Diolein | 0.53 | 0.38 |
| Triarachidin | 0.56 | 0.29 |
| Trilinolein | 0.61 | 0.26 |
| Tributyrin | 0.67 | 0.38 |
| Glyceryl trinonadecanoate | 0.8 | 0.49 |
| Dilinolein | 0.8 | 0.4 |
| Tricaproin | 0.87 | 0.69 |
| Triolein | 0.91 | 0.26 |
| Trilaurin | 0.98 | 0.52 |
| Tricaprin | 1.79 | 0.58 |

TABLE 3
EPO Expression of cKK-E10 LNPs

| Glyceride | Mean | SD |
|---|---|---|
| cKK-E10 Control / no glyceride | 1 | 0.3 |
| Triarachidin | 0.79 | 0.33 |
| Monolinolein | 0.8 | 0.23 |
| Dilinolein | 0.83 | 0.23 |
| Diolein | 1.11 | 0.5 |
| Monoolein | 1.12 | 0.36 |
| Tristearin | 1.2 | 0.32 |
| Trilinolein | 1.21 | 0.63 |
| Triolein | 1.22 | 0.36 |
| Trimyristin | 1.22 | 0.29 |
| Tricaprin | 1.54 | 0.52 |

TABLE 4
EPO Expression of GL-HEPES-E3-E12-DS-4-E10 LNPs

| Glyceride | Mean | SD |
|---|---|---|
| GL-HEPES-E3-E12-DS-4-E10 control / no | 1 | 0.3 |
| glyceride | | |
| Trilinolein | 1.6 | 0.5 |
| Monolinolein | 1.7 | 0.5 |
| Dilinolein | 1.9 | 0.4 |
| Trimyristin | 2.2 | 0.7 |
| Monoolein | 2.3 | 0.6 |
| Triarachidin | 2.5 | 0.8 |
| Tristearin | 3.1 | 0.8 |
| Diolein | 3.8 | 0.8 |

TABLE 5
EPO Expression of TL1-12D-DMA LNPs

| Glyceride | Mean | SD |
|---|---|---|
| TL1-12D-DMA Control / no glyceride | 1 | 0.2 |
| Tricaprin | 1.2 | 0.4 |
| Trimyristin | 1 | 0.2 |
| Tristearin | 0.9 | 0.3 |
| Triolein | 1.4 | 0.6 |
| Dilinolein | 0.7 | 0.4 |
| Monolinolein | 0.6 | 0.3 |

TABLE 6
EPO Expression of HEP-E4-E12 LNPs

| Glyceride | Mean | SD |
|---|---|---|
| HEP-E4-E12 Control / no glyceride | 1 | 0.1 |
| Tricaprin | 0.6 | 0.1 |
| Triolein | 0.8 | 0.2 |

TABLE 7
EPO Expression of BAL-005 LNPs

| Glyceride | Mean | SD |
|---|---|---|
| BAL-005 Control / no glyceride | 1 | 0.1 |
| Tricaprin | 0.7 | 0.2 |
| Triolein | 0.8 | 0.1 |

### Example 3—Modification of Glyceride % in Compositions

Modification of the glyceride percentages in compositions was also investigated. LNPs containing cKK-E10 were produced with tricaprin or triolein from 0 to 9.71%. EPO expression studies were conducted with female BALB/cJ mice 6-8 weeks of age (n=4 per group). Mice were dosed with 0.1 μg in 30 μL of LNPs by a single intramuscular (IM) injection into the gastrocnemius leg muscle. Blood samples were taken 6- and 24-hours post injection and hEPO levels were measured in the blood serum of the mice using an ELISA assay according to the manufacture's protocol. The data for various LNP compositions are provided in Tables 8 and 9, below, normalized to control compositions without addition of glycerides.

Overall, the EPO expression was increased using glycerides at majority of the molar % ratios of glycerides tested.

TABLE 8
EPO Expression of cKK-E10 LNPs with Tricaprin

| LNP Formulation (DMG-PEG2k:cKK- | | | |
|---|---|---|---|
| E10:Chol:DOPE:Tricaprin) | Mean | SD | N |
| 1.5:40:28.5:30:0 | 1 | 0.3 | 4 |
| 1.5:40:28.5:28.94:1.06 | 1.8 | 0.4 | 4 |
| 1.5:40:28.5:28.93:1.07 | 1.3 | 0.4 | 4 |
| 1.5:40:28.5:28.83:1.17 | 1.6 | 0.4 | 4 |
| 1.5:40:28.5:26.69:3.31 | 1.6 | 0.7 | 4 |
| 1.5:40:28.5:25:5 | 1.5 | 0.5 | 4 |
| 1.5:40:28.5:22.48:7.52 | 1.7 | 0.4 | 4 |
| 1.5:40:28.5:22.43:7.57 | 2 | 0.7 | 4 |
| 1.5:40:28.5:21.53:8.47 | 1.9 | 0.6 | 4 |
| 1.5:40:28.5:20.29:9.71 | 1.8 | 0.4 | 4 |

TABLE 9
EPO Expression of cKK-E10 with Triolein

| LNP Formulation (DMG-PEG2k:cKK- | | | |
|---|---|---|---|
| E10:Chol:DOPE:Triolein) | Mean | SD | N |
| 1.5:40:28.5:30:0 (Control) | 1 | 0.28 | 4 |
| 1.5:40:28.5:28.94:1.06 | 0.85 | 0.3 | 4 |
| 1.5:40:28.5:28.93:1.07 | 0.87 | 0.25 | 4 |
| 1.5:40:28.5:28.83:1.17 | 0.78 | 0.3 | 4 |
| 1.5:40:28.5:26.69:3.31 | 0.81 | 0.22 | 4 |
| 1.5:40:28.5:25:5 | 1.22 | 0.36 | 4 |
| 1.5:40:28.5:22.48:7.52 | 1.3 | 0.47 | 4 |
| 1.5:40:28.5:22.43:7.57 | 0.87 | 0.36 | 4 |
| 1.5:40:28.5:21.53:8.47 | 1.28 | 0.56 | 4 |
| 1.5:40:28.5:20.29:9.71 | 1.24 | 0.29 | 4 |

### Example 4—Improvement in HAI Using Tricaprin

The ability of LNPs to induce immune responses in animals was determined. Groups of Balb/c mice (Mus musculus) as per the treatment group were immunized under isoflurane anesthesia with a dose of 0.4 ug per mouse in 0.05 mL of Modified Tasmania H3 mRNA-lipid nanoparticles via the IIM route in the quadriceps, on day 0 in one hind leg and day 21 in the contralateral leg. Mice were evaluated for a minimum of 3 days post-administration and any animal that lost displayed severe clinical signs after the veterinarian's assessment was euthanized by administration of 5 mg/kg of meloxicam by subcutaneous injection.

Blood was collected via submandibular or orbital sinus bleeds (in-life bleeds were performed on day −1 and on day 20) and cardiac puncture (terminal bleed, day 35) from all animals under sedation. Mice were bled on pre-study to obtain a base-line pre-immune serum sample and for pre-screening purposes.

HAI assays were performed using the A/Tasmania/503/2020 (H3N2) virus stocks (BIOQUAL, Inc.). Sera were treated with receptor-destroying enzyme (RDE) by diluting one—part serum with three parts enzyme and incubated overnight in a 37° C. water bath. Enzyme was inactivated by a 30-minute incubation period at 56° C. followed by addition of six parts PBS for a final dilution of 1/10. HAI assays were performed in V-bottom 96-well plates using four hemagglutinating units (HAU) of virus and 0.5% turkey RBC. The reference serum for each strain was included as a positive control on every assay plate. Each plate also included a back-titration to confirm the antigen dose (4 HAU/25 pl) as well as a negative control sample (PBS or naive control serum). The HAI titer was determined as the highest dilution of serum resulting in complete inhibition of hemagglutination. Results were only valid for plates with the appropriate back-titration result (verifying 4 HAU/25 ul added) and a reference serum titer within 2-fold of the expected titer.

The HAI titers were increased using tricaprin for both cKK-E10 (Table 10) and OF-02 (Table 11) at the composition of 1.5:40:28.5:25:5 (DMG-PEG-2000:Ionizable lipid:cholesterol:DOPE:glyceride).

TABLE 10
HAI Assay with cKK-E10 LNPs

| Composition | Mean | SD |
|---|---|---|
| cKK-E10 + tricaprin | 381 | 5.19 |
| cKK-E10 − tricaprin | 233 | 1.79 |

TABLE 11
HAI Assay with OF-02 LNPs

| Composition | Mean | SD |
|---|---|---|
| OF-02 + tricaprin | 830 | 2.65 |
| OF-02 − tricaprin | 427 | 1.72 |

### Example 5—Synthesis of Representative Cationic Lipids of Formula CAT-V

The cationic lipid IM-001 can be synthesized according to the general procedure set out in Scheme 1:

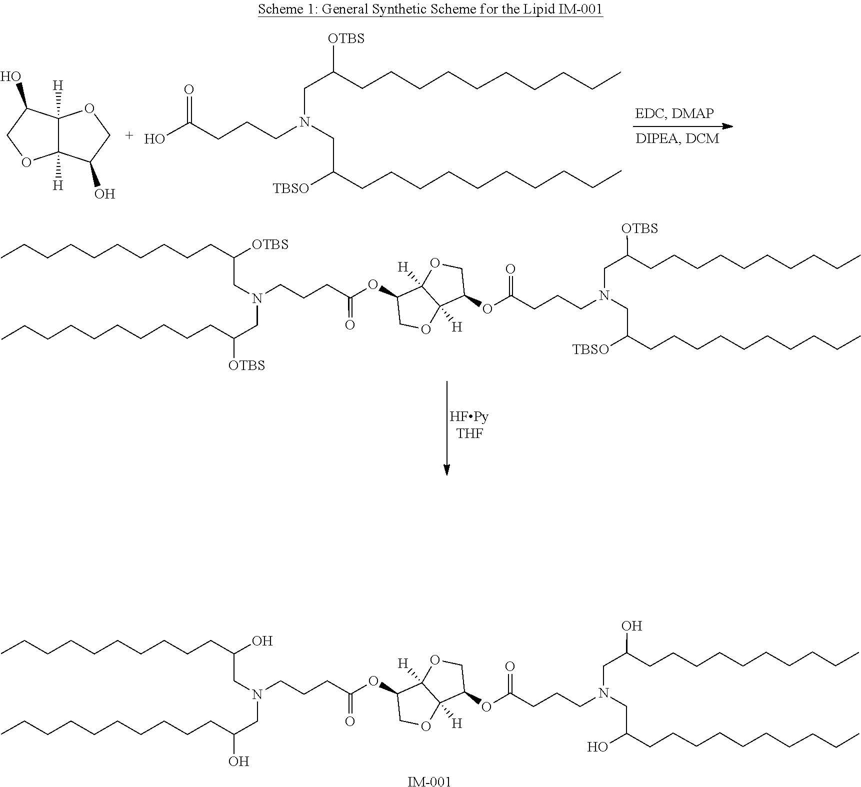

Similarly, the cationic lipid IS-001 can be synthesized according to the general procedure set out in Scheme 2:

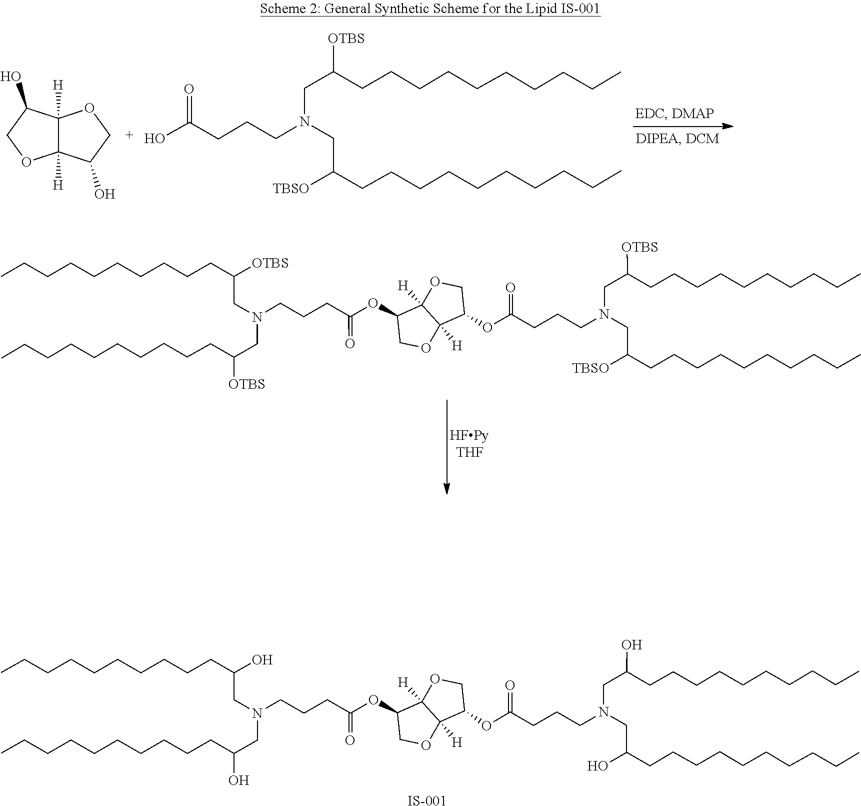

### Example 6—Synthesis of Representative Cationic Lipid of Formula CAT-VI

The cationic lipid A2H7iiT6 can be synthesized according to the procedure set out in Scheme 3:

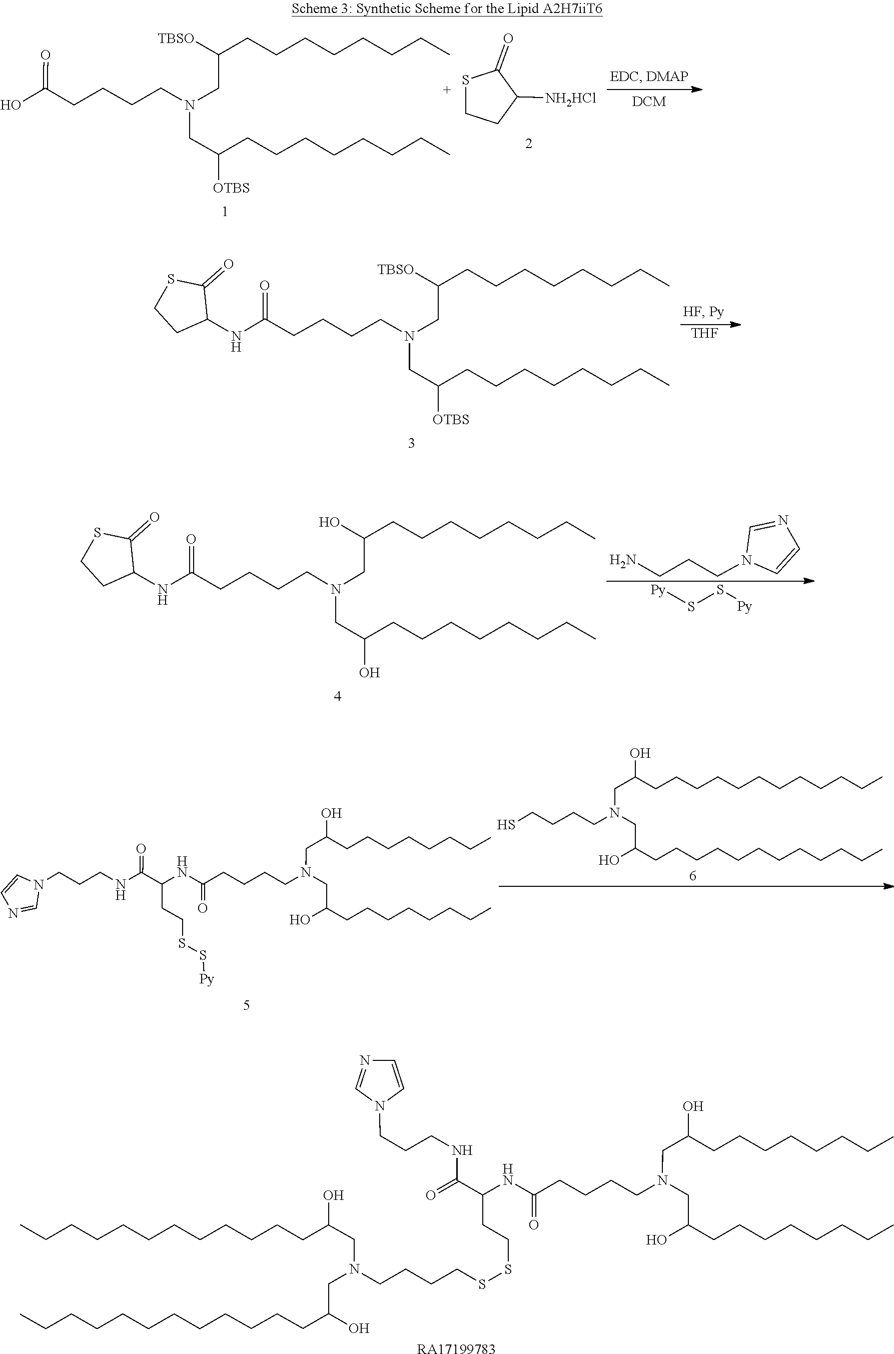

### Amide Synthesis (3)

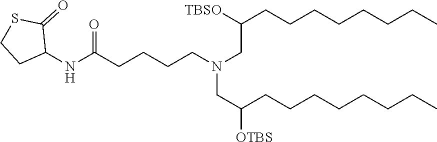 [SMILES: CCCCCCCCC(CN(CCCCC(=O)NC1CCSC1=O)CC(CCCCCCCC)O[Si](C)(C)C(C)(C)C)O[Si](C)(C)C(C)(C)C]

To a solution of acid (1) (5.14 g, 7.81 mmol), 4-Dimethylaminopyridine (DMAP) (1.60 g, 13.02 mmol), and 1-Ethyl-3-(3-dimethylaminopropyl)carbodiimide (EDC) (2.50 g, 13.02 mmol) in Dichloromethane (DCM) was added amine (2) (1.00 g, 6.51 mmol). The resulting mixture was stirred at room temperature for overnight. After 16 h, MS analysis indicated completion of the reaction. The reaction mixture was diluted with DCM and washed with saturated sodium bicarbonate (NaHCO₃) solution, water, and brine solution. The organic layer was dried over anhydrous sodium sulfate (Na₂SO₄) and concentrated. The crude residue was purified through a 80 g silica column and the desired product was eluted at 9% ethyl acetate in hexanes. The purest fractions were concentrated to obtain 3.71 g (75.3%) of pure product.

ESI-MS analysis: Calculated C₄₁H₈₄N₂O₄SSi₂, [M+H⁺]=757.58, Observed=757.5.

### Amide Deprotection (4)

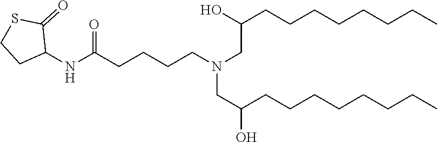 [SMILES: CCCCCCCCC(O)CN(CCCCC(=O)NC1CCSC1=O)CC(O)CCCCCCCC]

To a solution of amide 3 (3.71 g, 4.90 mmol) in tetrahydrofuran (12 mL) was added hydrogen fluoride (70% HF.py complex, 4.41 mL, 48.99 mmol) at 0° C. and stirred at the same temperature for 10 minutes. Then reaction mixture was warmed to room temperature and stirred for 16 h. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Resulting solution was then dried over anhydrous Na₂SO₄ and concentrated. The crude residue was purified, and the desired product was eluted at 6% methanol in DCM. The fractions containing pure product were concentrated to obtain 2.07 g (79.9%) of pure product.

ESI-MS analysis: Calculated C₂₉H₅₆N₂O₄S, [M+H⁺]=529.40, Observed=529.3.

### R—SS-Py Solution Synthesis (5)

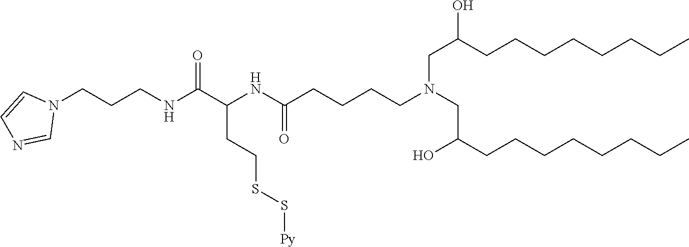

To a solution of thiolactone (4) (0.59 g, 1.116 mmol) in chloroform (20 mL) was added dipyridyl disulfide (0.74 g, 3.35 mmol) and triethylamine (TEA) (0.640 g, 3.35 mmol) and left to stir for 1 hour. Amine (5) (0.84 g, 6.70 mmol) was then added and left to stir overnight at room temperature. MS analysis indicated completion of the reaction. The reaction mixture was diluted with DCM and washed with saturated ammonium chloride solution, water, and brine solution. The organic layer was dried over anhydrous sodium sulfate (Na₂SO₄) and concentrated. The crude residue was purified through an 12 g silica column and the desired product was eluted at 7% methanol in DCM. The purest fractions were concentrated to obtain 0.72 g (84.6%) of pure product.

ESI-MS analysis: Calculated C₄₀H₇₀N₆O₄S₂, [M+H⁺]=763.50, Observed=763.5.

### Thiol Synthesis (6)

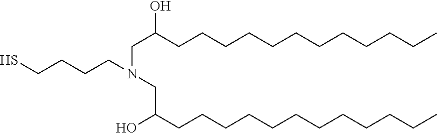 [SMILES: CCCCCCCCCCCCC(O)CN(CCCCS)CC(O)CCCCCCCCCCCC]

To a solution of 1,1′-((4-(tritylthio)butyl)azanediyl)bis(tetradecan-2-ol) (0.79 g, 1.02 mmol) in DCM (4 mL) was added trifluoracetic acid (TFA) (5.40 g, 47.38 mmol) and stirred at room temperature for 30 minutes. Triethyl silane (0.14 g, 1.18 mmol) was then slowly added and allowed to stir for an hour at room temperature. MS analysis indicated completion of the reaction. The reaction mixture was concentrated via rotary evaporator and dissolved in chloroform (12 mL) to be used soon after.

ESI-MS analysis: Calculated C₃₂H₆₇NO₂S, [M+H⁺]=530.50, Observed=530.5.

### Thiolactone Final Product Synthesis (A2H7iiT6)

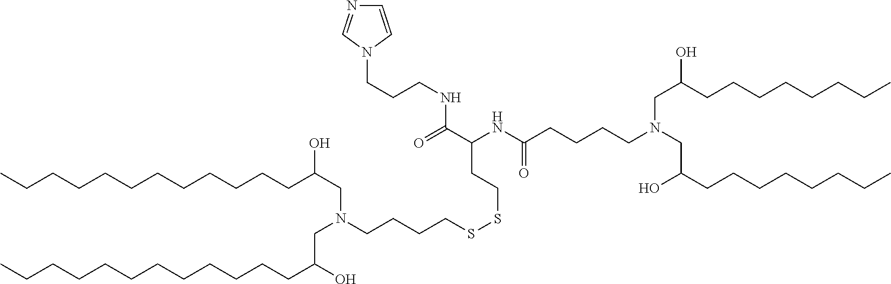 [SMILES: CCCCCCCCCCCCC(O)CN(CCCCSSCCC(NC(=O)CCCCN(CC(O)CCCCCCCC)CC(O)CCCCCCCC)C(=O)NCCCn1ccnc1)CC(O)CCCCCCCCCCCC]

To a solution of R—SS-Py (5) (0.72 g, 0.943 mmol) in chloroform (10 mL) was added a solution of thiol (6) (0.54 g, 1.02 mmol) in chloroform (8 mL) at room temperature. The resulting reaction mixture was stirred overnight for 16 h at room temperature. MS analysis indicated completion of the reaction. The reaction mixture was concentrated. The crude residue was purified through an 24 g column, and the desired product was eluted in 16% MeOH in DCM. The product containing fractions were concentrated to obtain 0.61 g (54.9%) of pure product.

ESI-MS: Calculated C₆₇H₁₃₂N₆O₆S₂, [M+H⁺]=1181.97, Observed=1181.8, [M/2+H⁺]=591.5, and [M/3+H⁺]=394.9.

### Example 7—IMEPO Data

To determine the activity of LNPs containing glycerides, EPO expression studies were conducted as described in Example 2. The data for various cationic lipid compositions, e.g., are presented in the Tables 12-14, normalized for each to the LNP formulation without addition of glycerides. Several formulations provided increased EPO expression relative to the no glyceride control condition.

TABLE 12
EPO Expression of GL-HEPES-E3-E12-DS-4-E10 LNPs

| Condition | Mean | SD | N |
|---|---|---|---|
| GL-HEPES-E3-E12-DS- | 1 | 0.3 | 4 |
| 4-E10 control / no | | | |
| glyceride | | | |
| 08:0 DG | 0.9 | 0.4 | 4 |
| 18:0-22:6 DG | 0.9 | 0.3 | 4 |
| Trioctanoin | 1 | 0.3 | 4 |
| 10:0 DG | 1 | 0.3 | 4 |
| 18:0-18:2 DG | 1.1 | 0.3 | 4 |
| 14:0 DG | 1.2 | 0.3 | 4 |
| 18:0-20:4 DG | 1.2 | 0.6 | 4 |
| 16:0 DG | 1.3 | 0.3 | 4 |
| 16:0-18:1 DG | 1.3 | 0.4 | 4 |
| 18:1 DG | 1.4 | 0.5 | 4 |
| 18:0 DG | 1.4 | 0.5 | 4 |
| 12:0 DG | 1.4 | 0.5 | 4 |
| 1-C16 Ether MG | 1.4 | 0.4 | 4 |
| 16:0-(12-PAHSA)-18:1 | 1.4 | 0.3 | 4 |
| TG | | | |
| 18:1-2:0 DG | 1.6 | 0.6 | 4 |
| 18:1 Ethylene Glycol | 1.6 | 0.7 | 4 |
| C18(plasm) MG | 1.6 | 0.4 | 4 |
| 15:0-18:1-15:0 TG | 1.6 | 0.4 | 4 |
| 16:0 Ethylene Glycol | 2 | 0.6 | 4 |

TABLE 13
EPO Expression of IM-001 LNPs

| | hEPO normalized mean | | |
|---|---|---|---|
| Condition | (6 h) | sd | n |
| IM-001 Control/ No | 1 | 0 | 4 |
| glyceride | | | |
| Diolein | 0.77 | 0.17 | 4 |
| Trilinolein | 0.79 | 0.13 | 4 |
| Tristearin | 0.85 | 0.16 | 4 |
| Triarachidin | 0.99 | 0.13 | 4 |
| Trimyristin | 1.011 | 0.3 | 4 |
| Triolein | 1.08 | 0.32 | 4 |
| Tricaprin | 1.11 | 0.22 | 4 |
| Glyceryl | 1.4 | 0.33 | 4 |
| trinonadecanoate | | | |

TABLE 14
EPO Expression of A2H7iiT6 LNPs

| | Normalized EPO | | |
|---|---|---|---|
| Condition | expression (6 h) | SD | N |
| A2H7iiT6 Control/ | 1 | 0.4 | 4 |
| No glyceride | | | |
| Tristearin | 1.9 | 0.7 | 4 |
| Glyceryl | 2.5 | 1.1 | 4 |
| Trinonadecanoate | | | |
| Trimyristin | 2.5 | 1.3 | 4 |
| Triolein | 3.1 | 1.3 | 4 |
| Diolein | 3.4 | 1.2 | 4 |
| Monoolein | 3.6 | 1.4 | 4 |
| Tricaprin | 4.1 | 1.5 | 4 |

### Example 8—IM EPO Data with POPE Helper Lipid

To determine the activity of LNPs containing glycerides, EPO expression studies were conducted as described in Example 2 but replacing DOPE helper lipid in the formulations with palmitoyloleoyl-phosphatidylethanolamine (POPE). The data for various cationic lipid compositions, e.g., are presented in Table 15, normalized for each to the LNP formulation without addition of glycerides. All conditions tested showed increased EPO expression relative to the no glyceride control.

TABLE 15
EPO Expression of GL-HEPES-E3-E12-DS-4-E10/POPE LNPs

| | hEPO normalized | | |
|---|---|---|---|
| Condition | mean | SD | N |
| GL-HEPES-E3-E12- | 1 | 0.3 | 4 |
| DS-4-E10 POPE | | | |
| control / no glyceride | | | |
| 1-C16 Ether MG | 1.2 | 0.3 | 4 |
| Trimyristin | 1.2 | 0.3 | 4 |
| 18:1-2:0 DG | 1.3 | 0.3 | 4 |
| Diolein | 1.3 | 0.4 | 4 |
| 18:1 Ethylene Glycol | 1.5 | 0.2 | 4 |
| C18(plasm) MG | 1.6 | 0.4 | 4 |
| 16:0 Ethylene Glycol | 1.6 | 0.2 | 4 |

### Example 9—Improvement in HAI Using Glycerides

The ability of glyceride LNPs to induce immune responses in animals was determined as described in Example 4. Inclusion of trimyristin and tristearin with GL-HEPES-E3-E12-DS-4-E10 LNP formulations provided increased HAI titer relative to the no glyceride control.

TABLE 16
HAI Assay with GL-HEPES-E3-E12-DS-4-E10 LNPs

| Composition | Mean | SD | N |
|---|---|---|---|
| GL-HEPES-E3-E12- | 190 | 4.76 | 8 |
| DS-4-E10 | | | |
| GL-HEPES-E3-E12- | 381 | 2.43 | 8 |
| DS-4-E10 + Trimyristin | | | |
| GL-HEPES-E3-E12- | 207 | 3.22 | 8 |
| DS-4-E10 + Tristearin | | | |

1. A composition comprising a lipid nanoparticle (LNP), wherein the LNP comprises:
(I) an ionizable lipid;
(II) a glyceride or an acylglycol; and
(III) one or more lipids selected from the group consisting of:
(a) a structural lipid;
(b) a helper lipid; and
(c) a stealth lipid.

2. The composition of claim 1, wherein the LNP comprises:
(I) an ionizable lipid;
(II) a glyceride or an acylglycol;
(III) a structural lipid;
(IV) a helper lipid; and
(V) a stealth lipid.

3. The composition of claim 2, wherein the glyceride or acylglycol is a monoglyceride, a diglyceride, a triglyceride, or a diacylglycol.

4. The composition of claim 2, wherein the glyceride or acylglycol has a structure according to Formula I or Formula II: 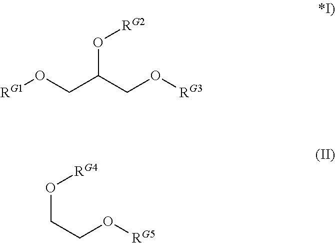
wherein:
R^{G1}, R^{G2}, and R^{G3} are each independently H, —C₍₁₋₂₅₎alkyl, —C₍₁₋₂₅₎alkenyl, —C(O)C₍₁₋₂₅₎alkyl, or —C(O)C₍₁₋₂₅₎alkenyl, wherein the —C₍₁₋₂₅₎alkyl, —C₍₁₋₂₅₎alkenyl, —C(O)C₍₁₋₂₅₎alkyl, and —C(O)C₍₁₋₂₅₎alkenyl are optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl;
provided that no more than two of R^{G1}, R^{G2} and R^{G3} are H;
R^{G4} is-C₍₁₋₂₅₎alkyl, —C₍₁₋₂₅₎alkenyl, —C(O)C₍₁₋₂₅₎alkyl, or —C(O)C₍₁₋₂₅₎alkenyl, each of which is optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl;
R^{G5} is H, —C₍₁₋₂₅₎alkyl, —C₍₁₋₂₅₎alkenyl, —C(O)C₍₁₋₂₅₎alkyl, or —C(O)C₍₁₋₂₅₎alkenyl, wherein the —C₍₁₋₂₅₎alkyl, —C₍₁₋₂₅₎alkenyl, —C(O)C₍₁₋₂₅₎alkyl, and —C(O)C₍₁₋₂₅₎alkenyl are optionally substituted with one to three groups independently selected from —OC(O)C₍₁₋₂₅₎alkyl, —OC(O)C₍₁₋₂₅₎alkenyl, —C(O)OC₍₁₋₂₅₎alkyl, —C(O)OC₍₁₋₂₅₎alkenyl, —OC₍₁₋₂₅₎alkyl, —OC₍₁₋₂₅₎alkenyl, —C(O)C₍₁₋₂₅₎alkyl, and —C(O)C₍₁₋₂₅₎alkenyl.

5. The composition of claim 2, wherein the glyceride or acylglycol is hydrolyzable by lipase.

6. The composition of claim 2, wherein the glyceride or acylglycol is selected from the group consisting of: 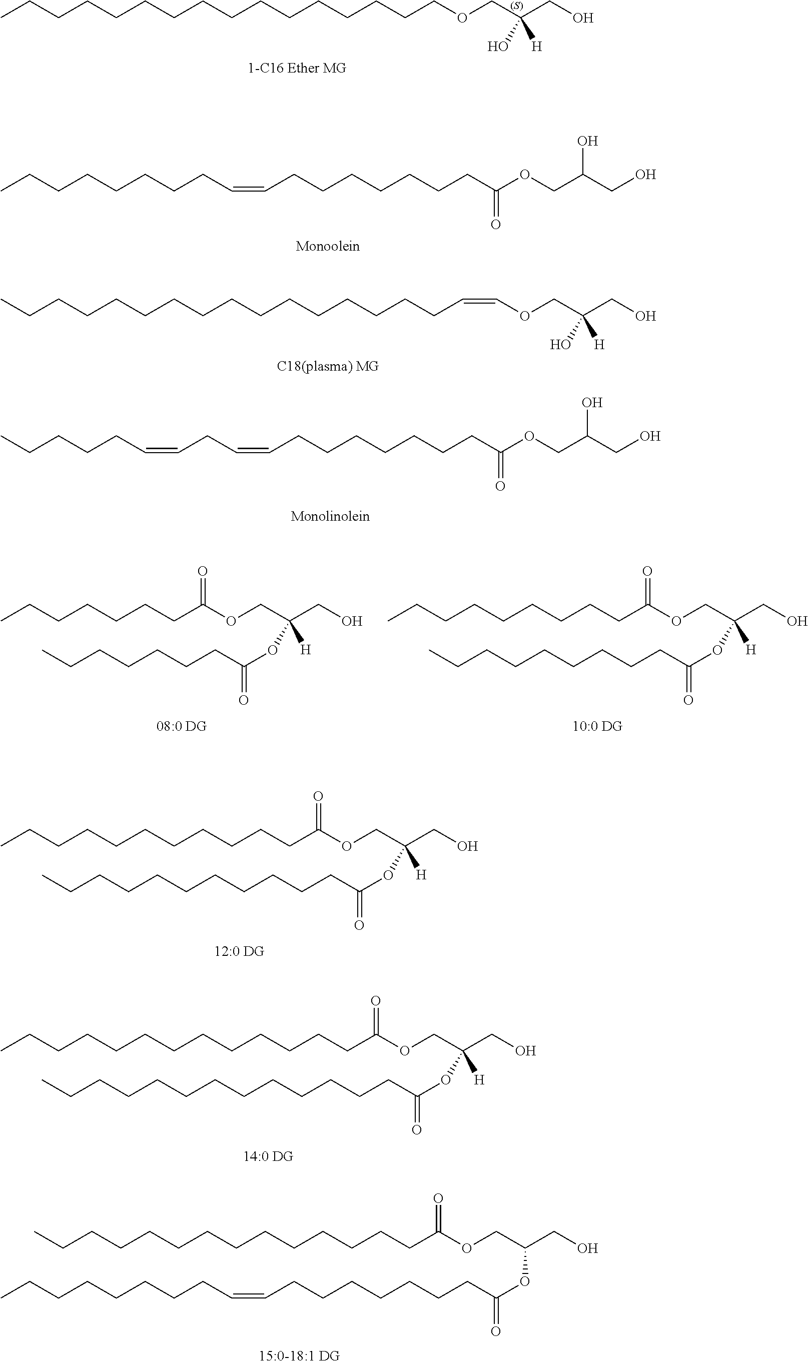 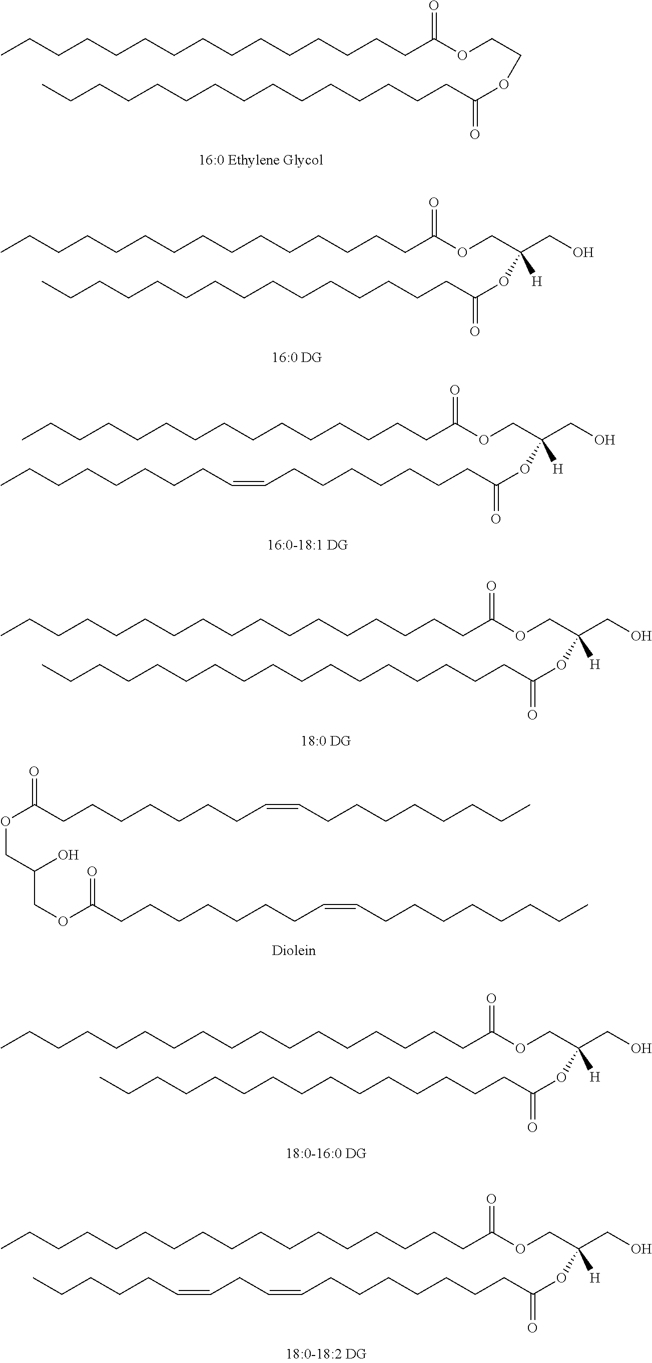 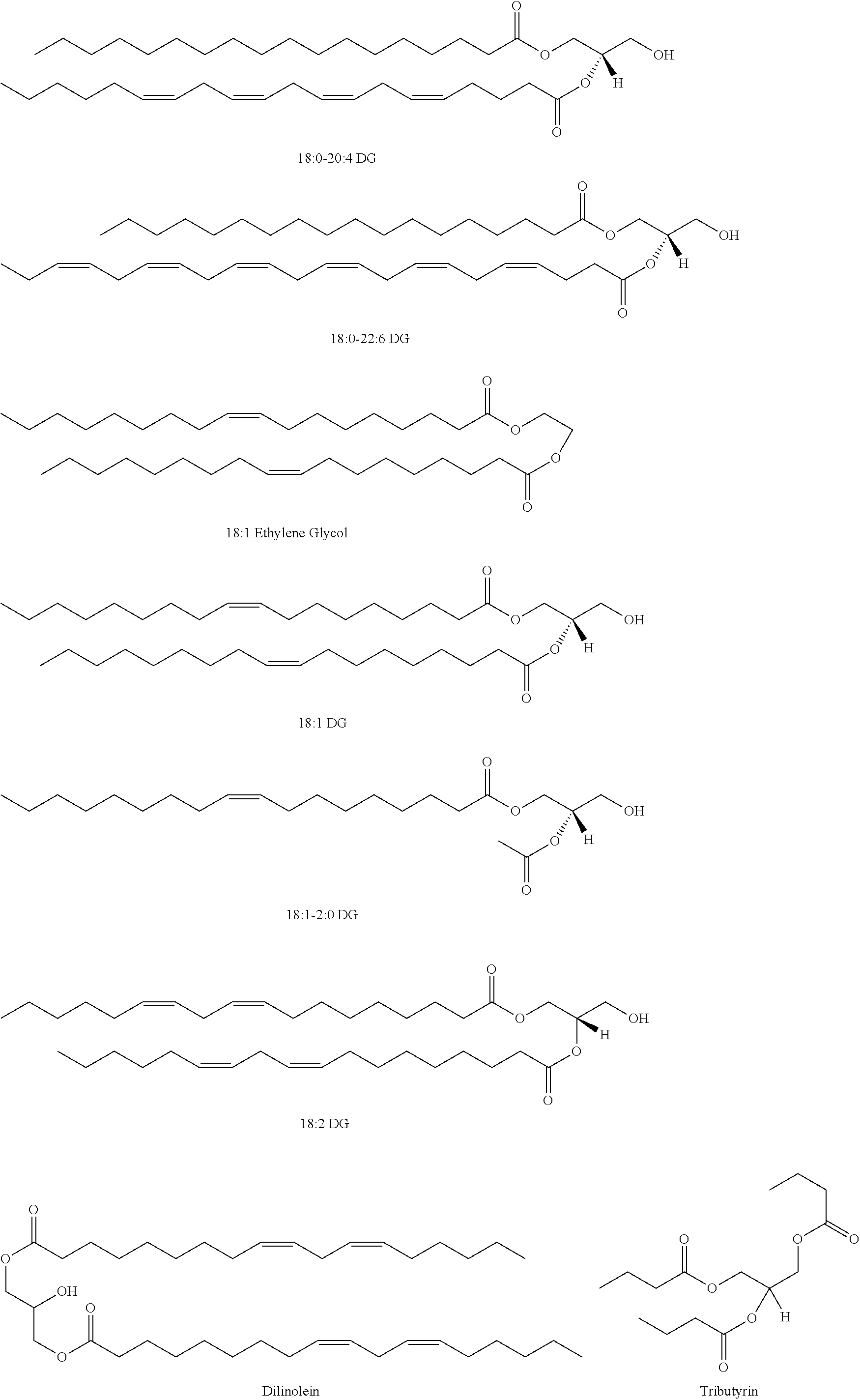 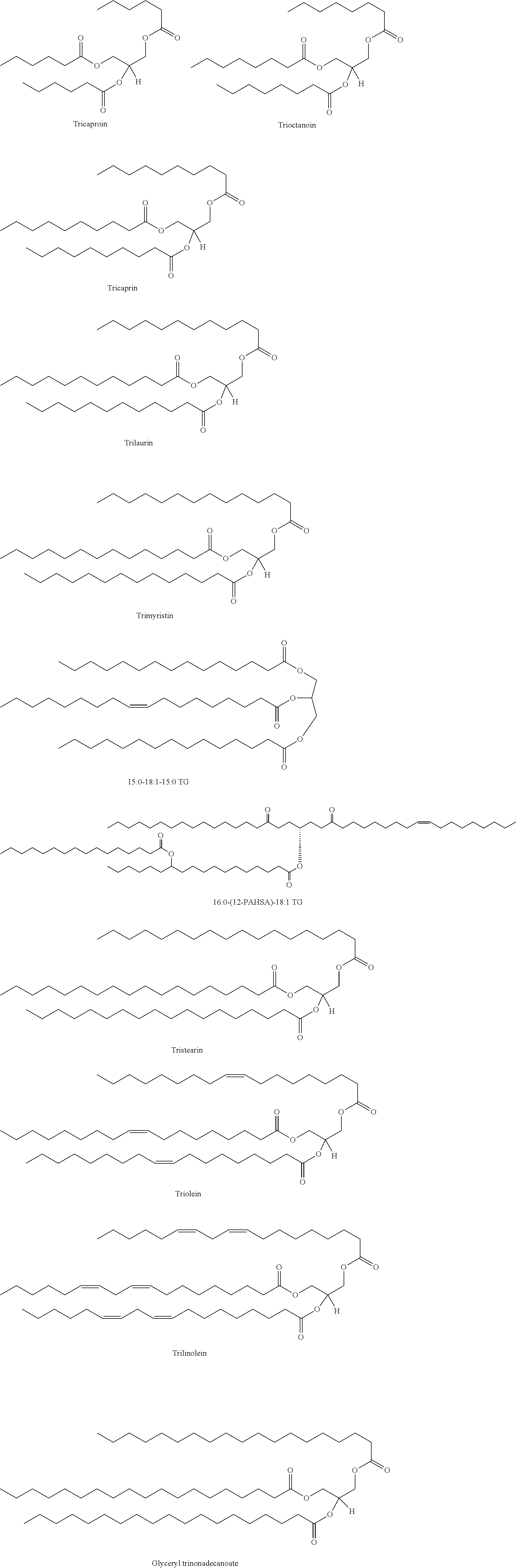 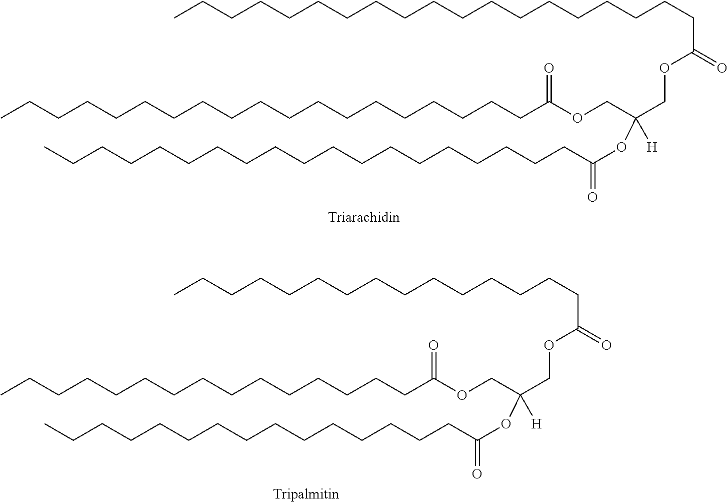 [SMILES: CCCCCCCCCCCCCCCCCCCC(=O)OCC(COC(=O)CCCCCCCCCCCCCCCCCCC)OC(=O)CCCCCCCCCCCCCCCCCCC ; CCCCCCCCCCCCCCCC(=O)OCC(COC(=O)CCCCCCCCCCCCCCC)OC(=O)CCCCCCCCCCCCCCC]
or a combination thereof.

7. The composition of claim 2, or a pharmaceutically acceptable salt thereof, wherein:
the ionizable lipid has a structure according to Formula CAT-I: 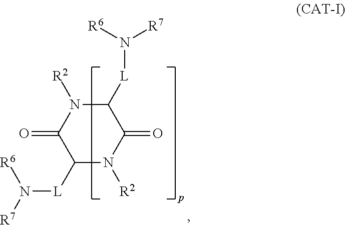
wherein:
p is an integer of between 1 and 9, inclusive;
each instance of R² is independently hydrogen or optionally substituted C₁₋₆ alkyl;
each instance of L is independently an optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, or optionally substituted heteroarylene, or combination thereof,
each instance of R⁶ and R⁷ is independently a group of formula (i), (ii), or (iii);
Formulae (i), (ii), and (iii) are: 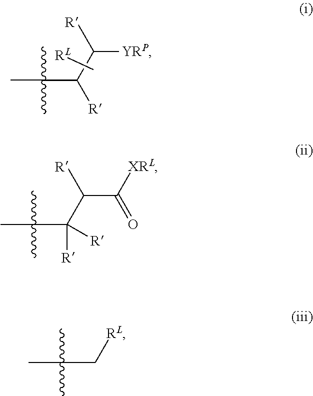
wherein:
each instance of R′ is independently hydrogen or optionally substituted alkyl;
X is O, S, or NR^{X}, wherein R^{X} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
Y is O, S, or NR^{Y}, wherein R^{Y} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
R^{P} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, or a nitrogen protecting group when attached to a nitrogen atom; and
R^{L} is optionally substituted C₁₋₅₀ alkyl, optionally substituted C₂₋₅₀alkenyl, optionally substituted C₂₋₅₀alkynyl, optionally substituted heteroC₁₋₅₀ alkyl, optionally substituted heteroC₂₋₅₀ alkenyl, optionally substituted heteroC₂₋₅₀ alkynyl, or a polymer; or
the ionizable lipid has a structure according to Formula CAT-II: 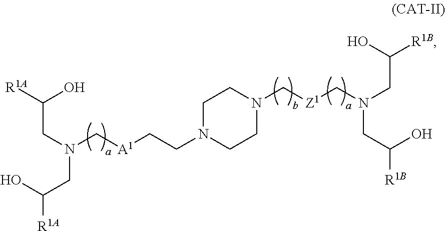
wherein:
A¹ is selected from 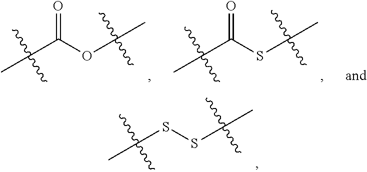
wherein the left hand side of each depicted structure is bound to the —(CH₂)a-;
Z¹ is selected from 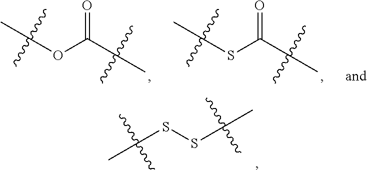
wherein the right hand side of each depicted structure is bound to the —(CH₂)a-;
R^{1A} and R^{1B} are each independently selected from optionally substituted alkyl, optionally substituted alkenyl, optionally substituted alkynyl, optionally substituted acyl, and —W¹—X¹—Y¹;
each W¹ is independently selected from optionally substituted alkyl and optionally substituted alkenyl;
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹,
each Y¹ is independently selected from hydrogen, —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to X¹;
b is 1, 2, 3, 4, or 5; and
each a is independently selected from 1, 2, 3, 4, 5, 6, 7, 8, 9, and 10.

8. The composition of claim 2, wherein the ionizable lipid is selected from the group consisting of:

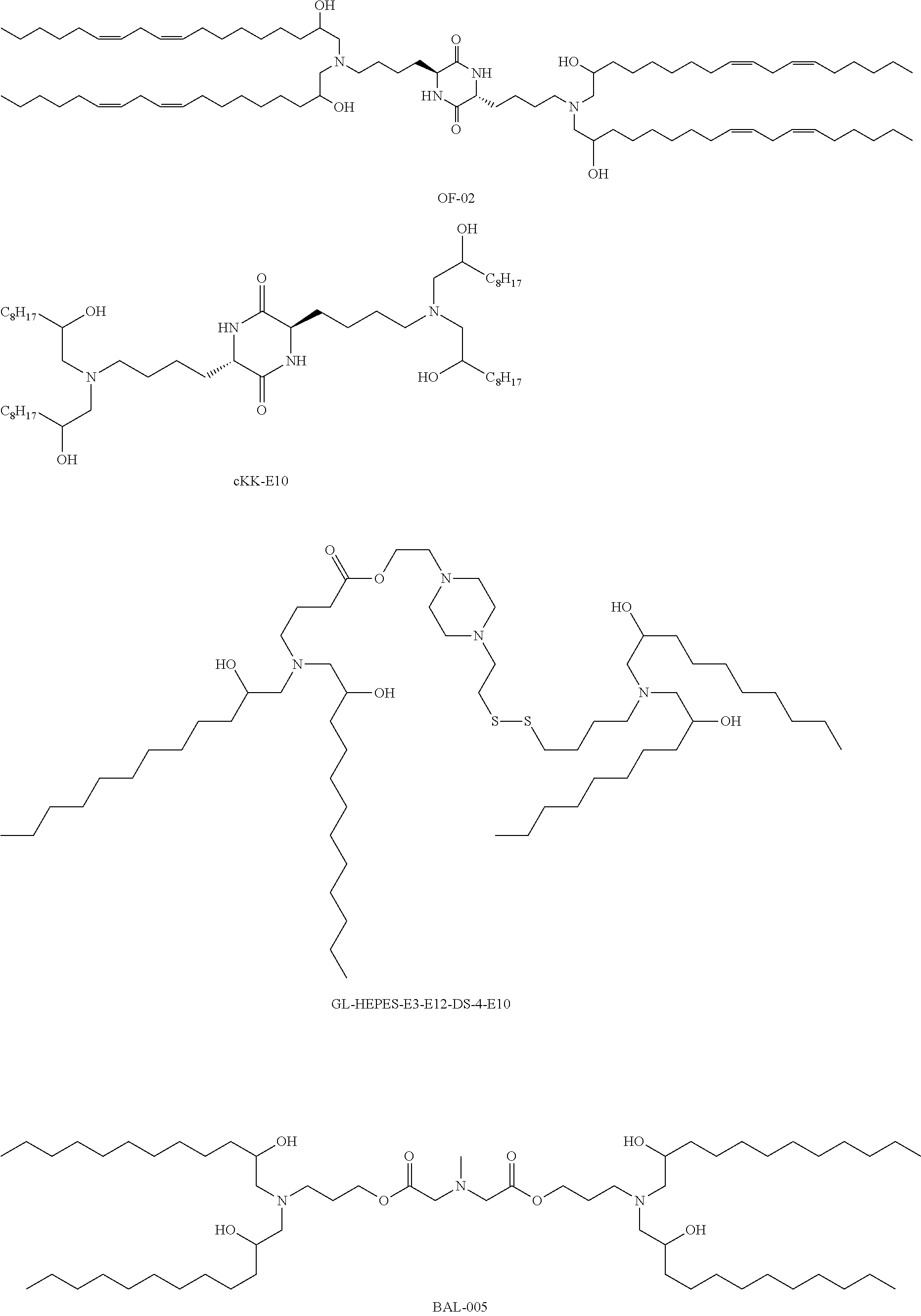

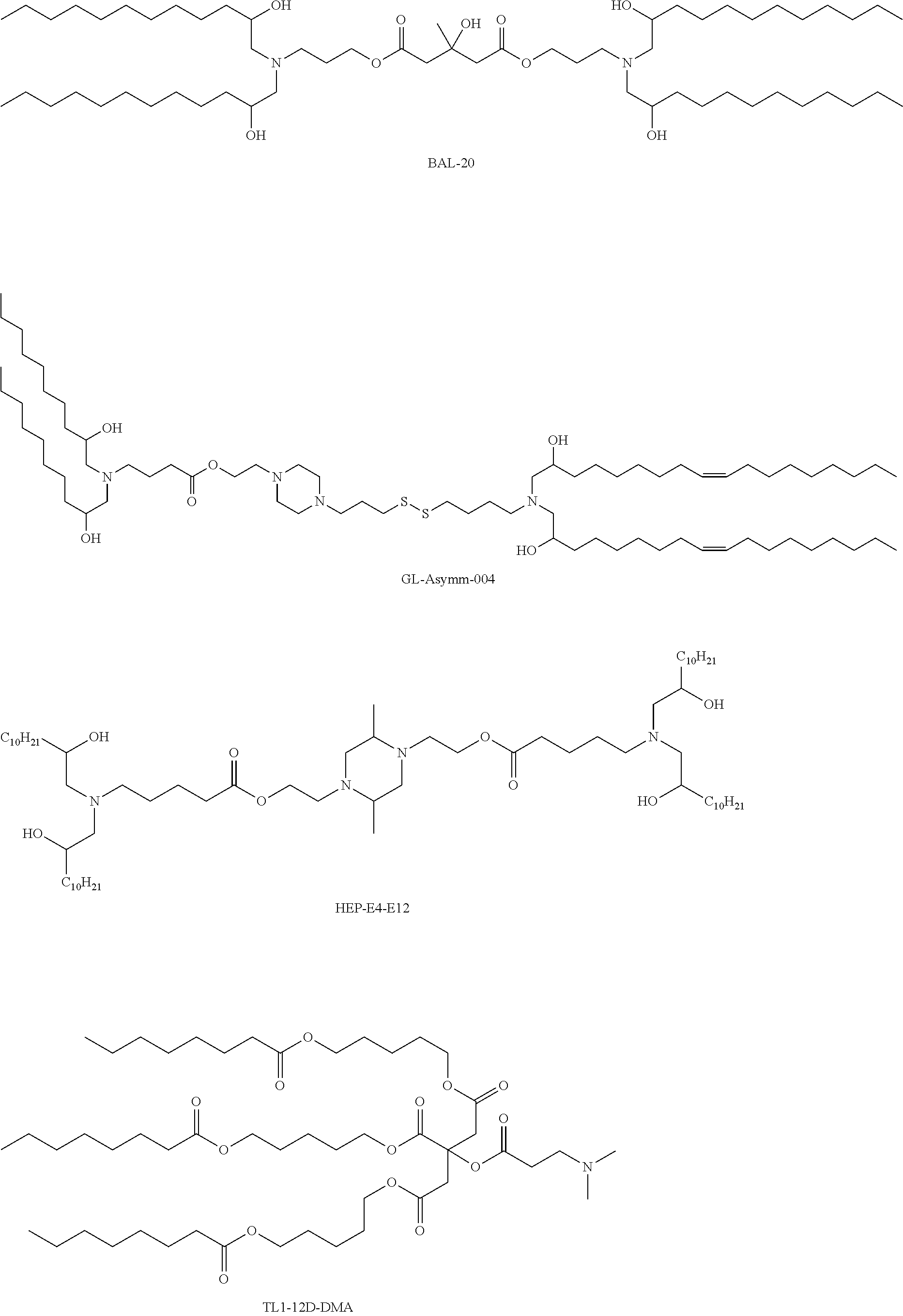

9. The composition of claim 2, wherein:
the structural lipid is a sterol, for example cholesterol;
the helper lipid is 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), 1,2-dilauroyl-sn-glycero-3-phosphocholine (DLPC), 1,2-Distearoylphosphatidylethanolamine (DSPE), or 1,2-dilauroyl-sn-glycero-3-phosphoethanolamine (DLPE); and
the stealth lipid is a polyethylene glycol-conjugated (PEGylated) lipid selected from the group consisting of 1,2-dimyristoyl-rac-glycero-3-methoxypolyethylene glycol (DMG-PEG), 1,2-distearoyl-sn-glycero-3-phosphoethanolamine-polyethylene glycol (DSPE-PEG), 1,2-dilauroyl-sn-glycero-3-phosphoethanolamine-polyethylene glycol (DLPE-PEG), and 1,2-distearoyl-rac-glycero-polyethelene glycol (DSG-PEG).

10. The composition of claim 1, wherein the LNP comprises:
the ionizable lipid at a molar ratio between 35% and 55%,
the structural lipid at a molar ratio between 20% and 35%,
the stealth lipid at a molar ratio between 0.25% and 2.75%, and
the helper lipid and the glyceride or acylglycol at a combined molar ratio of between 10% and 35%.

11. The composition of claim 1, wherein LNP comprises:
GL-HEPES-E3-E12-DS-4-E10 at a molar ratio of about 40%,
cholesterol at a molar ratio of about 28.5%,
DMG-PEG2000 at a molar ratio of about 1.5%,
DOPE at a molar ratio of about 25%,
and trimyristin at a molar ratio of about 5%.

12. The composition of claim 1, further comprising a nucleic acid molecule, wherein the nucleic acid molecule is encapsulated in the LNP.

13. The composition of claim 12, wherein the nucelic acid molecule is an mRNA molecule that encodes an antigen.

14. The composition of claim 12, wherein the composition comprises two or more LNPs, wherein each LNP encapsulates an mRNA encoding a different antigen.

15. (canceled)

16. (canceled)

17. A method of eliciting an immune response in a subject in need thereof, said method comprising administering to the subject a therapeutically effective amount of the composition of claim 12.

18. A method of preventing an infection or reducing one or more symptoms of an infection in a subject in need thereof, said method comprising administering to the subject a therapeutically effective amount of the composition of claim 12.

19. A composition comprising a lipid particle (LNP), wherein the LNP comprises:
(I) an ionizable lipid;
(II) a glyceride, wherein the glyceride is trimyristin: 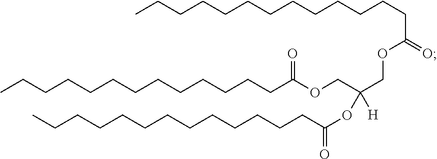 [SMILES: CCCCCCCCCCCCCC(=O)OCC(COC(=O)CCCCCCCCCCCCC)OC(=O)CCCCCCCCCCCCC]
(III) a structural lipid;
(IV) a helper lipid; and
(V) a stealth lipid.

20. The composition of claim 19, wherein the LNP comprises: (I) GL-HEPES-E3-E12-DS-4-E10; (II) trimyristin; (III) cholesterol; (IV) DOPE; and (V) DMG-PEG2000.

21. The composition of claim 19, wherein LNP comprises:
the ionizable lipid at a molar ratio of about 40%,
the structural lipid at a molar ratio of about 28.5%,
the stealth lipid at a molar ratio of about 1.5%, and
the helper lipid and the glyceride at a combined a molar ratio of about 30%.

22. A kit, wherein the kit comprises a container comprising a single-use or multi-use dosage of the composition of claim 1.


SEQUENCE LISTING (4 sequences)

SEQ ID NO: 1 - RNA, 11 nt, synthetic construct
  source 1..11: mol_type = other RNA
gggatcctacc

SEQ ID NO: 2 - RNA, 140 nt, synthetic construct
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 3 - RNA, 100 nt, synthetic construct
  source 1..100: mol_type = other RNA
cgggtggcatccctgtgacccctccccagtgcctctcctggccctggaagttgccactccagtgcccaccagccttgtcctaataaaattaagttgcatc

SEQ ID NO: 4 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa